SEQUENCE LISTING (378 sequences)

SEQ ID NO: 1 - DNA, 1203 bp, Chlamydia pneumoniae
atgagaatgctccagatttctatgcttcttttagctttaggaactgcaatcaactcaccagcaatctatgctgccgattcccaatccgtatcctttccagaacaacttccctcttcatttactggagaaattaagggaaaccacgtacggatgcgtctagcacctcatactgatgggaccatcattagggaattttctaaaggagatcttgttgctgttatcggagaaagcaaagactactacgtaatttctgcgcctccaggaattacaggttatgtgttccgctcatttgttttagataatgtcgttgaaggtgaacaagtcaatgttcgtttagaaccctcaacatcagctccagtacttgtgagactctcccgaggcacacaaatacagccagcttctcaagagccacatgggaaatggttagaggtggtcttgccctcacaatgcgtattctatgttgcaaaaaactttgttgctaacaaaggacccatcgagctgtatacgcaacgcgagggacaaaaaaagattgccatggaccttatcaattctgctttaaactttgctcatatagagcttgagaaaagcctcaatgagattgatctggaagcaatttataaaaagatcaaccttgtacaatccgaagagtttaaagatgttccaggaattcaagggcttatacaaaaagctttagaagaaatccaagatgcctatctttctaaatctctagaatctcaaaatacttcgattgcaagctcacaatgttccactcctaaggtttcttcttctgaagttacaacttcattactttcacgtcatattcgtaagcaaactgcattaaaaacagctcctcttacccaaggaagagaaaacctagagtattctctcttcagaatctgggccagtatgcagcaaggcaatgaccactctgaagcactaacacaagaagcgttttatcgcgctgaacagaagaaaaaacaagtgcttgcgggtgtattagaagtgtatcctcatgtagtaaagaacaatcccggggattacctactaaaagctcaggaaaacacgattgcttttctttacggtacaagtatcaacttagagcaatggttaggaaagcgtgtcactgtggaatgtctcccacgtcctaacaaccattttgcttttcctgcttattatgtagttggaattaaagaagcttcataa

SEQ ID NO: 2 - protein, 310 aa, Chlamydia pneumoniae
MRMLQISMLLLALGTAINSPAIYAADSQSVSFPVLLGLGQDKFLKATEDEDVLFESQKAIDAWNALLTKARDVLGLGDIGAIYQTIEFLGAYLSKVNRRAFCIASEIHFLKTAIRDLNAYYLLDFRWPLCKIEEFVDWGNDCVEIAKRKLCTFEKETKELNESLLREEHAMEKCSIQDLQRKLSDIIIELHDVSLFCFSKTPSQEEYQKDCLYQSRLRYLLLLYEYTLLCKTSTDFQEQARAKEEFIREKFSLLELEKGIKQTKELEFAIAKSKLERGCLVMRKYEAAAKHSLDSMFEEETVKSPRKDTE

SEQ ID NO: 3 - DNA, 756 bp, Chlamydia pneumoniae
ttgctaatggatatttcccatatcctggaagatcttgcctatgacgaagggatccttccaagggaagctatagaagcggctattgttaaacaaatgcaaattacgccttatttactgcatattttacacgacgctactcagcgcgtccctgagattgtaaatgatgggagttatcaaggtcacctctatgccatgtatctcctcgcacaattcagagaaagtcgcgcactccctctcatcattaaactctttgcatttgaagatgatactccacacgcaatagcaggtgatgtcctaaccgaagatctgcctaggatcctagctagcgtctgcaatgatgactcgctaattaaagagctcatagaaactccaaaaatcaatccttatgtgaaggcagccgcaatctctggtcttgtaactcttgtaggagccgggaaaattcctagggataaagttatccgttattttgcagaacttctaaactatagattagaaaaacagccctcgttcgcttgggataacctaatcgcagggatctgtactctttaccccggagagctcttctatccaataagcaaagcctttgacggaggacttgttgatacatctttcatcagcatggaagatgtcgaaaatattatccacgaagaaaccgtggaatcttgtatccataccctctgttcttctacagaactcattaatgacactctagaagaaatggaaaaatggttagaagacttccccatagaaccgtga

SEQ ID NO: 4 - protein, 251 aa, Chlamydia pneumoniae
LLMDISHILEDLAYDEGILPREAIEAAIVKQMQITPYLLHILHDATQRVPEIVNDGSYQGHLYAMYLLAQFRESRALPLIIKLFAFEDDTPHAIAGDVLTEDLPRILASVCNDDSLIKELIETPKINPYVKAAAISGLVTLVGAGKIPRDKVIRYFAELLNYRLEKQPSFAWDNLIAGICTLYPGELFYPISKAFDGGLVDTSFISMEDVENIIHEETVESCIHTLCSSTELINDTLEEMEKWLEDFPIEP

SEQ ID NO: 5 - DNA, 948 bp, Chlamydia pneumoniae
atgctagtagagttagaggctcttaaaagagagtttgcgcatttaaaagaccagaagccgacaagtgaccaagagatcacttcactttatcaatgtttggatcatcttgaattcgttttactcgggctgggccaggacaaatttttaaaggctacggaagatgaagatgtgctttttgagtctcaaaaagcaatcgatgcgtggaatgctttattgacaaaagccagagatgttttaggtcttggggacataggtgctatctatcagactatagaattcttgggtgcctatttatcaaaagtgaatcggagggctttttgtattgcttcggagatacattttctaaaaacagcaatccgagatttgaatgcatattacctgttagattttagatggcctctttgcaagatagaagagtttgtggattgggggaatgattgtgttgaaatagcaaagaggaagctatgcacttttgaaaaagaaaccaaggagctcaatgagagccttcttagagaggagcatgcgatggagaaatgctcgattcaagatctgcaaaggaaacttagcgacattattattgaattgcatgatgtttctcttttttgtttttctaagactcccagtcaagaggagtatcaaaaggattgtttgtatcaatcacgattgaggtacttattgttgctgtatgagtatacattgttatgtaagacatccacagattttcaagagcaggctagggctaaagaggagttcattagggagaaattcagccttctagagctcgaaaagggaataaaacaaactaaagagcttgagtttgcaattgctaaaagtaagttagaacggggctgtttagttatgaggaagtatgaagctgccgctaaacatagtttagattctatgttcgaagaagaaactgtgaagtcgccgcggaaagacacagaataa

SEQ ID NO: 6 - protein, 315 aa, Chlamydia pneumoniae
MLVELEALKREFAHLKDQKPTSDQEITSLYQCLDHLEFVLLGLGQDKFLKATEDEDVLFESQKAIDAWNALLTKARDVLGLGDIGAIYQTIEFLGAYLSKVNRRAFCIASEIHFLKTAIRDLNAYYLLDFRWPLCKIEEFVDWGNDCVEIAKRKLCTFEKETKELNESLLREEHAMEKCSIQDLQRKLSDIIIELHDVSLFCFSKTPSQEEYQKDCLYQSRLRYLLLLYEYTLLCKTSTDFQEQARAKEEFIREKFSLLELEKGIKQTKELEFAIAKSKLERGCLVMRKYEAAAKHSLDSMFEEETVKSPRKDTE

SEQ ID NO: 7 - DNA, 648 bp, Chlamydia pneumoniae
atgcgaaaaatgttggtattattggcatctttaggacttctatccccaaccctatccagctgcactcacttaggctcttcaggaagttatcatcctaagctatacacttcagggagcaaaactaaaggtgtgattgcgatgcttcctgtatttcatcgcccaggaaagagtcttgaacctttaccttggaacctccaaggagaatttactgaagagatcagcaaaaggttttatgcttcggaaaaggtcttcctgatcaagcacaatgcttcacctcagacagtctctcagttctatgctccgattgcgaatcgtctacccgaaacaattattgagcaatttcttcctgcagaattcattgttgctacagaactgttagaacaaaagacagggaaagaagcaggtgtcgattctgtaacagcgtctgtacgtgttcgcgtttttgatatccgtcatcataaaatagctctcatttatcaagagattatcgaatgcagccagcctttaactaccctagtcaatgattatcatcgctatggctggaactcaaaacattttgattcaacgcccatgggcttaatgcatagccgtcttttccgcgaagttgttgccagagttgagggctatgtttgtgctaactactcgtag

SEQ ID NO: 8 - protein, 173 aa, Chlamydia pneumoniae
MRKMLVLLASLGLLSPTLSSCTAITSSPGMVNLLIGWAKTKFIQPIRESKLFQSRACQITLLVLGILLVVAGLACMFIFHSQLGANAFWLIIPAAIGLIKLLVTSLCFDEACTSEKLMVFQKWAGVLEDQLDDGILNNSNKIFGHVKTEGNTSRATTPVLNDGRGTPVLSPLV

SEQ ID NO: 9 - DNA, 777 bp, Chlamydia pneumoniae
ttgggtttcactgattacttaggaggttgtttgacgaatcctttagggaaattcccctcaccacagaatccacaggttgttacgatagcgccttcttccacaacaccacaagcagtctcatctgcagttcaaggttttcttcaaactggaggagctgcctcctctacagcgacaactactaccgcatccggagcctctgcattaggactttcacctgatcaagtgcaagcgttgcttactaatttattaaatgtgggacaaccatcagtgggacaaccatcaacttcagcaggaacttcgggagcctcctcttccagtgcaagtatgcagcaacagcttttgcaacttatcttagacaagacaacaggaagtggcggatcgtccgtgagttcagagcaattacagcaactccttagcttggtgagccagatgactacgtctcaaggaggaagtggtggaactcaggcaggacaggccgcttcggtactgttgaatttgttatcggcaacaggatctgcagcagcaaatcctttagggacagctgcatcgttggcacagatcatttatgcagcagtaacaagtcctggagcaaagaaaactagcgaattttgttataattattgtggagagacctgccaaggcaactgcggttgtcctacctgtggctgtccagacggacagtgcggttgtggaggatttggccgttttttctgtggtgtatggaaaaattgttgcgggataggagagggatcccaagaacccgcaatccctttataa

SEQ ID NO: 10 - protein, 258 aa, Chlamydia pneumoniae
LGFTDYLGGCLTNPLGKFPSPQNPQVVTIAPSSTTPQAVSSAVQGFLQTGGAASSTATTTTASGASALGLSPDQVQALLTNLLNVGQPSVGQPSTSAGTSGASSSSASMQQQLLQLILDKTTGSGGSSVSSEQLQQLLSLVSQMTTSQGGSGGTQAGQAASVLLNLLSATGSAAANPLGTAASLAQIIYAAVTSPGAKKTSEFCYNYCGETCQGNCGCPTCGCPDGQCGCGGFGRFFCGVWKNCCGIGEGSQEPAIPL

SEQ ID NO: 11 - DNA, 1254 bp, Chlamydia pneumoniae
atgagccaaccccctataaaccctttaggtcaacctcaagttcctgcagcagcatccccatcagggcagccaagcgtggtaaaacgtttaaaaacgtcatccacagggttattcaaaagatttattactattcctgataaatatcctaaaatgcgctatgtctatgacacaggcattattgcccttgcggcaattgcgatcctttcgattctcctgactgcttcaggaaacagccttatgctttatgctctcgctccggcacttgccctgggagctttgggagttactctacttatttctgatattctggacagtccgaaggccaagaaaatcggtgaggcaatcactgctatcgtcgttcctatcattgtattagcgattgctgcgggtcttattgcaggggctttcgttgcctctagtgggacgatgttagtctttgccaaccctatgtttgtcatgggattgattacggtggggctatacttcatgtccttgaataagctcaccttagattatttccgtagggaacacctcttgaggatggaaaagaaaacccaagagaccgcggagcctattctagtgactccatccgccgacgatgcaaaaaaaatcgcagtggaaaagaaaaaagatctttctgcatctgcccgcatggaggaacacgaagcttcacaacgccaagatgcccgtcatcgtaggatcggtcgggaggctcaaggatctttcttctattcgtcacgaaatcctgagcatagacgctccttcggcagcctctcacgttttaaaacaaaaccctcagatgcggcttctacacgacccgcatctataagtcctccatttaaggacgattttcagccttatcacttcaaagatttaagaagcagttcattcggtagtggagcgagcagtgcgtttacacccataatgcctgcaagttcccgctctcctaatttctccacggggacggttctacaccctgagccggtctaccctaagggaggaaaagaaccctcaattcctcgagtttcttcatcttcccgccgttcccctcgtgatcgccaagataaacagcagcaacagcaaaatcaagatgaagaacagaaacagcaatctaagaagaaaagcgggaaatcgaatcaatctcttaaaactccgcctccagacggaaaaagcacggctaacctcagcccctccaatccattctctgacggttatgacgaaagagaaaaacggaaacacagaaagaacaaataa

SEQ ID NO: 12 - protein, 417 aa, Chlamydia pneumoniae
MSQPPINPLGQPQVPAAASPSGQPSVVKRLKTSSTGLFKRFITIPDKYPKMRYVYDTGIIALAAIAILSILLTASGNSLMLYALAPALALGALGVTLLISDILDSPKAKKIGEAITAIVVPIIVLAIAAGLIAGAFVASSGTMLVFANPMFVMGLITVGLYFMSLNKLTLDYFRREHLLRMEKKTQETAEPILVTPSADDAKKIAVEKKKDLSASARMEEHEASQRQDARHRRIGREAQGSFFYSSRNPEHRRSFGSLSRFKTKPSDAASTRPASISPPFKDDFQPYHFKDLRSSSFGSGASSAFTPIMPASSRSPNFSTGTVLHPEPVYPKGGKEPSIPRVSSSSRRSPRDRQDKQQQQQNQDEEQKQQSKKKSGKSNQSLKTPPPDGKSTANLSPSNPFSDGYDEREKRKHRKNK

SEQ ID NO: 13 - DNA, 657 bp, Chlamydia pneumoniae
atgttaggttctttgccatgttatcctggtgctggcaatattgaagaatacaaaaataggtatttctattgtcagttatgtgctgaggtcgttagtccctatgttgttcctgttattgtagttgatgtgcaaggggctcctcctacaggtatcttgcaggtcttgcgttgtaagcaacataaatttcaaggcctacccgtacatggccccattacttctttatgggctttggagcccgtgggtaagggagctccgcagctggagtctgcaatgtacgagctctgttctcaagtaaggaattttgacatctgctctattgtgagttgggtctttggtgggttgtgtatttttgcaggtctgattgtcggggtaatggttgaagcccctttgattgcgggattaagtgcttgggtgattccctgtatcattggaggggttggtgccattttatgcttgtttgcgatcttgatggcgtacttgggaagagggagagtccgtgagtggctcaatctttcacacgaatatataacgcaatgtcattgtcgtcagatacaggcacattctcaaaactattctgtgatcacagagtatcctgcaacctgtgcattatctcaaccgattacaaagttacctaatggatcacgcagagataactaa

SEQ ID NO: 14 - protein, 218 aa, Chlamydia pneumoniae
MLGSLPCYPGAGNIEEYKNRYFYCQLCAEVVSPYVVPVIVVDVQGAPPTGILQVLRCKQHKFQGLPVHGPITSLWALEPVGKGAPQLESAMYELCSQVRNFDICSIVSWVFGGLCIFAGLIVGVMVEAPLIAGLSAWVIPCIIGGVGAILCLFAILMAYLGRGRVREWLNLSHEYITQCHCRQIQAHSQNYSVITEYPATCALSQPITKLPNGSRRDN

SEQ ID NO: 15 - DNA, 873 bp, Chlamydia pneumoniae
atgcagattccaagaagcattggtactcacgatggttctttccatgcggatgaggtcacagcgtgtgctctccttattattttcgatcttgtggatgaaaataaaattatacgctctcgagatcctgtcgtattatcgaaatgtgaatatgtttgtgatgtcggtggtgtttattctatagaaaacaagcgttttgatcatcatcaagtctcttatgatggatcttggagtagtgcaggtatgattctgcattatcttaaagagtttggttatatggattgtgaagaatatcatttccttaacaacactttggtacatggtgtggatgaacaagataatggcagattcttctctaaggagggattttgttcgttttctgatattattaaaatttataatcctcgcgaggaagaagaaactaattcggatgcggatttttcttgtgctttgcattttaccatcgactttttgtgtcggctaaggaagaagtttcagtatgatcgagtttgtagggggattgtcagagaagccatggaaaccgaggatatgtgtttatattttgatcgtcctttagcatggcaagaaaatttcttttttttagggggagagaagcaccctgcagcttttgtttgttttccttcctgcgatcaatggattttacgagggattcctccgaatttagatcgccgtatggacgttcgtgttcctttccctgagaattgggcaggtttgttaggtaaagagttgtccaaagtatcagggattcctggggctgtgttctgccataaaggtcttttcctttctgtatggacaaatagagaaagttgccaacgtgctttgcggttaacgttacaagatcgagggatcatatga

SEQ ID NO: 16 - protein, 290 aa, Chlamydia pneumoniae
MQIPRSIGTHDGSFHADEVTACALLIIFDLVDENKIIRSRDPVVLSKCEYVCDVGGVYSIENKRFDHHQVSYDGSWSSAGMILHYLKEFGYMDCEEYHFLNNTLVHGVDEQDNGRFFSKEGFCSFSDIIKIYNPREEEETNSDADFSCALHFTIDFLCRLRKKFQYDRVCRGIVREAMETEDMCLYFDRPLAWQENFFFLGGEKHPAAFVCFPSCDQWILRGIPPNLDRRMDVRVPFPENWAGLLGKELSKVSGIPGAVFCHKGLFLSVWTNRESCQRALRLTLQDRGII

SEQ ID NO: 17 - DNA, 2058 bp, Chlamydia pneumoniae
atgtataacctactccacgcgcatcatgatgcagcctccccagacggacgactcgtttcccatttgaaaaaactctcgccccacatttacgaaggagaggtcctcattgagaatattcctgcgtactttcttggatttcatctgcctcaacagtgtatacaagtaaatttaaaaagttccttagcccaactaggtgtcgaagccgttttaaaccacttggagctaaataaagcccgaaaagaagctcgtctacacgttctcttcatgagccaagatcctatagccactgctatgttggagctcctagagcctggaagttttgtctgcaagctctttgctgctgatgatcgccgactcgtacgttcgccttgttatctcaacaggatgtttacgcacacagaccgtacaggatctccgctcctacgctttgggaaaaaacttgagcacttcatcactctagagatcattaatgatcggcttgttgtcttccttccgatccttccaggaacaatctgttacgaagagacaatttatgggttccttcccttaatgagcaaatcactcacgcgtccccatttaaaaatacgtaagtttcttcctttgtatcaaatggtaacagatcgtcctcccgttcccgaagatcataaaattcttctcataaagacagagcctctgcacatccgaaccgtatttgcaagagtcgttcaggacttactcccccaagggcttcgtcacaccgcagcggatattctcgaacctaccacacaagaatctggagatatttatgaattctacggcagcacttcagaacctattgagagaatacctttagaattttttactcttgagccttacaaagagcattcgtttttcttctatagagatatgctccaggaaaccttagaatctcctcaagaggtatttcgtgtttttgaatccataccggaaggcgaagatcaagctgcgatgtttatctccaaaggtagtgagctgcttgagctctcccaagactcttggatcatcaaacctcgaatctccccatcagatgaaagacatgctagggaaattcaaaagcacattgaagaccaaccttgtttcccttttttaaaagccatggaaacagatcatatcacaagccaaggagttttattttcccgctacttcccttcagcatcgctgaagggcatgttcctctctaactactctcgctattacctgcaacatatctattttcagattccctctcccacttctggagagtttttctcgaatcgagatcgctctttccttctcgatctatattttgcaggaatttctgtattttgggcagacttagaatcgaaacgactcttacaatacatcaaacgcagaaataaagatgtgggcatgtttgtccctaaacatcaagctgaacagtttgctcaatcctactttataggaattcatggttcctgcctaatcgctggggattatgatgagtttctccgtgagctcctgacaggaatgcatactctttctcagcaattcacgatcccagaatttccaccacagacaccgttagcaatccttacaggagggggttctggagctatggaactcgcgaatcgtgtagctacagaactctccatactctcttgtgggaatctaattagcttggataccacgaatgcctatgtagaagctaaaatgagctatgctattcctgatcttttagaacgtcaggccgacttccatgtcgaccttgctgtatttgttatcggaggcatgggaaccgatttcgaactccttctggagcttattagtctcaaaacagggaaaaaagctcttgttcccgtcttcctaatcggacctgtagactattggaaatccaagatcacagctttgtataattccaatcatgctgtaggaaccattcgaggttctgaatgggtacacaactgcctattctgcctatcctcagcaaaggcaggcattgcaatcttccgcagatatctcaatcatacgctgcccataggacctgaacaccctgtccctgaagatggttttgttatcgtttag

SEQ ID NO: 18 - protein, 685 aa, Chlamydia pneumoniae
MYNLLHAHHDAASPDGRLVSHLKKLSPHIYEGEVLIENIPAYFLGFHLPQQCIQVNLKSSLAQLGVEAVLNHLELNKARKEARLHVLFMSQDPIATAMLELLEPGSFVCKLFAADDRRLVRSPCYLNRMFTHTDRTGSPLLRFGKKLEHFITLEIINDRLVVFLPILPGTICYEETIYGFLPLMSKSLTRPHLKIRKFLPLYQMVTDRPPVPEDHKILLIKTEPLHIRTVFARVVQDLLPQGLRHTAADILEPTTQESGDIYEFYGSTSEPIERIPLEFFTLEPYKEHSFFFYRDMLQETLESPQEVFRVFESIPEGEDQAAMFISKGSELLELSQDSWIIKPRISPSDERHAREIQKHIEDQPCFPFLKAMETDHITSQGVLFSRYFPSASLKGMFLSNYSRYYLQHIYFQIPSPTSGEFFSNRDRSFLLDLYFAGISVFWADLESKRLLQYIKRRNKDVGMFVPKHQAEQFAQSYFIGIHGSCLIAGDYDEFLRELLTGMHTLSQQFTIPEFPPQTPLAILTGGGSGAMELANRVATELSILSCGNLISLDTTNAYVEAKMSYAIPDLLERQADFHVDLAVFVIGGMGTDFELLLELISLKTGKKALVPVFLIGPVDYWKSKITALYNSNHAVGTIRGSEWVHNCLFCLSSAKAGIAIFRRYLNHTLPIGPEHPVPEDGFVIV

SEQ ID NO: 19 - DNA, 273 bp, Chlamydia pneumoniae
ttggatgattcatggatcttagaggttaaagtcactccaaaagccaaagagaacaaaattgtaggctttgatggacaagctttgaaggtccgtgttaccgaacccccagaaaagggtaaggccaatgatgctgtaatttctttattagcaaaagctttatccttaccgaagcgtgatgtcactttaattgcaggagaaacttctcgaaagaaaaagtttcttcttcctaacagagttcaagacattattttttctttgcatatagacgtatag

SEQ ID NO: 20 - protein, 90 aa, Chlamydia pneumoniae
LDDSWILEVKVTPKAKENKIVGFDGQALKVRVTEPPEKGKANDAVISLLAKALSLPKRDVTLIAGETSRKKKFLLPNRVQDIIFSLHIDV

SEQ ID NO: 21 - DNA, 672 bp, Chlamydia pneumoniae
atgaccctctacttaggattgaatcaaaaaaccgctcgtaaataccaagctcattatttgcctattctaactctcttcccctatgcaaaaagcactccacaaaataagcgtgctcttcaattccttccacaagcaacccatgtgattctcacaagtccctcatccactcacctattcctttccagaatgacttctcttctttctaaggccactctaaaaacaaagacctacctctgtataggagagtccaccaaagaaagacttctctctttccttggacaagtgaagtacgtagtagcaactcaagaaatcgctgaaggcatcttcccattgctacaggcactgccctcttcagcccgcattctctacccccactcctccctcgcaagacctgtgatcagagaatttctttacaatcgatttacttttttctcttaccctcactacacagtgaagccgcgaaaacttaaaaaaaatattttatctaaatacaaaaaaattatcttcacaagcccttcaactgtaagagctttcgccaaaatctttccgcgatttcctgaaaaaacctactggtgccaaggaaggatgaccttgcaggagtttcaaaagttctcctctcaaaagcaggtatctttgttagaaacgcttgggaagtccaggacatctccgtga

SEQ ID NO: 22 - protein, 94 aa, Chlamydia pneumoniae
MTLYLGLNQKTARKYQAHYLPILTLFPYAKSLKVRVTEPPEKGKANDAVISLLAKALSLPKRDVTLIAGETSRKKKFLLPNRVQDIIFSLHIDV

SEQ ID NO: 23 - DNA, 570 bp, Chlamydia pneumoniae
atgtcatcaaatctacatcccgtaggaggaacaggaacaggagcagctgctcctgagtctgtgctaaacatagtagaggaaatagcagcatcggggagtgtcaccgctggtctacaagcaattacgtccagtccaggaatggtgaatctactcataggatgggcaaagacaaaatttattcaacctatacgtgaatcaaagctctttcaatccagagcttgccaaattaccctgctcgttttaggaattcttttggttgttgctggattagcatgtatgtttatcttccatagccagttaggggcaaatgcattttggttgattattcctgctgccataggattgattaagttactagttacatcattatgttttgatgaagcttgtacatctgaaaaactcatggttttccaaaaatgggcaggtgttttagaagatcagctcgatgatgggatccttaataactcaaataagatttttggccatgtgaaaacagaaggaaatacctctagggctactaccccagtacttaatgatggccgcggaactcctgtactttcacctttagta

SEQ ID NO: 24 - protein, 190 aa, Chlamydia pneumoniae
MSSNLHPVGGTGTGAAAPESVLNIVEEIAASGSVTAGLQAITSSPGMVNLLIGWAKTKFIQPIRESKLFQSRACQITLLVLGILLVVAGLACMFIFHSQLGANAFWLIIPAAIGLIKLLVTSLCFDEACTSEKLMVFQKWAGVLEDQLDDGILNNSNKIFGHVKTEGNTSRATTPVLNDGRGTPVLSPLV

SEQ ID NO: 25 - DNA, 828 bp, Chlamydia pneumoniae
atgcacgataaaaacaaggttctgtatctacaagcaaaccatctaaatcaaaaaagaaaacgtcataatcctctaaatacataccactcctcaaacacaactgaaactcgtcgcttaccaacatactataaatccaacattgtcttaaaaatgattttacggatctccaccgtaagccttcttacaagttgctccttctcgaaaaattctcgtacctgtttcgtcactccagaacgcattacctcacaaaaagactgccccgtccttctccatccaaaaagcactacgatttctccccctctctatgactggatctccccaaatagagaggtaatcaccgcctattctttctactgccgaggtcaaggaaactctatcataactcccgaaggggttctctatgattgtgatggactccatcacagcataactaaagaagagttccgttatatccatcctagattgattgaggtagtacgactcttgcaacaagatcaccctaaagtctctattattgaagccttttgttgtcctaaacactttcattttttagaagcctcaggaatctcactctctcaactccatctccaaggtactgcagctaccttcgctctagatcctcccctccccatggagaaactcttggcaactataaagaaactgtataaaaaaaactccgatccttctctctctaattttatcgttacagaagctacactgaccaatccagaactgcgactcacgcaacaagatctcggctcgcatacagaaattactgtagaaattctcgataatctacaaaacaaagaggctctttcctccgcataa

SEQ ID NO: 26 - protein, 142 aa, Chlamydia pneumoniae
MHDKNKVLYLQANHLNQKRKRHNPLNTYHSSNTTETRRLPTYYKSNIVLKMILRISTVSLLTSCSFSKNSRTCFVTPERLKVRVTEPPEKGKANDAVISLLAKALSLPKRDVTLIAGETSRKKKFLLPNRVQDIIFSLHIDV

SEQ ID NO: 27 - DNA, 546 bp, Chlamydia pneumoniae
atgtccttattgaaccttccctcaagccaggattctgcatctgaggactccacatcgcaatctcaaatcttcgatcccattagaaatcgggagttagtttctactcccgaagaaaaagtccgccaaaggttgctctccttcctaatgcataagctgaactaccctaagaaactcatcatcatagaaaaagaactcaaaactctttttcctctgcttatgcgtaaaggaaccctaatcccaaaacgccgcccagatattctcatcatcactccccccacatacacagacgcacagggaaacactcacaacctaggcgacccaaaacccctgctacttatcgaatgtaaggccttagccgtaaaccaaaatgcactcaaacaactccttagctataactactctatcggagccacctgcattgctatggcagggaaacactctcaagtgtcagctctcttcaatccaaaaacacaaactcttgatttttatcctggcctcccagagtattcccaactcctaaactactttatttctttaaacttatag

SEQ ID NO: 28 - protein, 106 aa, Chlamydia pneumoniae
MSLLNLPSSQDSASEDSTSQSQIFDPIRNRELVSTPEEKVRQRLKVRVTEPPEKGKANDAVISLLAKALSLPKRDVTLIAGETSRKKKFLLPNRVQDIIFSLHIDV

SEQ ID NO: 29 - DNA, 972 bp, Chlamydia pneumoniae
atgaaacaattacttttctgtgtttgcgtatttgctatgtcatgttctgcttacgcatccccacgacgacaagatccttctgttatgaaggaaacattccgaaataattatggcattattgtttccggtcaagaatgggtaaagcgtggttctgacggcaccatcaccaaagtactcaaaaatggagctaccctgcatgaagtttattctggaggcctccttcatggggaaattaccttaacgtttccccataccacagcattggacgttgttcaaatctatgatcaaggtagactcgtttctcgcaaaaccttttttgtgaacggtcttccatctcaagaagagctgttcaatgaagatggcacgtttgtcctcacacgatggccggacaacaacgacagtgataccatcacaaagccttacttcatagaaacgacatatcaagggcatgtcatagaaggaagttatacttcctttaatgggaaatactcctcatccatccacaatggagagggagttcgttctgtgttctcctccaataacatccttctttctgaagagaccttcaatgaaggtgtcatggtgaaatataccacattctatccgaatcgcgatcccgaatcgattactcattatcaaaatggacagcctcacggcttacggctaacatatctacaaggtggcatccccaatacgatagaggagtggcgttatggctttcaagacggaacgaccatcgtatttaaaaatggttgtaagacatctgagatcgcttatgttaagggagtgaaagaaggtttagaactgcgctacaatgaacaggaaattgtagctgaagaagtttcttggcgtaatgattttctgcatggagaacgtaagatctatgctggaggaatccaaaagcatgaatggtattaccgcgggagatctgtatctaaagccaaattcgagcggctaaatgctgcaggatag

SEQ ID NO: 30 - protein, 323 aa, Chlamydia pneumoniae
MKQLLFCVCVFAMSCSAYASPRRQDPSVMKETFRNNYGIIVSGQEWVKRGSDGTITKVLKNGATLHEVYSGGLLHGEITLTFPHTTALDVVQIYDQGRLVSRKTFFVNGLPSQEELFNEDGTFVLTRWPDNNDSDTITKPYFIETTYQGHVIEGSYTSFNGKYSSSIHNGEGVRSVFSSNNILLSEETFNEGVMVKYTTFYPNRDPESITHYQNGQPHGLRLTYLQGGIPNTIEEWRYGFQDGTTIVFKNGCKTSEIAYVKGVKEGLELRYNEQEIVAEEVSWRNDFLHGERKIYAGGIQKHEWYYRGRSVSKAKFERLNAAG

SEQ ID NO: 31 - DNA, 429 bp, Chlamydia pneumoniae
atgagtttagattttttcgaggagttctatcatcagtcaatactcaatacagggacgtccttccccgaaggatacttaaatattgcagaaatactctcttatcctcattgcactgatgctaacactgactttctctgtagccagtctgacaacgattttattattgcagaatctaaagataaactcacattatttaacgctgattttgctatttggctcgttcctgagcttgttcaaggacaggcagtcactcggggatatattgcggtttcccaaggagaaggaaactatgaaccagaaatggctttcgaagcctctggacaatacaatcagtcgtcgctgattctcgaagccctgcagttatatcttaaggatattaaagatactgaaaatgctctgcgttctttccgctttaataacgatcactag

SEQ ID NO: 32 - protein, 142 aa, Chlamydia pneumoniae
MSLDFFEEFYHQSILNTGTSFPEGYLNIAEILSYPHCTDANTDFLCSQSDNDFIIAESKDKLTLFNADFAIWLVPELVQGQAVTRGYIAVSQGEGNYEPEMAFEASGQYNQSSLILEALQLYLKDIKDTENALRSFRFNNDH

SEQ ID NO: 33 - DNA, 180 bp, Chlamydia pneumoniae
gtgtctattgccttaaaccgagaagaagtttgggataatccccatcacttaatgtttatcttaatgcaattccaacaattttcaggggaacaggatcgttttggaagtttcttagaagcaaccatccgtgatcgggtctcttttttagtcttacaagaaaagattgccactttaaagtag

SEQ ID NO: 34 - protein, 59 aa, Chlamydia pneumoniae
VSIALNREEVWDNPHHLMFILMQFQQFSGEQDRFGSFLEATIRDRVSFLVLQEKIATLK

SEQ ID NO: 35 - DNA, 675 bp, Chlamydia pneumoniae
atgcaaacccttgctcgtctatttggccaatctccatttgctcctttacaagctcatctggaaatggtggtctcttgtgtggaatacatgcttcctatattcactgctctccgagatggaagatatgaagaattattagaaatggcaaaacttgtttctgataaagagtatcaagcagattgtataaaaaatgatatgaggaatcatcttcctgcaggattattcatgccgatatctcgagcggggattctagaaattatttctatacaagatagcatcgcggatactgctgaagatgttgctatcttattaaccatcagacgattaaacttttatccatctatggaaacgctttttttccgatttttggaaaaaaatctagaagcttttgagttaactatgacattgctacatgaattcaaccaattgcttgaaagttcatttggggggaggaaggcagataaagcacgcttgcttgtagggcgtgtggctaaatctgaacatgaatcggatgttttgcaacgagaacttatgcaaatatttttttctgatgattttataattcctgaaaaagagttttatctttggttacaagtaattcgacgcactgcggggatttcagatagttctgaaaagctcgcacatagaattaatatgaccctagaagaaaagtaa

SEQ ID NO: 36 - protein, 224 aa, Chlamydia pneumoniae
MQTLARLFGQSPFAPLQAHLEMVVSCVEYMLPIFTALRDGRYEELLEMAKLVSDKEYQADCIKNDMRNHLPAGLFMPISRAGILEIISIQDSIADTAEDVAILLTIRRLNFYPSMETLFFRFLEKNLEAFELTMTLLHEFNQLLESSFGGRKADKARLLVGRVAKSEHESDVLQRELMQIFFSDDFIIPEKEFYLWLQVIRRTAGISDSSEKLAHRINMTLEEK

SEQ ID NO: 37 - DNA, 2538 bp, Chlamydia pneumoniae
atggcagtacgattaattgttgatgaaggccccttgtctggtgtaatttttgttctggaagatgggataagctggtctataggacgcgactctagtgctaatgacattcctattgaagatcctaaactcggtgcatcgcaagccattatcaataagactgacggaagctactacatcacaaatttagatgatacaattcctattgttgtaaatggcgtagcgatccaagaaactacacagttaaaaaatgaagatactatcttattaggaagcaatcagtattctttcttatcagatgaatttgatcctcaagatcttgtttatgattttgatattcccgaagaaaatttttctaatgattcaggggatttgtccgatagtaatgaacagggaaaagatcttgagcctcggcaaacttcggaaacaaatcattcaccgaagcctaaggaaaagctgaccaaagatcagggaagtagcgatccaattacaagtggggatcaggagcttgctgatgcttttttagcatcagcaaaagcggaaaaaaatcaaccaagagccaaagttgctaagaagggtttaaaagaatcttcaaacgagtctttgaatccaaaggaacaaaatgcaaaggattctccgaaaggagaggaaagaaccaacaaaccccagaacgccattatggaagataacggagcttcgcctaggcaagatccgcaaccaaagtcagcagaaccctctcttaaaaacacagccagggatgagactcccttgaaagaaaataaacctgtagaagagaaggctaataagaaagcaacaccggattctccagaaaaaaaagatcaacctgaggaaggttctaaaaaggaaggctctaaaatagaagcaacacctttggattcacaaaaagaatccgaggataaggaagcagaagaagcctttgtacaagaagaagaagagaaccttacggaagataataaagaagattctgacagtgccgctgatgcaaatgacgacacggcaagtgaccatactgcagaggataacaaagaaactcctaaaaaagtcgagaacgaaaagagcgcagttctatccccatttcatgttcaagacttatttcgattcgatcaaacaatttttccagcagagattgatgatattgcgaaaaaaaatatctctgtagacttgacgcagccttctcgttttttactcaaagttttagccggagctaatattggagcagagttccatttagactcaggaaaaacctatattttaggtacggatcctacaacttgtgacatagtatttaatgacttaagtgtttctcatcaacatgctaaaattactgtcggtaatgacgggggcattcttatcgaggatctcgatagtaaaaacggtgtcattgttgaaggacgaaaaattgataagacctctacattgagctcgaatcaagttgtggctttaggaacgacattatttttacttatagatcatcatgcccccgctgatactatagttgcttctctatccccagacgattacagtttgtttgggagacagcaagacgccgaagccttagaaagacaagaggcccaagaagaagaagaaaaacaaaaacgcgctacactacccgcaggatctttcattcttaccctgtttgttggaggattggctattctctttggtataggaacagcttctcttttccataccaaagaagtggttcctttagaaaatattgattatcaagaagatcttgcccaggttatcaatcagttccctacggtgcgttatacgtttaataaaacgaacagccaacttttcttaatcggacatgtcaaaaatagtacggacaaaagcgagctgctgtataaagtagacgccctttcctttgtgaaatccgtagatgataatgttattgatgatgaagctgtttggcaggagatgaacatcctgttatcaaagcgacccgagtttaaaggcatcagcatgcattccccagaacctgggaaattcatcatcacaggctatgtcaagactgaggagcaagcagcttgcctcgttgattatttaaatatacattttaattacctctcgttactagagaataaagttgttgttgaaacccagatgttaaaagcaattgcaggccatcttcttcaaggaggttttgcaaacatccatgtggcctttgtgaacggtgaagttatccttactggttacgtcaataacgatgatgcagagaagttccgtgctgtagtgcaagagctgtcggggattcctggtgtgaggttggtcaagaattttgctgtcttactcccagctgaagagggaatcatagatttaaacctacgttaccccaatcgctatcgtgttacaggctattctagatacggagaaataagtatcaatgtagttgtcaatggcagaatcctcacaagaggggacgtgattgatggtatgacagtaacaagtatacaacctaacgcgatctttttagagaaggaagggttgaaatataaaatagactacaataaataa

SEQ ID NO: 38 - protein, 845 aa, Chlamydia pneumoniae
MAVRLIVDEGPLSGVIFVLEDGISWSIGRDSSANDIPIEDPKLGASQAIINKTDGSYYITNLDDTIPIVVNGVAIQETTQLKNEDTILLGSNQYSFLSDEFDPQDLVYDFDIPEENFSNDSGDLSDSNEQGKDLEPRQTSETNHSPKPKEKLTKDQGSSDPITSGDQELADAFLASAKAEKNQPRAKVAKKGLKESSNESLNPKEQNAKDSPKGEERTNKPQNAIMEDNGASPRQDPQPKSAEPSLKNTARDETPLKENKPVEEKANKKATPDSPEKKDQPEEGSKKEGSKIEATPLDSQKESEDKEAEEAFVQEEEENLTEDNKEDSDSAADANDDTASDHTAEDNKETPKKVENEKSAVLSPFHVQDLFRFDQTIFPAEIDDIAKKNISVDLTQPSRFLLKVLAGANIGAEFHLDSGKTYILGTDPTTCDIVFNDLSVSHQHAKITVGNDGGILIEDLDSKNGVIVEGRKIDKTSTLSSNQVVALGTTLFLLIDHHAPADTIVASLSPDDYSLFGRQQDAEALERQEAQEEEEKQKRATLPAGSFILTLFVGGLAILFGIGTASLFHTKEVVPLENIDYQEDLAQVINQFPTVRYTFNKTNSQLFLIGHVKNSTDKSELLYKVDALSFVKSVDDNVIDDEAVWQEMNILLSKRPEFKGISMHSPEPGKFIITGYVKTEEQAACLVDYLNIHFNYLSLLENKVVVETQMLKAIAGHLLQGGFANIHVAFVNGEVILTGYVNNDDAEKFRAVVQELSGIPGVRLVKNFAVLLPAEEGIIDLNLRYPNRYRVTGYSRYGEISINVVVNGRILTRGDVIDGMTVTSIQPNAIFLEKEGLKYKIDYNK

SEQ ID NO: 39 - DNA, 237 bp, Chlamydia pneumoniae
atgaaagaatttttagcctatatcattaagaatctagtggaccgccctgaagaagtccgtattaaagaagttcaggggactcacacgattatttatgaactaagtgtagctaaacctgatatcgggaagatcattggcaaagaaggccgtacgatcaaagcgattcgtactcttctggtttctgtagcaagcaggaacaatgtaagggtcagtttagaaattatggaagaaaagtag

SEQ ID NO: 40 - protein, 78 aa, Chlamydia pneumoniae
MKEFLAYIIKNLVDRPEEVRIKEVQGTHTIIYELSVAKPDIGKIIGKEGRTIKAIRTLLVSVASRNNVRVSLEIMEEK

SEQ ID NO: 41 - DNA, 228 bp, Chlamydia pneumoniae
atgtttttcgctcctcttctttatgaatcgttacggaggggtcttatgcatccaacatctcatatgcaacagcaactcgctcgcttggagtttatcaacgaccagctgactacagagttagaacatgtaaatgaattattatgtagtttaggtttccctgaaggtctcactacaatcaaggcaatcgcagaggaagtcctctctgatgacgaacctctactagattag

SEQ ID NO: 42 - protein, 75 aa, Chlamydia pneumoniae
MFFAPLLYESLRRGLMHPTSHMQQQLARLEFINDQLTTELEHVNELLCSLGFPEGLTTIKAIAEEVLSDDEPLLD

SEQ ID NO: 43 - DNA, 1269 bp, Chlamydia pneumoniae
atgaaaaaacaggtatatcaatggttagcgagtgtggttcttttagcgctgacaatttcaggatacgctgaacttcctctctcggaacaaaaagtaaaaagtcacacttatacaactttagacgaagtcaaagactacttaagtaaacggggttttgtagaaacgcgaaagcaagatggcgttttaagaatagcaggagatgttagagcccggtggttgtatttcagagaagatatcaaaaacccctcagataaagataaatacaatcccttaccagtaaatcgttatcgtagtgaattttatctctatattgattatcgcgctgagaggaactggctgtcttcaaagatgaattggacagcaattgcaggaggggaaaacactgcagctggtgttgatatcaacagagcatttctaggatatcgtttttataagaatcccgaaacacgtacagatttctttatggaaatcggacgttctggtttaggagatctctttgagtcagaagtccaattccaaagtaattttgacggactacatatatattggactcgagaactttctaaggactatccttatcaagtgattgttcatggaggtcctttcgtcgtgaacatgacaaaaaaacattatgcttgggttgtagaagggattctcaatcgtttgcctaaacagttttttgtgaaatgtagtgttgtcgactggaacacattcgttccttcagaaacctccactacagaaaaagctgctacaaacgctatgaaatacaaatactgtgtttggcagtggctcgtcggaaagcatagtcaggttccttggatcaatggacagaaaaagcctctatatctttatggagctttcttaatgaaccctttagcaaaggctacgaagactacgttaaatggaaaagaaaacctagcttggtttattggaggaactttagggggactcagaaaagctggagactggtctgccacagtacgttatgagtatgtcgaagccttgtcggttccagaaatagatgtttcagggattggccgtggtaatttattaaagttttggttcgcccaagcaattgctgctaactatgatcctaaagaggctaatggttttacaaattataaaggattttccgctctatatatgtatggcatcacagattctctatcattcagagcttatggggcttactccaaaccagcaaacgataaactcggcagtgattttactttccgaaagtttgatctaggtataatttcagcgttttaa

SEQ ID NO: 44 - protein, 422 aa, Chlamydia pneumoniae
MKKQVYQWLASVVLLALTISGYAELPLSEQKVKSHTYTTLDEVKDYLSKRGFVETRKQDGVLRIAGDVRARWLYFREDIKNPSDKDKYNPLPVNRYRSEFYLYIDYRAERNWLSSKMNWTAIAGGENTAAGVDINRAFLGYRFYKNPETRTDFFMEIGRSGLGDLFESEVQFQSNFDGLHIYWTRELSKDYPYQVIVHGGPFVVNMTKKHYAWVVEGILNRLPKQFFVKCSVVDWNTFVPSETSTTEKAATNAMKYKYCVWQWLVGKHSQVPWINGQKKPLYLYGAFLMNPLAKATKTTLNGKENLAWFIGGTLGGLRKAGDWSATVRYEYVEALSVPEIDVSGIGRGNLLKFWFAQAIAANYDPKEANGFTNYKGFSALYMYGITDSLSFRAYGAYSKPANDKLGSDFTFRKFDLGIISAF

SEQ ID NO: 45 - DNA, 1596 bp, Chlamydia pneumoniae
atgttgggcaaagaagaagagtttacgtgtaaacaaaagcagtgtttgtcacattttgttaccaatctgacgtccgatgtatttgctttaaaaaatcttccagaagtcgttaagggagctttattttctaaatactcccgttcagttttaggtttgcgagcacttttgttaaaagaatttctatctaatgaagaggatggagatgtttgtgacgaagcctatgacttcgaaaccgatgtacagaaagctgcggacttttaccaaagggttcttgataattttggggatgattctgtaggagagcttggcggagcccacctggctatggaaaatgtctctattttggctgctaaagttttagaggatgctcgaattggcggatccccgctagaaaagtccacaagatacgtctatttcgatcaaaaggtacggggggagtatttatattaccgagaccctattttgatgacttcggcctttaaagacatgtttttgggtacttgtgattttttattcgatacctattctgctttaatccctcaagttcgtgcctattttgaaaaactgtatcctaaagattctaaaacacccgcatctgcctatgccacatcattacgagctaaagttttagattgtatacggggacttcttcctgcggcaactttgacaaatctaggatttttcggtaacggtaggttttggcaaaatctgattcacaagttacaaggtcataaccttgcagagttgcgacgtttaggagatgaatccctaacagagcttatgaaagttattccttcatttgtaagtagagccgagcctcatcatcaccatcatcaagctatgatgcaatatcgaagagctttaaaagagcagctcaagggacttgctgaacaagcaacatttagtgaggagatgtcttcttcaccgagtgttcagttggtatacggagaccctgatggcatttataaagtagctgctggatttctttttccttattcaaatcgttctcttacagatctcatagactattgtaaaaaaatgcctcatgaagatcttgtacagattttagagagcagtgtttctgcaagagaaaaccgccggcataagtctcctcgtggtttagaatgcgtagaatttggctttgatatacttgctgatttcggtgcataccgcgatttgcaacgacatcggacgctgactcaagaacgacagttactctctacacatcatggatacaattttcctgtggagcttctagatactcctatggaaaaatcttatcgagaagctatggagagggcgaatgaaacctataatgagattgttcaggagttccctgaggaagctcagtatatggttcccatggcttacaatatacgttggtttttccatgtaaatgctcgggctttgcaatggatttgtgagttacgctcacagcctcaaggtcatcaaaattaccgcactatagctacaggtttagtgcgagaggttgtcaagttcaatcctatgtacgaattatttttcaaatttgtagattattctgacatagatttaggacggttaaatcaggaaatgcgaaaagaaccaacgacctaa

SEQ ID NO: 46 - protein, 531 aa, Chlamydia pneumoniae
MLGKEEEFTCKQKQCLSHFVTNLTSDVFALKNLPEVVKGALFSKYSRSVLGLRALLLKEFLSNEEDGDVCDEAYDFETDVQKAADFYQRVLDNFGDDSVGELGGAHLAMENVSILAAKVLEDARIGGSPLEKSTRYVYFDQKVRGEYLYYRDPILMTSAFKDMFLGTCDFLFDTYSALIPQVRAYFEKLYPKDSKTPASAYATSLRAKVLDCIRGLLPAATLTNLGFFGNGRFWQNLIHKLQGHNLAELRRLGDESLTELMKVIPSFVSRAEPHHHHHQAMMQYRRALKEQLKGLAEQATFSEEMSSSPSVQLVYGDPDGIYKVAAGFLFPYSNRSLTDLIDYCKKMPHEDLVQILESSVSARENRRHKSPRGLECVEFGFDILADFGAYRDLQRHRTLTQERQLLSTHHGYNFPVELLDTPMEKSYREAMERANETYNEIVQEFPEEAQYMVPMAYNIRWFFHVNARALQWICELRSQPQGHQNYRTIATGLVREVVKFNPMYELFFKFVDYSDIDLGRLNQEMRKEPTT

SEQ ID NO: 47 - DNA, 1206 bp, Chlamydia pneumoniae
atgttattggtaaggaaatggttgcatacttgtttcaaatattggatttactttcttccggtggtaacgctacttcttcccctagtgtgttacccttttctgtcgattagtcaaaaaatttatggatactttgtttttactacaatttcttctttaggctggttttttgcattgagacgtagggaaaatcaattaaaaacagcagctgttcagcttcttcaaacaaaaattagaaaattaacagaaaataatgaagggttaagacaaattcgagaatctcttaaagaacatcagcaagagagtgctcaactgcaaattcaaagtcagaagcttaaaaacagcctatttcatcttcagggtttacttgtgaaaactaagggagaggggcaaaaattagaaactttgttacttcatagaacagaagagaatcgatgtttgaaaatgcaagtagattctttaattcaggaatgcggagaaaaaacagaggaagtacaaactttaaatcgagagttggctgagactttagcctaccagcaagctttaaatgacgagtatcaagcgaccttctctgagcaacgcaatatgctggataagcggcagatctacattggaaagctggaaaacaaggttcaggatttaatgtatgagatccgtaacttgcttcagttagagtcagacatagcagagaatattccttctcaagaatcgaatgctgttacgggaaatatttctttacaattgtctagtgagttaaaaaaaattgcttttaaggctgaaaacatagaggcagcctcttctttaacagcatcacgttaccttcatacagatacgagtgtgcataactactctttagagtgtcgccagttatttgatagcttaagagaagaaaatctcgggatgctttttgtctatgctcgtcaatcccaacgtgcggtttttgctaatgcgttatttaaaacgtggacggggtattgtgcagaagattttttaaaatttggtagtgacatagtgatttctgggggcaaacagtggatggaggatcttcattcctctagagaagaatgctctggtagattagtgattaaaacgaaatcacgaggtcatcttcctttccgttattgtttaatggctttgaataaaggccctctttgctatcatgttttgggggttctttatcctctccataaagaagtgcttcagagttga

SEQ ID NO: 48 - protein, 401 aa, Chlamydia pneumoniae
MLLVRKWLHTCFKYWIYFLPVVTLLLPLVCYPFLSISQKIYGYFVFTTISSLGWFFALRRRENQLKTAAVQLLQTKIRKLTENNEGLRQIRESLKEHQQESAQLQIQSQKLKNSLFHLQGLLVKTKGEGQKLETLLLHRTEENRCLKMQVDSLIQECGEKTEEVQTLNRELAETLAYQQALNDEYQATFSEQRNMLDKRQIYIGKLENKVQDLMYEIRNLLQLESDIAENIPSQESNAVTGNISLQLSSELKKIAFKAENIEAASSLTASRYLHTDTSVHNYSLECRQLFDSLREENLGMLFVYARQSQRAVFANALFKTWTGYCAEDFLKFGSDIVISGGKQWMEDLHSSREECSGRLVIKTKSRGHLPFRYCLMALNKGPLCYHVLGVLYPLHKEVLQS

SEQ ID NO: 49 - DNA, 675 bp, Chlamydia pneumoniae
atgacatcctggatagaattacttgataagcaaattgaagatcaacatatgttaaagcacgaattttatcagcgttggtctgaaggaaagttagaaaaacaacaacttcaagcttatgccaaagattactatttacatattaaagcatttccttgttacctttcagcgctgcatgctcgctgtgatgacttgcagattcgtagacaaattcttgagaatctcatggatgaagaagctggaaatcctaatcacatagatttatggagacagtttgctttatctcttggagtttctgaagaggagcttgccaatcatgaattcagtcaggctgctcaagatatggtagcgacatttcgccgcttatgcgacatgccacaacttgccgtgggtttaggcgctctctatacttatgagattcagattcctcaagtctgtgtagagaaaatccgtggtttgaaagaatattttggagtttctgctcgaggctatgcatactttactgtacatcaagaagctgatattaaacatgccagcgaagagaaagaaatgctacaaactttggtaggcagagagaatcctgatgctgttttgcaaggatcacaagaagttttagatactctatggaactttttgagctcttttattaattcaacggagccttgttcttgtaagtag

SEQ ID NO: 50 - protein, 224 aa, Chlamydia pneumoniae
MTSWIELLDKQIEDQHMLKHEFYQRWSEGKLEKQQLQAYAKDYYLHIKAFPCYLSALHARCDDLQIRRQILENLMDEEAGNPNHIDLWRQFALSLGVSEEELANHEFSQAAQDMVATFRRLCDMPQLAVGLGALYTYEIQIPQVCVEKIRGLKEYFGVSARGYAYFTVHQEADIKHASEEKEMLQTLVGRENPDAVLQGSQEVLDTLWNFLSSFINSTEPCSCK

SEQ ID NO: 51 - DNA, 1287 bp, Chlamydia pneumoniae
atggatataaaaaaactcttttgcttatttctatgttcttctctaattgccatgagtcccatttatgggaaaacaggtgactatgagaaactcacccttacagggatcaatatcattgatagaaacggcctgtcagaaactatttgctctaaagagaagctaaagaaatacaccaaggtagactttcttgctccccagccctatcaaaaggtcatgaggatgtataaaaacaaacgcggagataacgtttcttgtttaacagcctatcacactaacgggcaaattaagcagtacctggagtgtctcaataatcgtgcttatggaagatatcgtgaatggcacgtcaacgggaatatcaaaatccaagctgaggttatcggaggtattgcggatcttcatccctcagcagagtctggctggctatttgatcaaactacatttgcctataatgatgaaggtatcttagaagccgctatcgtctatgaaaaagggctgctcgaaggatcttcggtgtattaccatactaatgggaatatttggaaagagtgtccctatcataagggagttcctcaaggtaaattcctgacatacacatcttcggggaaactgctcaaagaacagaattaccaacaaggcaaaagacacggtctttcgattcgctacagcgaagattccgaagaagatgttttagcctgggaagaatatcatgagggacgactcctaaaagcagagtacttagatcctcaaactcacgaaatctatgcgactatacacgaagggaacggcattcaagcaatctacggcaagtatgccgttatagaaactagggcattttaccgaggggaaccttatggaaaagttaccagattcgacaactccggaacacagattgtccaaacgtataaccttttgcaaggcgcgaagcacggagaagaatttttcttttatcctgagacagggaaacccaagctgcttcttaattggcatgaaggaattttaaatgggatagtaaaaacttggtatcccggaggaaccttagaaagttgtaaagaactcgtaaataacaaaaaatccgggttactgaccatttactaccctgaaggacagatcatggcgaccgaagagtatgataatgatcttctaattaaaggagagtacttccgccctggagaccgtcatccctactctaaaatagatcgtggttgtgggactgcagtatttttctcgtcggcgggaactattactaaaaaaatcccctatcaggacggcaaacctttgctcaactag

SEQ ID NO: 52 - protein, 428 aa, Chlamydia pneumoniae
MDIKKLFCLFLCSSLIAMSPIYGKTGDYEKLTLTGINIIDRNGLSETICSKEKLKKYTKVDFLAPQPYQKVMRMYKNKRGDNVSCLTAYHTNGQIKQYLECLNNRAYGRYREWHVNGNIKIQAEVIGGIADLHPSAESGWLFDQTTFAYNDEGILEAAIVYEKGLLEGSSVYYHTNGNIWKECPYHKGVPQGKFLTYTSSGKLLKEQNYQQGKRHGLSIRYSEDSEEDVLAWEEYHEGRLLKAEYLDPQTHEIYATIHEGNGIQAIYGKYAVIETRAFYRGEPYGKVTRFDNSGTQIVQTYNLLQGAKHGEEFFFYPETGKPKLLLNWHEGILNGIVKTWYPGGTLESCKELVNNKKSGLLTIYYPEGQIMATEEYDNDLLIKGEYFRPGDRHPYSKIDRGCGTAVFFSSAGTITKKIPYQDGKPLLN

SEQ ID NO: 53 - DNA, 795 bp, Chlamydia pneumoniae
gtggaaaaacttgagtttgtcaccagcctttcttctcctgatgatgatttgattactttcaataaacagggattgattgcaggcccagaagaagaaaaggtagcgtttcttgtacgtagcaatgctatgctagatgcaggacccgaaacccccgcgtcgtttcctgaatctttaagggaacaattcgatattttccctgagtatgttgaagtgctctactctaatgaaggattagatgtctgggaagcaggatgtacgtggattctaaataatgaagtgaccatccaactgcgtaaacatcaccggaaagcttcgcgatggctaggaatgtattccagagatgaggtactcgctcacgaagccgtgcatgctgtgagaatgaaatttcatgagcctgtctttgaagaggtgttagcttatcaaacttctcgttggggttggagaaggtttttcggtcctctatttcgctctccaggagagagctacttgctattattcttcaccattttaggtttaggaatctccttatggtatcctgccggtatactgattatgctggttttacctatgtattttttgatgcgattgtgcatggcgcagagctatttgtatcgggccatgaaaaagattcgtaaaatgctcggagtacctcccttatgggtgctgctaaggctgacggataaggaaataaaaatgtttgctaaagagcctattcctgttttggaacactatgctagaaaacgaaagcttgaaaatgtccgttggaagcaaatttatcaatcctactttgtttaa

SEQ ID NO: 54 - protein, 264 aa, Chlamydia pneumoniae
VEKLEFVTSLSSPDDDLITFNKQGLIAGPEEEKVAFLVRSNAMLDAGPETPASFPESLREQFDIFPEYVEVLYSNEGLDVWEAGCTWILNNEVTIQLRKHHRKASRWLGMYSRDEVLAHEAVHAVRMKFHEPVFEEVLAYQTSRWGWRRFFGPLFRSPGESYLLLFFTILGLGISLWYPAGILIMLVLPMYFLMRLCMAQSYLYRAMKKIRKMLGVPPLWVLLRLTDKEIKMFAKEPIPVLEHYARKRKLENVRWKQIYQSYFV

SEQ ID NO: 55 - DNA, 234 bp, Chlamydia pneumoniae
atgaatgaaggtatccactctgtctgttttcaaaaaacacctcggcttactgcgaagtccgtagtgagtatggagatgctcttaactactcaacagcttccttccgcagaagggatgccctcggttgctaatttggaagcggattttttacgagcagaagctctgttagcagaaatgcgagaaattcgtggttgcttggagcaatctttgcgaacactagtccctagtgagtag

SEQ ID NO: 56 - protein, 77 aa, Chlamydia pneumoniae
MNEGIHSVCFQKTPRLTAKSVVSMEMLLTTQQLPSAEGMPSVANLEADFLRAEALLAEMREIRGCLEQSLRTLVPSE

SEQ ID NO: 57 - DNA, 1815 bp, Chlamydia pneumoniae
atgaaacatacctttaccaagcgtgttctattttttttctttttagtgattcccattcccctactcctcaatcttatggtcgtaggttttttctcattttctgccgctaaagcaaatttagtacaggtcctccatacccgtgctacgaacttaagtatagaattcgaaaaaaaactgacgatacacaagcttttcctcgatagacttgccaacacattagccttaaaatcctatgcatctccttctgcagagccctatgcacaggcatacaatgagatgatggcactctccaatacagacttttccttatgccttatagatccctttgatggatctgtaaggacgaaaaatcctggagaccctttcattcgctatctaaaacagcatcctgaaatgaagaaaaagctatccgcagctgtagggaaagcctttttattgaccattccaggtaaaccacttttacattatcttattctagttgaagatgtcgcatcttgggattctacaacgacttcaggactgcttgtaagtttctatcccatgtcttttttacagaaagatttattccaatccttacacatcaccaaaggaaatatctgccttgtaaataagtatggcgaggtcctcttctgtgctcaggacagtgaatcttcttttgtattttctctagatctccctaatttaccgcaattccaagcaagaagcccctctgccatagaaattgagaaagcttctggaattcttggtggggagaacctaatcacagtgagtatcaacaagaaacgctacctaggattggtactgaataaaattcctatccaagggacctacactctatctttagttccagtttctgatctcatccaatccgccttgaaagttcctctcaatatttgttttttctatgtacttgctttcctcctcatgtggtggattttctctaagatcaacaccaaacttaacaagcctcttcaagaactgaccttctgtatggaagctgcctggcgaggaaaccataacgtgaggtttgaaccccagccttacggttatgaattcaatgaactaggaaatattttcaattgcactctcctactcttattgaattccattgagaaagcagatatcgattaccattcaggcgaaaaattacaaaaagaattagggattttatcttcactacaaagtgcgttactaagtccggatttccctacgttccctaaagttacctttagttcccaacatctccggagaaggcaactttccggtcattttaatggttggacagttcaagatggtggcgatacccttttagggatcatagggctcgctggcgatattggtcttccttcctatctctatgctttatccgcacggagtctttttcttgcctatgcttcctcggacgtttcgttacaaaaaatcagcaaggatactgccgacagcttctcaaaaacaacagaaggcaatgaggctgtagttgctatgactttcattaaatatgtagaaaaagatcgatctctagagctcctctcgttaagcgagggagctcctaccatgtttctacaacgaggagaatctttcgtacgtctccccttagagactcaccaagctctacagcctggagatcggttgatctgcctcactggaggagaagacatcctcaagtacttttctcagcttcctattgaagagctcttaaaagatcctttaaaccctctaaatacagagaatcttattgattctctaaccatgatgttaaacaacgaaaccgaacattctgcagatggaactctgaccatcctttcattttcataa

SEQ ID NO: 58 - protein, 604 aa, Chlamydia pneumoniae
MKHTFTKRVLFFFFLVIPIPLLLNLMVVGFFSFSAAKANLVQVLHTRATNLSIEFEKKLTIHKLFLDRLANTLALKSYASPSAEPYAQAYNEMMALSNTDFSLCLIDPFDGSVRTKNPGDPFIRYLKQHPEMKKKLSAAVGKAFLLTIPGKPLLHYLILVEDVASWDSTTTSGLLVSFYPMSFLQKDLFQSLHITKGNICLVNKYGEVLFCAQDSESSFVFSLDLPNLPQFQARSPSAIEIEKASGILGGENLITVSINKKRYLGLVLNKIPIQGTYTLSLVPVSDLIQSALKVPLNICFFYVLAFLLMWWIFSKINTKLNKPLQELTFCMEAAWRGNHNVRFEPQPYGYEFNELGNIFNCTLLLLLNSIEKADIDYHSGEKLQKELGILSSLQSALLSPDFPTFPKVTFSSQHLRRRQLSGHFNGWTVQDGGDTLLGIIGLAGDIGLPSYLYALSARSLFLAYASSDVSLQKISKDTADSFSKTTEGNEAVVAMTFIKYVEKDRSLELLSLSEGAPTMFLQRGESFVRLPLETHQALQPGDRLICLTGGEDILKYFSQLPIEELLKDPLNPLNTENLIDSLTMMLNNETEHSADGTLTILSFS

SEQ ID NO: 59 - DNA, 1170 bp, Chlamydia pneumoniae
atgcatccgaaaatagaaaaaagaaatagccttccacttacggcagtcgctcctgtgtttgaagaatcgtatcatccttctgtagctacaactgtagattatgtagatgccacgacactttcccgacatcttacagtcttaaaagatgtgataaaagaagctcgaaacttagatttagggaaggcattcctgacatctatgaaacagggttttataaatacgggtacggaacttgccattatacaagcatctctggcagatcagagtagtcgcgagtcgcgtaagaaggaagagaagatcttccatcagcacttaggaaaggcagccccacaagcggcaacagcaacttcaggagtgcagcctactgcggatcctgttgctgataagatgcctttacaatctgcatttgcctatgttctccttgataagtacattcctgctcaagaggaagccctttatgctcttggaagggagttaaacctatcaggatatgcgcaaaatttatttagtcctcttttagatatgattaagagctttaactctgctcctatcaactacaatttaggatcgtacatatctcagacgagtggcactgcgaatttcgcgtatggttatgagatgattttatcgcgctataacaacgaagtctctcaatgtcgcctggacatagcaagtacagtaaaagctaaagctgcgttagcgaacatgtcggcttctgttaaagcaaatgtgagtctgactgatgcacagaagaaacaaattgaggatatcattgccagctatacgaaatctttagatgtgattcatacacagttaactgatgtgatgacaaatttagcatccataacctttgttcctggtttaaataaatatgatccttcgtatcgcattgttggtggggatttatctatcattgccttgcagaatgacgagaaggtacttgtcgatggtaaggtggatatcacgactgctgtgaatgaaggaggcctacttaatttcttcactacagtccttacggatgtgcagaattatggagacttagctcaaacgcaacagctgatgttggacttagagcttaaggcgatgcaacaacaatggagtttagtatctgcatctttgaaattattgaatgggatgtataccacagtaatttctggatttaaaaactaa

SEQ ID NO: 60 - protein, 389 aa, Chlamydia pneumoniae
MHPKIEKRNSLPLTAVAPVFEESYHPSVATTVDYVDATTLSRHLTVLKDVIKEARNLDLGKAFLTSMKQGFINTGTELAIIQASLADQSSRESRKKEEKIFHQHLGKAAPQAATATSGVQPTADPVADKMPLQSAFAYVLLDKYIPAQEEALYALGRELNLSGYAQNLFSPLLDMIKSFNSAPINYNLGSYISQTSGTANFAYGYEMILSRYNNEVSQCRLDIASTVKAKAALANMSASVKANVSLTDAQKKQIEDIIASYTKSLDVIHTQLTDVMTNLASITFVPGLNKYDPSYRIVGGDLSIIALQNDEKVLVDGKVDITTAVNEGGLLNFFTTVLTDVQNYGDLAQTQQLMLDLELKAMQQQWSLVSASLKLLNGMYTTVISGFKN

SEQ ID NO: 61 - DNA, 537 bp, Chlamydia pneumoniae
gtgacaacaccacaatctcctggttcgctttctcaatctcaccttcctcatccacatgatccttgggatacagaacctacaagtcttcccgaagatccgaatgataaggcaagccaagaactccattcgttagttcatctctttcgcaagctatccattcacctacttagtgaagtcgaaaaaacagtacaacagctaaaacccgacctactagaactggctcttctcatctgtgaaaaatttctctataagaagctagaaaatcctcaagaactggccctgctcctctctaccgcgctccaaagacatacgacattaagatctctgactcccatcaaagtatttctccatcccgaggatctcaaaacacttacagattggatctccacccacgaactccccatgattaagcatgctgagtttttccctgacacttcttgtagacgatcaggattcaaaatagaaacccctaacggaattctgagacaagaaatcagcgaagaactagaccatctactttctgttttgacagcatga

SEQ ID NO: 62 - protein, 178 aa, Chlamydia pneumoniae
VTTPQSPGSLSQSHLPHPHDPWDTEPTSLPEDPNDKASQELHSLVHLFRKLSIHLLSEVEKTVQQLKPDLLELALLICEKFLYKKLENPQELALLLSTALQRHTTLRSLTPIKVFLHPEDLKTLTDWISTHELPMIKHAEFFPDTSCRRSGFKIETPNGILRQEISEELDHLLSVLTA

SEQ ID NO: 63 - DNA, 798 bp, Chlamydia pneumoniae
atgcaaggttttttccctttagcttctggatccaaagggaattctgcttatctaggcacggattcttgtaagattcttattgatttaggagtgagcaagcaagtcgtcactcgggaattactctctatgaatatcgatcctgaagatattcaggcaatttttgttacgcacgaacattctgatcatatctccgggattaaaagttttgttaaggcgtataacactcccattgtttgcaacttggagacggctcgtgctttatgccatctactagatagccatccagaattcaaaatattttccacagggtcttcattttgttttcaagatctcgaagtacagacgttcaatgtacctcatgatgctgtagatcctgtggcttttatttttcattatcgcgaagagaaactgggtttttgcacagatttaggttgggtcacctcttggatcacacatgaactctatgattgtgattacttattaattgagtccaatcattcccctgaattggtacgtcaatctcaacgtcctgatgtttacaaaaagcgtgtattgagtaaattaggtcatatttctaaccaagagtgtggtcagcttttacaaaagattatcactccgaagttaaagaagttatatcttgctcacctgtctaccgagtgtaacactgcggagctagcactttctacagtatctgaatcgatagcttctatcacttctatagcaccagaaattgcattagcgcagggcattacatctccaatatacttttctcgtcttgaggttgcatgtccacggtaa

SEQ ID NO: 64 - protein, 265 aa, Chlamydia pneumoniae
MQGFFPLASGSKGNSAYLGTDSCKILIDLGVSKQVVTRELLSMNIDPEDIQAIFVTHEHSDHISGIKSFVKAYNTPIVCNLETARALCHLLDSHPEFKIFSTGSSFCFQDLEVQTFNVPHDAVDPVAFIFHYREEKLGFCTDLGWVTSWITHELYDCDYLLIESNHSPELVRQSQRPDVYKKRVLSKLGHISNQECGQLLQKIITPKLKKLYLAHLSTECNTAELALSTVSESIASITSIAPEIALAQGITSPIYFSRLEVACPR

SEQ ID NO: 65 - DNA, 429 bp, Chlamydia pneumoniae
atggaagagtttgtagcatatattgttaagaatttagttactaatcctgaagctgttgaaattcgttctattgaagacgaggataacgaatctattaagttagaaattcgtgtcgcagcagaggatattgggaaaattattggaagaagagggaacacaatacatgcgctaagaacgattcttagacgtgtatgttctagattaaaaaagaaagtgcagatagatttggttcagcctgaaaatggaactgatgtgattgctgatcaagattacatctgtgataatgactcttctaactctactgaagatactttcggcgagtctgacacctgctgcagtggacactgtcattacgacgaagatcttaatcaagaagaacaagaagaaggcaatatgcatcattcttgcgaatgtagtaaccaccattaa

SEQ ID NO: 66 - protein, 142 aa, Chlamydia pneumoniae
MEEFVAYIVKNLVTNPEAVEIRSIEDEDNESIKLEIRVAAEDIGKIIGRRGNTIHALRTILRRVCSRLKKKVQIDLVQPENGTDVIADQDYICDNDSSNSTEDTFGESDTCCSGHCHYDEDLNQEEQEEGNMHHSCECSNHH

SEQ ID NO: 67 - DNA, 471 bp, Chlamydia pneumoniae
atggaaaaacaaaatttaaaattagatgtcaaagagattgagtttcctgaaacggtattcagccgtgatatcgaaactcgtgttatccaagtaattattttgcattgtttagcaaaaattaacggtgtttccctcctcggaggaaatctaatagacgctctgttcggtagagatatcgaaagaatgaaggggatctatgtagaacaggattcaaaaaatcatctggtcaaagttcgtgtcgaagtgaacgtagattacggtgtttctataccagagaaaacagaagaaatccagggatgcattgtttcagaaatttcagaatatacaggacttcatgtggccgctgtccacgtgatcattaaagggttgacacaaccaaaagatcgtattgatgaagaaattgaagaggaagtctctgttcaagatcttccttcacctgaagacttcttacttgagaattctgaagggtag

SEQ ID NO: 68 - protein, 156 aa, Chlamydia pneumoniae
MEKQNLKLDVKEIEFPETVFSRDIETRVIQVIILHCLAKINGVSLLGGNLIDALFGRDIERMKGIYVEQDSKNHLVKVRVEVNVDYGVSIPEKTEEIQGCIVSEISEYTGLHVAAVHVIIKGLTQPKDRIDEEIEEEVSVQDLPSPEDFLLENSEG

SEQ ID NO: 69 - DNA, 231 bp, Chlamydia pneumoniae
gtgaaaaataaaattgttacattattagatcagctctacgaggatcaggagtcacgacttcagaagttaggagaggaaattgttcctaacctcactcctgaagatttattgcaacctatggattttcctcaattggaagggaatccggcatttcgttttgaagagggtgtcttatcaggaattggtgaggtgcgagctgcgattttagcggcgctctctcaagagaactag

SEQ ID NO: 70 - protein, 76 aa, Chlamydia pneumoniae
VKNKIVTLLDQLYEDQESRLQKLGEEIVPNLTPEDLLQPMDFPQLEGNPAFRFEEGVLSGIGEVRAAILAALSQEN

SEQ ID NO: 71 - DNA, 522 bp, Chlamydia pneumoniae
atgtggatcatagaccctctatcagcaaaaaaacctctacaagcagccataaatgttcctggcactccaattacaggaggacctaatacagcaactgctgacgatatcattgcaaaattctccaaagactcaaaccctctgattgttactgtttactacgtctatcaatccgtattagttgctcaagataacctctccatcattgcccaagaactccaagcaaactcttcagctcaaacctacctaaataaccaagaagccttataccaatacgtcagtattcctaaaaataaactgaacgataactcctctagctatctacaaaacatccaatccgataaccaagcgataggagcttctcggcaagctatccaaaaccaaatttccagtttaggaaacgcggctcaggtaatctccagtaacttgaacacaaataataacatcatccaacaatccttacaggtaggacaggctcttatccagaccttctctcaaattgtaagcctaattgctaacatctaa

SEQ ID NO: 72 - protein, 173 aa, Chlamydia pneumoniae
MWIIDPLSAKKPLQAAINVPGTPITGGPNTATADDIIAKFSKDSNPLIVTVYYVYQSVLVAQDNLSIIAQELQANSSAQTYLNNQEALYQYVSIPKNKLNDNSSSYLQNIQSDNQAIGASRQAIQNQISSLGNAAQVISSNLNTNNNIIQQSLQVGQALIQTFSQIVSLIANI

SEQ ID NO: 73 - DNA, 222 bp, Chlamydia pneumoniae
ttgtggtataaatctttagctggagaggagaaggacgtgtctgggaatgagtgcaatgactatccagaagtttttaaagatgacgtaagtgcttacgtattggtaacttgtggtcagatgtcttctgaaggcaaaatccaggtggagatgacttatgaaggagatccagctgtgatcagctatttattaacaaaagcacgagactctttagatgagtcttaa

SEQ ID NO: 74 - protein, 73 aa, Chlamydia pneumoniae
LWYKSLAGEEKDVSGNECNDYPEVFKDDVSAYVLVTCGQMSSEGKIQVEMTYEGDPAVISYLLTKARDSLDES

SEQ ID NO: 75 - DNA, 1494 bp, Chlamydia pneumoniae
atgactgtatcttaccaatccatatcgactcctcctcctgaaggagagtttgatatttttgtagatggaaatgccactgaagaagctgttgttgctgcagaagtacaagtagctctccctgcaggagagcaatatgcgatgctgagagcaacttccgagctatgttttgggattttaacacaatcggaatgtgctttgacacaagcactgcctcccaaggaaaaaccattacaagaagagcaatttctagtaaaaaatggcatattaatgcgatcaacatctctgccgaacctaaaaccaggacaatcgcagcagactagcttagcttcacatagaaatcccttagcgcagcaatccacatcttcgaattccacagggaaagcatcaacagaaacaacctcgtcttcctttccgtttttctcatgcaaagctcctgaaggagactccagtgtggacaaaacgttcacagtgtctgtccaaacaccaaaagcacaagagcaacaagaagcaagcgcttctcaaagccaagcacaatttcatgtaaggtcctattctagcagcaccataaaagaacatagtgctaaagaaaaagtctcacaaagcaccaagtcggcagagacacaaaaacacacacaaactaagtctgatgcgactcttagtccgatgtcactctatagcaccttacataaggaagtacctcaagcactatcctctacaaaaagccaacaaaaagatgaagaacatcgagatcaaaggcaacaagaaggatacgagcaagaacaagagcaagaagaaggcaagaaaaagacaccatggtgcactgtggaatctctacaacagacttcaagttcgaaccaggtgtacgagtcttatactcctattattcctgatcctattgtggagtttgctttgtctgaatcacagctaagcgtgcttgcaggaaagcgtgtgaccaaccttgatgtccttagaatatgtacagagctaatgaagttgatgctcaaaagtagagctaacgacacaatgacacgtcttgaagaaagagagctcatggaaagggaagctcatgaattggcggcgagttattcacgtcaagctaaatacgcccgctggctagggatcgcgacagcaacgttaggtattttaggagcgattgctcctatggttggagaaatttccggagatagcattttagggtttgtccaaaggatttctggaagattcaaagatgcgactgcgaaaaccttctttaaaggaataggaaaagttttcacctctctatcacaacttactgaagccgcttctaaagtacatgagttatcagagagtgctgtacgagctgttgctgagtacagaaaagaagtcttcagaatgagacaggatgaagtcacacgtaccattgaggaagtcaaagacaactggaaaagtatggacaatttccttctgaacattctccaaacagaacatgacgctgctcgcagtctgtatcagtag

SEQ ID NO: 76 - protein, 497 aa, Chlamydia pneumoniae
MTVSYQSISTPPPEGEFDIFVDGNATEEAVVAAEVQVALPAGEQYAMLRATSELCFGILTQSECALTQALPPKEKPLQEEQFLVKNGILMRSTSLPNLKPGQSQQTSLASHRNPLAQQSTSSNSTGKASTETTSSSFPFFSCKAPEGDSSVDKTFTVSVQTPKAQEQQEASASQSQAQFHVRSYSSSTIKEHSAKEKVSQSTKSAETQKHTQTKSDATLSPMSLYSTLHKEVPQALSSTKSQQKDEEHRDQRQQEGYEQEQEQEEGKKKTPWCTVESLQQTSSSNQVYESYTPIIPDPIVEFALSESQLSVLAGKRVTNLDVLRICTELMKLMLKSRANDTMTRLEERELMEREAHELAASYSRQAKYARWLGIATATLGILGAIAPMVGEISGDSILGFVQRISGRFKDATAKTFFKGIGKVFTSLSQLTEAASKVHELSESAVRAVAEYRKEVFRMRQDEVTRTIEEVKDNWKSMDNFLLNILQTEHDAARSLYQ

SEQ ID NO: 77 - DNA, 1533 bp, Chlamydia pneumoniae
atgtcctcttggttatctcaagcaagtgaggttcttctcaatcaagatccttatatccctgatgctcctaggtcacaagaatcttcggttcctaagattagctattctatcactgtagcgcctcaagaagctcaaaagtctcttcccaagtttttcactcagaaatttcagtcacaatgtaagtctgagcctcctatcacccatcataaaacattcattattgcaacaccaagagagagaatcttgcgtttcggcagctctttcgaatctcaacttcacaacacatcgcaagctcaaacttcatctccttggaatcttttttctcaaaaaaatagcacagaagcaagtaaagctctgatgcaagaactgactatgccaaaatctccggagaaaacttcagagaaggctctagataaaaacctgagttctaaacaagaaggctcttgcaaaaattttgatacgctgcacctacaacaacatctcaagttgtttggaaccgttgactcgctatattctcaaagcctagatagtgaacagcaagaactcctccaatcacgaagagaagagcgcagtgaaacctatgcaaaccagcagagttctgagaaaaaaatagaaaccaaagttcagataaaagatctctgtaaagacctcttttctcaggatcaggattccaatcaaaaacaaaagaaatcccctttccaacaagatacatcacgtaaaaatagaatagccaaagcagcacaagctgttcctgtaattcctcctccaagcataggagtgtttacattgagctatctactcacaaaacaagggattctttcagacttttcttcgtatggatgccacaaagattccgtggagtcgacacaacgagagctcgatgctctacatgaaaagagaatcgagactatcaaggtcagcatcgagaaagaaaaacgagaaagattatggggatctctttctgacattatcggttggctagctccgtttgtttctatagggatcggcattgttgctatcttgagtggaggcggtatctttgcttttgcaggtttttttgcagggctaatttctcttgttatcaagtgtttagagaaactaaagttctgggattggctagaaaaacatctgcctataaaaaatgaagaactaagacgaaaaattataaccataatccagtgggtcgtctatttaacccctgtcattctctccatatgcacattaaaagtagaaaatttaggtttttcccctattatagaaggagctattaaaggaatccaacccgcgatagaatctacgatggctgctttaagatgcgctatactgttttctcaagcagaaatctataaactcaaaggaaaactcactaaaatccaactcgacattgaattgaaaagttttgatagggacgatcattacgaacgttctcaagaacttttagataacatggagagttctttcgaagccctttcaagaatcttaaattacatgcgtgaactagatcaagtgtatctccactccttaagaggataa

SEQ ID NO: 78 - protein, 510 aa, Chlamydia pneumoniae
MSSWLSQASEVLLNQDPYIPDAPRSQESSVPKISYSITVAPQEAQKSLPKFFTQKFQSQCKSEPPITHHKTFIIATPRERILRFGSSFESQLHNTSQAQTSSPWNLFSQKNSTEASKALMQELTMPKSPEKTSEKALDKNLSSKQEGSCKNFDTLHLQQHLKLFGTVDSLYSQSLDSEQQELLQSRREERSETYANQQSSEKKIETKVQIKDLCKDLFSQDQDSNQKQKKSPFQQDTSRKNRIAKAAQAVPVIPPPSIGVFTLSYLLTKQGILSDFSSYGCHKDSVESTQRELDALHEKRIETIKVSIEKEKRERLWGSLSDIIGWLAPFVSIGIGIVAILSGGGIFAFAGFFAGLISLVIKCLEKLKFWDWLEKHLPIKNEELRRKIITIIQWVVYLTPVILSICTLKVENLGFSPIIEGAIKGIQPAIESTMAALRCAILFSQAEIYKLKGKLTKIQLDIELKSFDRDDHYERSQELLDNMESSFEALSRILNYMRELDQVYLHSLRG

SEQ ID NO: 79 - DNA, 588 bp, Chlamydia pneumoniae
atggcttacggaactcgttatcccacgctagcattccatacagggggcattggtgaatctgatgacggtatgcccccacaaccctttgaaactttctgctacgattctgctcttctacaagcaaaaatcgaaaattttaatatcgtcccttatacatctgtacttcctaaagagctctttgggaatatcgttcctgtagatacctgtgtaaaatctttcaaacacggcgctgtccttgaagtaatcatggcgggtcgtggtgctgctctctctgacggaacccatgcgatcgccaccggaatcggcatttgctggggtaaagataaaaatggagagcttattggcggatgggcagcagaatatgttgaattcttccctacatggataaatgacgaaattgcagaaacccatgctaaaatgtggttgaaaaaatccctacaacacgaacttgacctacgctctatagcaaagcatagcgaattccaattcttccataattacatcaacatcaaacaaaaattcggtttttgcttaactgcattaggattcctaaatttcgaaaatgctgaaccagctaaggtaaattaa

SEQ ID NO: 80 - protein, 195 aa, Chlamydia pneumoniae
MAYGTRYPTLAFHTGGIGESDDGMPPQPFETFCYDSALLQAKIENFNIVPYTSVLPKELFGNIVPVDTCVKSFKHGAVLEVIMAGRGAALSDGTHAIATGIGICWGKDKNGELIGGWAAEYVEFFPTWINDEIAETHAKMWLKKSLQHELDLRSIAKHSEFQFFHNYINIKQKFGFCLTALGFLNFENAEPAKVN

SEQ ID NO: 81 - DNA, 1092 bp, Chlamydia pneumoniae
atgaaattatatcagaccttgcgaggtattgttttagtaagtacgggatgcatattcttaggaatgcacggaggatatgccgctgaggttccagtgacttcatctgggtatgagaatcttttagaatctaaggaacaggatccttcaggtctagcgatccacgatcgcattttgtttaaggtagatgaagagaatgtagtgactgccttagatgtgatccataaattaaacttactattttataattcgtatcctcatcttatagattctttccctgcacgatcccagtactatactgcgatgtggcctgtggttcttgaatctgtgattgatgagtttttgatggtggcagatgccaaggcaaagagaatcgctacagatcccaccgcagtgaatcaagaaatcgaagagatgttcggaagagatctctctcctttgtatgcgcattttgaaatgagtcccaacgatatttttaatgtgatcgatcgcactttgacagcacaaagggtgatgggtatgatggtgcgctctaaggtaatgttgaaggtgactccagggaaaattcgagaatattaccgaaagctagaagaagaagcctctaggaaagtcatctggaagtatcgtgtgttgacgattaaagccaacacagaatccttggctagccagattgctgataaagtgcgtgctcgtctaaatgaagcgaaaacctgggataaagatcgtttaactgctcttgtgatctctcagggagggcaactcgtctgctccgaagagttttctcgagagaatagtgagctctcccaaagccataagcaagagctggacttgattggctatcctaaagagctctgtgggttgcctaaggcacataagtcaggatataaactctatatgttgttagacaaaacctcaggttctatagagcctttagatgttatggagtccaagatcaaacagcatctttttgctttagaagctgagagtgtagagaaacaatataaagacagattacgcaagcgctacggctatgatgcttctatgattgcgaaacttctttctgaagaagctccacctctattttccttattatag

SEQ ID NO: 82 - protein, 363 aa, Chlamydia pneumoniae
MKLYQTLRGIVLVSTGCIFLGMHGGYAAEVPVTSSGYENLLESKEQDPSGLAIHDRILFKVDEENVVTALDVIHKLNLLFYNSYPHLIDSFPARSQYYTAMWPVVLESVIDEFLMVADAKAKRIATDPTAVNQEIEEMFGRDLSPLYAHFEMSPNDIFNVIDRTLTAQRVMGMMVRSKVMLKVTPGKIREYYRKLEEEASRKVIWKYRVLTIKANTESLASQIADKVRARLNEAKTWDKDRLTALVISQGGQLVCSEEFSRENSELSQSHKQELDLIGYPKELCGLPKAHKSGYKLYMLLDKTSGSIEPLDVMESKIKQHLFALEAESVEKQYKDRLRKRYGYDASMIAKLLSEEAPPLFSLL

SEQ ID NO: 83 - DNA, 2076 bp, Chlamydia trachomatis
atgacgctctttcattctcatcatgatgccgtctctccagacagctacctatgttcttcccttcagttagttggtactggcgtatacgaaggagaaatcgagattcaaaatatcccctcttatttccttggattccaattaccctctcattgcatacaccttaatttaaagagctctctagctcaattaggaatagatgcctcccttcttcactgcgaattgagcaaaaatcaacatcgagcacatatacatgctcaatttaccggtcatggccccattgctgaatctatgctagcccttctccaaccaggagatcgtgtagcaaaactatttgctgcagacgatcgcagactggtccgatctccagattacctcgaaagcatgctgaaaaatacagataaagctggccatcctttgctctgttttgggaaaaaattagaacacttgatttcttttgatgtggtagatgatcgccttgtcgtctcccttcctaccctgccgggagttgttcgttatgattcggatatttatggactccttcctcttattcaaaaatcactcagtaatcccaaactcagcattcgtcactttttagctctgtaccaacagattgtggaagggcaacatgtctcttgcggaaaccatattcttctgatcaaaacagaaccgctgcacatccgcactgtatttgctcgcgtggtaaatcaactcctccctcaaggtctctcccacacttctgccaatattttggaaccaaccactcgagaatccggggatatctttgaattttttgggaacccttctgcacagatagaaagaattcctttagaatttttcactatcgaaccctataaagaacattcttacttctgtaatcgggatttattacaaaccatcttacaatcagaaagcgaaatcaaaaaaatattcgaaacagcgcccaaagaacctgtcaaagctgccacctatttatcaaaaggcagtgaaatctcttccctgcacacagactcttggctcacaggatccgcagctgcctatcaatatagtgagcaagcagataaaaacgagtacactcatgctcaaccttgctatcctttcttagaagcaatggaaatgggcctgatcaatagcgaaggagccttactcactcgttatttcccttcagctagcttaaaaggaatgttgatttcctaccatgtgcgccactatctcaaacaaatctactttcaagttccctcttatacacatggaaactatttctctcataatgacagaggtttgctattagatctgcagcaagcagatattgatgttttctgggcagatgaagaaagcggccgtgtgttgcaatatacaaaacgacgcgataagaatagcggtatgttcgtgatcaaaaatcgtgttgaagagtttcgatcagcttattttattgctatttatggctctcgtctccttgagaataatttctctgctcagctccataccctcctagcgggcttacagcaagcagcacatactctcggcattcctggattctcaaagcctaccccacttgcagtcatcaccggaggcggcactggagttatggccacaggaaatcgtgtagctaaagaactaggaatcctatcttgtggaaccgttcttgatttagaagcttctccagcacaaatcgaccaacctaccaatgaattcttagatgctaaaatgacataccgcctacctcaacttatagaaaggcaagaacacttttatgcagaccttcctatccttgtagttggcggtgtaggaaccgatttcgaactctacctagaacttgtctatctcaaaacaggagctaaaccaccgactcccattttcctaattggacctattgaatactggaaagaaaaagtggcccacgcctacgagatcaacctcaaagcaggaaccatccgtggatccgaatggatcagcaactgcctatattgtatcacttctccggaagctggaattgccgtattcgaacaattcctagctggagaactccctataggatacgactatcctccagctccagatggattagtgatcgtctaa

SEQ ID NO: 84 - protein, 691 aa, Chlamydia trachomatis
MTLFHSHHDAVSPDSYLCSSLQLVGTGVYEGEIEIQNIPSYFLGFQLPSHCIHLNLKSSLAQLGIDASLLHCELSKNQHRAHIHAQFTGHGPIAESMLALLQPGDRVAKLFAADDRRLVRSPDYLESMLKNTDKAGHPLLCFGKKLEHLISFDVVDDRLVVSLPTLPGVVRYDSDIYGLLPLIQKSLSNPKLSIRHFLALYQQIVEGQHVSCGNHILLIKTEPLHIRTVFARVVNQLLPQGLSHTSANILEPTTRESGDIFEFFGNPSAQIERIPLEFFTIEPYKEHSYFCNRDLLQTILQSESEIKKIFETAPKEPVKAATYLSKGSEISSLHTDSWLTGSAAAYQYSEQADKNEYTHAQPCYPFLEAMEMGLINSEGALLTRYFPSASLKGMLISYHVRHYLKQIYFQVPSYTHGNYFSHNDRGLLLDLQQADIDVFWADEESGRVLQYTKRRDKNSGMFVIKNRVEEFRSAYFIAIYGSRLLENNFSAQLHTLLAGLQQAAHTLGIPGFSKPTPLAVITGGGTGVMATGNRVAKELGILSCGTVLDLEASPAQIDQPTNEFLDAKMTYRLPQLIERQEHFYADLPILVVGGVGTDFELYLELVYLKTGAKPPTPIFLIGPIEYWKEKVAHAYEINLKAGTIRGSEWISNCLYCITSPEAGIAVFEQFLAGELPIGYDYPPAPDGLVIV

SEQ ID NO: 85 - DNA, 966 bp, Chlamydia trachomatis
atgaagcgtttattttttatctgcgccctcgccctttctcctctagcatatggagctgttcaaaaggatcctatgttaatgaaggagactttccgtaataactacgggatcattgtctctaagcaagaatggaacaaacgtggatgcgatggctccatcactagagtattcaaagatggaactacaaccttagaagtttatgcgcaaggtgctttacatggggaagtcacacgaacgtttcctcactctactaccctggccgttatagaaacttatgatcagggaaggcttctttctaagaagaccttcttcccaaatgctttgcctgctaaagaagaagtttaccacgaagatgggtctttctccctaacacgttggcctgacaataacaactctgacacaatcacagacccctgctttgtagaaaaaacttatgggggaagagtattggaaggtcattacacctcttttaatggaaaatactcttcaacaatccttaacggcgagggagttcgctctactttttcttcggatagtatcttgttgacagaagagtcgtttaatgatggcgtaatggtcaaaaaaacgacattttactcgactcgagaacccgaaaccgtcactcattatgtcaatgggtaccctcacggagttcggtttacctatcttcctggtgggattccaaatacgattgaagaatggcgatatggacatcaagacggccttacaatcttatttaaaaatggttgtaagattgctgaagtcccatttgtacgcggagcaaaaaatggaatcgaactccgatacaatgaacaagagaatatcgctgaagagatttcttggcagcacaacatcttgcatggagtccgtaaaatccatgcggcgggggtatgcaaatccgaatggtattacaaaggcaaacctgtctcgcaaatcaagtttgaacgactcagcgctgccagataa

SEQ ID NO: 86 - protein, 321 aa, Chlamydia trachomatis
MKRLFFICALALSPLAYGAVQKDPMLMKETFRNNYGIIVSKQEWNKRGCDGSITRVFKDGTTTLEVYAQGALHGEVTRTFPHSTTLAVIETYDQGRLLSKKTFFPNALPAKEEVYHEDGSFSLTRWPDNNNSDTITDPCFVEKTYGGRVLEGHYTSFNGKYSSTILNGEGVRSTFSSDSILLTEESFNDGVMVKKTTFYSTREPETVTHYVNGYPHGVRFTYLPGGIPNTIEEWRYGHQDGLTILFKNGCKIAEVPFVRGAKNGIELRYNEQENIAEEISWQHNILHGVRKIHAAGVCKSEWYYKGKPVSQIKFERLSAAR

SEQ ID NO: 87 - DNA, 426 bp, Chlamydia trachomatis
gtgagtttagattttttagaggattttttccgtcgctcaattaccaatcacaacacagcttttccagaaggctttctggatatatctgatgtcttagctcgttcggctttagattttaaggctgaggaacttgctgacagtgctgttaatgacttcatcgtatcagaatcttcagataaactcactttatttaacacaaattttgctgtgtggttggtacctacattagttgatggtgaggcaattactcgcggctacatcgctttaaaccagggtgaagagttctctcctgaattagcttttgaagcatcaggaaagtataataattcgagtttaatcttagaggcgttgcgaagatatttgtgtgatatccaggatacagaaaaagaattacgggcattacgcccgccttcaatagatggatag

SEQ ID NO: 88 - protein, 141 aa, Chlamydia trachomatis
VSLDFLEDFFRRSITNHNTAFPEGFLDISDVLARSALDFKAEELADSAVNDFIVSESSDKLTLFNTNFAVWLVPTLVDGEAITRGYIALNQGEEFSPELAFEASGKYNNSSLILEALRRYLCDIQDTEKELRALRPPSIDG

SEQ ID NO: 89 - DNA, 240 bp, Chlamydia trachomatis
ttggaagatagaatgatcgacgggattcagacgtgttcttttagccctacgcatcgcttaactgcgaaatccgcagtgagtatagagatgcctttagcaacacaaaatcttcaagagggagctttggtcaatgcaaagctcgaagcggatttcgcgagagcagagcagattcttacagagatgcaagaaatccgttctagtttagagaggtctttagagactctctttccccgcgagtaa

SEQ ID NO: 90 - protein, 79 aa, Chlamydia trachomatis
LEDRMIDGIQTCSFSPTHRLTAKSAVSIEMPLATQNLQEGALVNAKLEADFARAEQILTEMQEIRSSLERSLETLFPRE

SEQ ID NO: 91 - DNA, 696 bp, Chlamydia trachomatis
atgatggaggtgtttatgaattttttagatcagttagatttaattattcaaaataagcatatgctagaacacacattttatgtgaaatggtcgaagggggagcttactaaagagcaattacaggcgtatgccaaagactattatttacatatcaaagcctttcctaaatatttatctgcgattcatagtcgttgcgatgatttagaggcgcgtaagttattgttagataacttgatggatgaagagaacggttaccctaatcatattgatttgtggaagcagtttgtgtttgctctaggagttactccagaagagttagaggctcatgagcctagtgaagcagcaaaagcgaaagtagctactttcatgcggtggtgtacaggagattctttagctgcaggagtggctgctttgtattcttatgagagtcaaattccacgtatcgctagagagaaaattcgtggattgactgagtactttggattttccaatcctgaagactatgcatatttcacagaacatgaagaagcggatgtgcggcatgctagagaagaaaaagcgctcattgagatgcttctcaaagatgacgctgataaagtgttagaggcatcgcaagaagtaacgcaatctttgtatggctttttagattcttttttggatccaggaacttgttgtagttgtcatcaatcttattaa

SEQ ID NO: 92 - protein, 231 aa, Chlamydia trachomatis
MMEVFMNFLDQLDLIIQNKHMLEHTFYVKWSKGELTKEQLQAYAKDYYLHIKAFPKYLSAIHSRCDDLEARKLLLDNLMDEENGYPNHIDLWKQFVFALGVTPEELEAHEPSEAAKAKVATFMRWCTGDSLAAGVAALYSYESQIPRIAREKIRGLTEYFGFSNPEDYAYFTEHEEADVRHAREEKALIEMLLKDDADKVLEASQEVTQSLYGFLDSFLDPGTCCSCHQSY

SEQ ID NO: 93 - DNA, 816 bp, Chlamydia trachomatis
ttgagagcagccttgaatacacttgaatttctttcttctcccccctcttcggatccttacgatgatttattacaattaaacaaagaaggattccttgctggccctgaagaagaaaaacaagctttttttcttcgggtagaaaggacattagcagaagctcctgtacatcccaccccttttcccatagaattacagaaactcttcgatgtgaacccttcttttttagaggttgtgtactctaatgaaagtttagatgcctgggaagcaggatgtacatggatcaccgataacagagtatcgattcaactacgcaaacgttttcaaaaagcttctttctggtttggttttttttccaaagaagaagtgctgtctcacgaagctgttcatgctgtgcgtatgaaattttatgaaccgatctttgaagaggtcttggcatacagcacttctaaacacttttggagacgcttttttggtcccctgttccgatcagcagaagaaacgcatttctttctgtttttcgttttatttggagcgtttttattcccttggtttccttggataggcctttcttgtattcttgctcctaatatgttctttttttttcgcttattccgaacacaaatcctatttcgtaaagcaaagaaaaaaattcgaaaacttttaggtatagaacctctctgggtcttactacgcttaacagatagagaaattcgcctatttgctacacagcccttagctgtgatagaagacttcgctaggaaagaaaagctgaaaagtgtgcgctggaggcaaatctatcaaagttacttcacctaa

SEQ ID NO: 94 - protein, 271 aa, Chlamydia trachomatis
LRAALNTLEFLSSPPSSDPYDDLLQLNKEGFLAGPEEEKQAFFLRVERTLAEAPVHPTPFPIELQKLFDVNPSFLEVVYSNESLDAWEAGCTWITDNRVSIQLRKRFQKASFWFGFFSKEEVLSHEAVHAVRMKFYEPIFEEVLAYSTSKHFWRRFFGPLFRSAEETHFFLFFVLFGAFLFPWFPWIGLSCILAPNMFFFFRLFRTQILFRKAKKKIRKLLGIEPLWVLLRLTDREIRLFATQPLAVIEDFARKEKLKSVRWRQIYQSYFT

SEQ ID NO: 95 - DNA, 180 bp, Chlamydia trachomatis
atggaccagttatcacagatacatcaagagctagctcgtttagaatttatcaatgatcaactgcagtcggaaagagcgtacatccatgatttattgtgtgcgataggctttcctgaagggttaaagacgatagccgcgatagctaacgaagtgctttccgaagaagattcccaaggttaa

SEQ ID NO: 96 - protein, 59 aa, Chlamydia trachomatis
MDQLSQIHQELARLEFINDQLQSERAYIHDLLCAIGFPEGLKTIAAIANEVLSEEDSQG

SEQ ID NO: 97 - DNA, 2490 bp, Chlamydia trachomatis
atgggtatacgcttagttattgataaagggcccttgtctggaactgttcttattttagaaaatgggacgagttggtctcttggcagtgatggaaaagctagtgatattctcctgcaagatgaaaagcttgctccctctcagattcgcatcactttaaaagatggcgagtattatttagaaaatttagatgctttgaggccggtttctgttgatggaacagttatcactgcccctgttttgttaaaagatggggtttcctttgtaatgggaagctgccaagtctcgttttttaaaggggaagaggtagaaggagatatagagttatcgttccagacagaaggtggtaatgagggagagcctgcagcgcaaggctcttcaagcgtttcgtccgaagctcctaaaaaggagacagggaatccaagtcttccttcggaggcaaaggcttctggagaagtatctagttcagcaatagcgaaagaacaagagttagcggcgtcctttttagcttctgttgagaaggagcctggaacaccaaaagaagtctctgagccaaaggtctcttcacaagaaggacagactccttctgttacaggagaaaaaaaggatcttgagcttcctttggcaagtcaagaacaacctaaacaaactactccatcaggcagtggtgaaccaacccaatctcaaaacgcgagtatggaagaaaacagaacgtcgcccgatcaaaatcagcagccacagctttcttctgcttcagaatcgggttctcaaagtcccgaaaatcaggagcaacaaccttctcaaacgcctcccccatccccggaaactccagagccgtcaggagaacctaatagcgctacggaagaaaactcgccatctccaatggagaaagcttccgtaacagaagaaggcagctcagggacgagtgaagaagaaaaagagggtgaagaagatactgctgaaagcgcagcaaatgaagagccaaaggcagaggcttctcaagaagaagagaagaaagaggaagataaaggagaggttcttgctccctttaatgttcaggatcttttccgttttgatcaaggaatcttccctgctgagatagaagatcttgcacagaaacaagttgcggttgatttgacgcaaccatcacgatttttgttgaaggttcttgctggtgcgaatatcggtgctgaattccatttggatagtgggaaaacctatatcgtaggaagtgatccgcaggttgcagacattgtcttaagtgatatgagtatttcgcgccaacatgcgaagatcattatcggaaatgataattcagttttgattgaagatctgggtagtaagaatggcgtgattgttgaagggcgcaagattgaacatcaatctacgctctctgcgaatcaagttgttgctctaggaacaacgttattcttacttgtcgactatgctgctccttccgatacggtaatggcgacgatttcttctgaagattatgggttatttggtcgtccgcaatctcctgaagagattgctgccagagctgcggaagaggaagaagagaagagaaaacgtgctacgttgccaacaggtgcttttatattaaccttgttcattggagggttagctctgctctttggaataggaacagcttctttgttccatacgaaggaagtagtttctatagatcaaatcgatttgattcatgatattgaacatgtaattcagcagtttccaactgtacggtttacgttcaataagaacaacggacagttgttcttaattgggcatgtaagaaatagcattgataagagcgagttactttacaaagtggatgctctctcgtttgtcaagtcggtagatgataacgtgatcgatgacgaggcagtatggcaagagatgaatattctcttgtctaagaatccagaatttaaaggtatcagcatgcaatctccagagccggggatttttgtaatcagcgggtatctaaagacagaagaacaagcagcttgtttggctgattatctaaatctacattttaattacctttcactattggataataaggtgattatcgaatcacaagtcatgaaagctcttgctggacatcttgtgcaatcaggttttgcgaacgttcatgtgtccttcaccaatggtgaagctgttttgacaggatatatcaataataaagatgcagataaattccgaacggttgtgcaagaactgcaagatattgcagggattcgtgcggtgaagaattttgtcgttttgctgcctgcagaagaaggtgttattgatctaaatatgcggtatccaggccgttatcgggtaaccggtttttcaaagtgcggggatattagtattaatgttgtagttaatgggcgtattttaactcgaggcgatattttagatggaatgacggtaacaagcattcaatcgcattgtatctttttagaacgggaagggttgaaatataaaattgagtacaataaatag

SEQ ID NO: 98 - protein, 829 aa, Chlamydia trachomatis
MGIRLVIDKGPLSGTVLILENGTSWSLGSDGKASDILLQDEKLAPSQIRITLKDGEYYLENLDALRPVSVDGTVITAPVLLKDGVSFVMGSCQVSFFKGEEVEGDIELSFQTEGGNEGEPAAQGSSSVSSEAPKKETGNPSLPSEAKASGEVSSSAIAKEQELAASFLASVEKEPGTPKEVSEPKVSSQEGQTPSVTGEKKDLELPLASQEQPKQTTPSGSGEPTQSQNASMEENRTSPDQNQQPQLSSASESGSQSPENQEQQPSQTPPPSPETPEPSGEPNSATEENSPSPMEKASVTEEGSSGTSEEEKEGEEDTAESAANEEPKAEASQEEEKKEEDKGEVLAPFNVQDLFRFDQGIFPAEIEDLAQKQVAVDLTQPSRFLLKVLAGANIGAEFHLDSGKTYIVGSDPQVADIVLSDMSISRQHAKIIIGNDNSVLIEDLGSKNGVIVEGRKIEHQSTLSANQVVALGTTLFLLVDYAAPSDTVMATISSEDYGLFGRPQSPEEIAARAAEEEEEKRKRATLPTGAFILTLFIGGLALLFGIGTASLFHTKEVVSIDQIDLIHDIEHVIQQFPTVRFTFNKNNGQLFLIGHVRNSIDKSELLYKVDALSFVKSVDDNVIDDEAVWQEMNILLSKNPEFKGISMQSPEPGIFVISGYLKTEEQAACLADYLNLHFNYLSLLDNKVIIESQVMKALAGHLVQSGFANVHVSFTNGEAVLTGYINNKDADKFRTVVQELQDIAGIRAVKNFVVLLPAEEGVIDLNMRYPGRYRVTGFSKCGDISINVVVNGRILTRGDILDGMTVTSIQSHCIFLEREGLKYKIEYNK

SEQ ID NO: 99 - DNA, 525 bp, Chlamydia trachomatis
ttggaggattacgtggcttctcctcatcttcgttccctagcgtgtttagataacccacaactacccatagaaacacctctctttgagcaagaagctctctcccatgagcttctttctcttattcaggtgttccgtaaattatctgtccatcttctctctgaaatcgaaaaattatctcagaaactaaaacctgagcttcttgaacttgctgtcctcgtctgtgaaaaatttctgtacagaaagcttgcctgtacagaagaacttgctctcctaatctccgcagctctgcaacatcatttagctacttatgccgtctctcccataaaaataggtttacatcctgaagatctttcaaacctatctaaatggttaatccttcacgatgttcccttactcaaaaatatcgaattcattgcagatcctttatgcaagaaagctagctataaaatagaactcccttcaggaattctgagacaagacatcggggaagagctgtcccatctactgagtgtactcactccataa

SEQ ID NO: 100 - protein, 174 aa, Chlamydia trachomatis
LEDYVASPHLRSLACLDNPQLPIETPLFEQEALSHELLSLIQVFRKLSVHLLSEIEKLSQKLKPELLELAVLVCEKFLYRKLACTEELALLISAALQHHLATYAVSPIKIGLHPEDLSNLSKWLILHDVPLLKNIEFIADPLCKKASYKIELPSGILRQDIGEELSHLLSVLTP

SEQ ID NO: 101 - DNA, 420 bp, Chlamydia trachomatis
atggaagattttgcagagtacatcgttaaaaatttagttaccgatcccaacgccgttgagattcggtcatccgaggacaaagccagcgcaacccttaagctggagatccatgccgcttctgaagatattggaaagatcatcgggagaaaaggacaaaccatacaagcgctaagaaccattctaaaacgtgtaggcgctagattgcagaaaaaaatccttgttgagcttgctcaacctgaaaacggctctctcacagacgaagaagttttgtctttagattatatctctgcagcttctgccgaagatttcgaagaagattcttcttttgctgaaaatagcatcgaagaagaaccttcggtcattgtacgatctcttgcaggtgtatgcccaggttgtagctgctctcatcatcatgattag

SEQ ID NO: 102 - protein, 139 aa, Chlamydia trachomatis
MEDFAEYIVKNLVTDPNAVEIRSSEDKASATLKLEIHAASEDIGKIIGRKGQTIQALRTILKRVGARLQKKILVELAQPENGSLTDEEVLSLDYISAASAEDFEEDSSFAENSIEEEPSVIVRSLAGVCPGCSCSHHHD

SEQ ID NO: 103 - DNA, 279 bp, Chlamydia trachomatis
atgaaggaagaaattctcgcgctacttgatcatttatatacggagcaggaaagacgattaatgtcgctagggacgacgattgttcctggattgacgaaagaggatcttttacagcctatggattatgatgaacttgaggagaacccttcttttagatttgaagaaggagttttgaatggaataggagagactcgagccgcattatattcttttttttctgatctagaagactccttttgcgtggagtcttctagcgatacgagcctctgtaaggattag

SEQ ID NO: 104 - protein, 92 aa, Chlamydia trachomatis
MKEEILALLDHLYTEQERRLMSLGTTIVPGLTKEDLLQPMDYDELEENPSFRFEEGVLNGIGETRAALYSFFSDLEDSFCVESSSDTSLCKD

SEQ ID NO: 105 - DNA, 507 bp, Chlamydia trachomatis
atgtggcataaagaaccaatgcatgctgtattacagctacctgaaacacctctggttacaggaacaacgaactctgcaacggctgatgagattataacacgatttgctaaggattctaaccctctcatcgttactgtttactatatttaccagtctgttctcgtcgcgcaaaataacttgtctctagttgcagagcaattgcaagctaatgctgccgcgcaaacattcctgaacaatgaagaagcgctataccaatacactaccattccaaaaaaccaagtaaactctcaaaactcctcttatttacaaaacgtacaatcggttaaccaagcagtcggagcatctagacaagcgattcaaaaccaaatatctggtcttggaaatgcttctcaagtgatttccagtaacttgaacacgaataataatattattcaacagtccctacaagtcggtcaggcgctcattcaaacgttttcacaaatcgttagcttgatcgcgaatatctaa

SEQ ID NO: 106 - protein, 168 aa, Chlamydia trachomatis
MWHKEPMHAVLQLPETPLVTGTTNSATADEIITRFAKDSNPLIVTVYYIYQSVLVAQNNLSLVAEQLQANAAAQTFLNNEEALYQYTTIPKNQVNSQNSSYLQNVQSVNQAVGASRQAIQNQISGLGNASQVISSNLNTNNNIIQQSLQVGQALIQTFSQIVSLIANI

SEQ ID NO: 107 - DNA, 486 bp, Chlamydia psittaci
gtgcgcatactaccctttgatccttatggagcattacctcctcaaggagtaaataaagatcctcatagcaatatacctttaaatcagaagatttctgatgaaatagctaaaaatgaagctatgcgtttggctttgcttgctattactgatcaagaaaaagacaaaagaaagagaaaacatcggtttaaaattcttaaccgcaaacaagcaaaagtgctcctatctcaattgcgaaatgtagatttagactttcaaagtttgaaaaatgctggtttaaaagaagaagaagataacgaagaagataatgaagaatcttcaaaacaagggaaaactttccatattgctagtagtaagaagcctgtgaagataggggcatctgcagctcaggctattgctgatgctgcagaggcttgggttattgctcgcaatagaggagtcttggatatggcctccctattattctggcataaagatgatgagtgttaa

SEQ ID NO: 108 - protein, 161 aa, Chlamydia psittaci
VRILPFDPYGALPPQGVNKDPHSNIPLNQKISDEIAKNEAMRLALLAITDQEKDKRKRKHRFKILNRKQAKVLLSQLRNVDLDFQSLKNAGLKEEEDNEEDNEESSKQGKTFHIASSKKPVKIGASAAQAIADAAEAWVIARNRGVLDMASLLFWHKDDEC

SEQ ID NO: 109 - DNA, 447 bp, Chlamydia psittaci
atgaaaagtgaacgtctgaaaaaactcgaatctgaattacgtgacttaacccagtggatgcaattgggtttagttccaaaaaaagaaatcgatagacataaggaagaaatacgttctttagaaaacaaaatccatgaagagaaggaacgcctgcaacttctaaaagaaagtggcgaagttgaagagtttgttactccaagacgtagccctgcaaaaacggtatatcctgacggtccaagcatgtcagatatggaatttgttgaagctacagagacagaaattgatattgatccaggtgaaaccgtagagctcgaacttcctgatgaagaacgtgaagaaggcgctgtagaaatcgattattccagtgatgacgatgaagatcctttcagtgatcgcaatcgttggagacgtgggggcattgttgaccccgatgctaatgaatggtaa

SEQ ID NO: 110 - protein, 148 aa, Chlamydia psittaci
MKSERLKKLESELRDLTQWMQLGLVPKKEIDRHKEEIRSLENKIHEEKERLQLLKESGEVEEFVTPRRSPAKTVYPDGPSMSDMEFVEATETEIDIDPGETVELELPDEEREEGAVEIDYSSDDDEDPFSDRNRWRRGGIVDPDANEW

SEQ ID NO: 111 - DNA, 972 bp, Chlamydia psittaci
atgaaacggctgcttttttgcgtttgcgcactctctttctcatgctttacctacggttcagctctaaaacaagattcttctgttatgaaggaaaccttccgtaacaactacggaattattgtatcaggaaaagattgggtaaaacgaggctgtgatgggacaatcaccaaagttttaaaggatgggtctactctttatgaaatttacgttcagggtcttcttcatggcgagataacgttaacattcccccattctacgactcttgctgtaataaaaacttacgatcagggaaggcttgtttcatataaaacattcttttctaatggtttaccttctcaagaagaattataccaagaagatggctctcttgttgtgactcgctggcctgacaacaaaaatagcgataccatcaccgatccttattttactgagactacctaccagggtcgtgtacttgaagggagctactcctcatttaatgggaaatatacatcaacaattcgcaatggagaaggcatacgctcgaacttttctccaagtaacgtccttctttctgaagaaacctttaacgatggcgttatggtaaaaagaactaccttctatgctactagagatcctgaaactataacgcattatattaatggccaacctcatggattgcgtttaacctatctccctggagggattcctaacactattgaagaatggcgttatggttaccaagatggaactacaacagtatttaaaaacggttgcaaagctgctgagattccttttgtaaaaggatctaaggaaggatgtgaattacgctataatgaagacgaagttattgccgaagaagtgtcttggagaaataacttccctcatggtatgagaaagatctatgctgccggtgtttataaatgcgagtggtaccaccgcggacgcctagtctcaaaagctaagttcgagagactcaataatgcaggataa

SEQ ID NO: 112 - protein, 323 aa, Chlamydia psittaci
MKRLLFCVCALSFSCFTYGSALKQDSSVMKETFRNNYGIIVSGKDWVKRGCDGTITKVLKDGSTLYEIYVQGLLHGEITLTFPHSTTLAVIKTYDQGRLVSYKTFFSNGLPSQEELYQEDGSLVVTRWPDNKNSDTITDPYFTETTYQGRVLEGSYSSFNGKYTSTIRNGEGIRSNFSPSNVLLSEETFNDGVMVKRTTFYATRDPETITHYINGQPHGLRLTYLPGGIPNTIEEWRYGYQDGTTTVFKNGCKAAEIPFVKGSKEGCELRYNEDEVIAEEVSWRNNFPHGMRKIYAAGVYKCEWYHRGRLVSKAKFERLNNAG

SEQ ID NO: 113 - DNA, 975 bp, Chlamydia psittaci
atgacatctgcaatgcctcgtgtggctagccttgtagtgggcagtagaaatgtgtttatgcaaacagctatgcagggagccaagaaaggcgatgtaggctgctgcattaggcagtttgttacgaatggaaacaaccatttagcacgctttgtcggaagtacaaaaaatatagataaggcatttaagtttgctaaatccgtctctgaatttagctgcggtgttattgaaagcacaggtgatacaggacctgctcttcagataagtaaaaacgttgcatcgactttaagcacggctagaaatgttgtcgccttgtctaatgtgttcactggtgcgattcctggattcgtcctttcctcaaagaattgctataatcatattaagaaatgttttgctccagaaactgagtacgattgcggtagtattgagaaaggcttgccttataacaagctttatctcactaagggtgatcatgtgttgggtgcgattaaagaaggttgctctgcagttggcgcgggaacttacgttgcaacgtttggtgtcagccgtccggtgctcttggcaaacaagcttgctcacaagcctttcctctcttcaggagtgaaggcagcattttgtaacagcgtaacctatatgatgacagcaaaccacgctgcaggggttatcggcggagcagcagccttgctctatgaaaaccgtgtatataaacgcgcttctgagggattgttagcttctaaaatgacagagagtctggattctgaagtttacgatcaagtgtctaaaggcttgaaagagtcgcactaccaagctgttaaaaaaacaatacttggaattttagaaaaagcatttgaattgatcgctgatatattcgatttcattgctttaccaattactgcttcagttcgcttggcaataaaagcgggattagtcacagtatccagtggtttcggtctttacagcgtctgggtcaattcttaa

SEQ ID NO: 114 - protein, 324 aa, Chlamydia psittaci
MTSAMPRVASLVVGSRNVFMQTAMQGAKKGDVGCCIRQFVTNGNNHLARFVGSTKNIDKAFKFAKSVSEFSCGVIESTGDTGPALQISKNVASTLSTARNVVALSNVFTGAIPGFVLSSKNCYNHIKKCFAPETEYDCGSIEKGLPYNKLYLTKGDHVLGAIKEGCSAVGAGTYVATFGVSRPVLLANKLAHKPFLSSGVKAAFCNSVTYMMTANHAAGVIGGAAALLYENRVYKRASEGLLASKMTESLDSEVYDQVSKGLKESHYQAVKKTILGILEKAFELIADIFDFIALPITASVRLAIKAGLVTVSSGFGLYSVWVNS

SEQ ID NO: 115 - DNA, 423 bp, Chlamydia psittaci
atgagtttggattttttagaagaattttaccgtcgttctatttgtaacaaaggaacggcatttcctgagggcttcatggatattgccgatgtcctatcccattctgcgtctgaacttaaaatcgagtctatgagcgatcttcctgttaacaatttcatcattgcagaatcggcagataaactcactttatttaatgcagattttgctgtttggttagtgcctgagcttgtccaaggagaagctgtgactcgaggatacatcgctttataccattctggaggggattatactccagaaatggcatttcaagcctctggggagtacaatcaatcagcattaattcttgaagcacttcagatatatctacaagacataaaagataccgaaagcacgctacgctctttccgctttaatcaagactag

SEQ ID NO: 116 - protein, 140 aa, Chlamydia psittaci
MSLDFLEEFYRRSICNKGTAFPEGFMDIADVLSHSASELKIESMSDLPVNNFIIAESADKLTLFNADFAVWLVPELVQGEAVTRGYIALYHSGGDYTPEMAFQASGEYNQSALILEALQIYLQDIKDTESTLRSFRFNQD

SEQ ID NO: 117 - DNA, 849 bp, Chlamydia psittaci
atggaattaaataaaacatccgagtctttgtacaattgcaagacagatcgccattcagtacaacaagaagtaggtccagagcctaaagataaccgtgacgttaaagtgttttctttagaaggccgtcaacaatcaaaacaagatcgtcaggataaagtttccagtaaagattctcgtcaagaatctcgaggagctgatgataagcatgtagaagagaaaacatcagcagtatcttctaaagaagaagataaagaagagagtgatggtttcatggcttatgacaatcctacagcaggcatggcatttgtagatgttgctgcttctgtatctagtgaagccgttgtagaaagtactacagtagctatcggcagtgcagatttacagtgggtgcaagatgttattgctagtactgtagaatctatgatggttgctgatgttaacggtcagcaattaatcgagttagttttagatgctgaaggtaatgttcctgacatttttgcaggtgcgaatttaacattagtacaaacaggaacagacctatctgtaaaattctctaattttgtagataatgctcagatggcagaagcaatgagtctcattgtgaataacccttctcagcttgctggtttagtagaggcattaaaaaatcgtcatttgaatttgacagaattggctgttggatcaagtattgtacaattgccaactattgaagaggtgcaaacacctctacatatgattgctgctacaattcatcaaagagatgaagagagagatcaagaaggacaagatcagcagcaacagcaagatcaagaacaaaaccaatataaagttgaagaagcacgtttgtaa

SEQ ID NO: 118 - protein, 282 aa, Chlamydia psittaci
MELNKTSESLYNCKTDRHSVQQEVGPEPKDNRDVKVFSLEGRQQSKQDRQDKVSSKDSRQESRGADDKHVEEKTSAVSSKEEDKEESDGFMAYDNPTAGMAFVDVAASVSSEAVVESTTVAIGSADLQWVQDVIASTVESMMVADVNGQQLIELVLDAEGNVPDIFAGANLTLVQTGTDLSVKFSNFVDNAQMAEAMSLIVNNPSQLAGLVEALKNRHLNLTELAVGSSIVQLPTIEEVQTPLHMIAATIHQRDEERDQEGQDQQQQQDQEQNQYKVEEARL

SEQ ID NO: 119 - DNA, 261 bp, Chlamydia psittaci
atgagtagtggtagcgggagcagttgctcagcatttaattttaatgacatgctcaatggcgtatgtaagtacgtccaaggtgtgcaacaatatttaacagaattagaaacctcaacgcaaggtactgttgacttaggtacgatgtttaatttgcagtttcgtatgcaaattttatcacagtacatggaagcagtatccaacatcttgacagcagtgaacacagagatgattactatggcaagagctgttaaaggaagttaa

SEQ ID NO: 120 - protein, 86 aa, Chlamydia psittaci
MSSGSGSSCSAFNFNDMLNGVCKYVQGVQQYLTELETSTQGTVDLGTMFNLQFRMQILSQYMEAVSNILTAVNTEMITMARAVKGS

SEQ ID NO: 121 - DNA, 729 bp, Chlamydia psittaci
atggaaccttatttagatttattagataaaaacatcaaagaaaaacatatgttagatcatcccttttatatgaagtggtctaagggagaattaacgaaagagcagttaaaagaatacgctaaggattactacctccacatcaaagcttttcctcgttatctttcagcagttcatagccgctgtgataatttagaagcgcgtaaattgcttcttgataaccttatggatgaagaaacaggccatcctaatcacatagatctatggaaaaattttgcctatgcgttaggtgttactgaggaagaattagaaaatcatgttcctagtgcagcagcacaaaagaaagtggatacatttttacgttggtgtactggagattccttatctgctggtgtagctgctttatatacctatgaaagccaaatccctacagttgcagagactaaaatctcgggattaaaacagtatttcggctttacggctcctgaagattatgagtacttcacagtacatcaagatgttgatgtaagacattctcgtgaagaaaaagaattaatagagtcgttgctaaataatgatagcgataaggttctacaagcttcaaaagaagtttgtgatgctttatacggctttttagatactttcttagatgaaaaagacgcctgctcagcaacttcatcttctgttgcggattctaaaccatcttcatgttgttgtcgttgccgtcattaa

SEQ ID NO: 122 - protein, 242 aa, Chlamydia psittaci
MEPYLDLLDKNIKEKHMLDHPFYMKWSKGELTKEQLKEYAKDYYLHIKAFPRYLSAVHSRCDNLEARKLLLDNLMDEETGHPNHIDLWKNFAYALGVTEEELENHVPSAAAQKKVDTFLRWCTGDSLSAGVAALYTYESQIPTVAETKISGLKQYFGFTAPEDYEYFTVHQDVDVRHSREEKELIESLLNNDSDKVLQASKEVCDALYGFLDTFLDEKDACSATSSSVADSKPSSCCCRCRH

SEQ ID NO: 123 - DNA, 234 bp, Chlamydia psittaci
atgaatgaaggtattcaaaccgtttcttttaacaagacacaccgactcaccgcgaaatctacagttagtttagaaatgcccgtagcgacacagaaacttcaaggtaaagaaggcatgcccgcagcggcaagtttagaagctgatttcttaagagcagaagctatacttgcagaaatgcgtgaaattcgcggctgtttagaacattcattggaaacgctagttcctagagactag

SEQ ID NO: 124 - protein, 77 aa, Chlamydia psittaci
MNEGIQTVSFNKTHRLTAKSTVSLEMPVATQKLQGKEGMPAAASLEADFLRAEAILAEMREIRGCLEHSLETLVPRD

SEQ ID NO: 125 - DNA, 231 bp, Chlamydia psittaci
atgaaaaataaactacatgatttgttgaatcagctttatgaaaatcaaaaatctcgtttacaaagcatgggggagcagatcgtcccgaatttaacttctgatgatgtattacagcccatggatttttctatgctggaagaaaatcctttctttcggtttgaggaaggtgttctttctggtcttggagaggctcgggcggcgattttggcattatttgccgaagaaggttaa

SEQ ID NO: 126 - protein, 76 aa, Chlamydia psittaci
MKNKLHDLLNQLYENQKSRLQSMGEQIVPNLTSDDVLQPMDFSMLEENPFFRFEEGFFLVLERLGRRFWHYLPKKV

SEQ ID NO: 127 - DNA, 519 bp, Chlamydia psittaci
atgtggttctcttctccagcacttcaaactagccctagagccgctattgacgttcccgggacatctatcacaggtggaccaaatacagcaacagccgatgaaattattgcaaaatttgcgaaagattccaatcctctgattatcaccgtgtactatgtataccagtctgtattggtagcgcaggacaacttatctattatcgctcaagaacttcagtctaatgcttctgcccagacttttttaaacaaccaagaagctttataccaatacgtaaccatacctaagaacaaattaaacgataactcgtcggcgttcttacaagatgttcaatcaacaaaccaagctgttggagcttctcgacaagcactacaaaaccaaatttcaggtttaggaaatggtgctcaggttatctcaagtaacttgaacacgaacaataacattatccaacagtctctacaagtaggccaggcgttaattcaaacgttctcacaaattgtaagtttgatagcaaacatttaa

SEQ ID NO: 128 - protein, 172 aa, Chlamydia psittaci
MWFSSPALQTSPRAAIDVPGTSITGGPNTATADEIIAKFAKDSNPLIITVYYVYQSVLVAQDNLSIIAQELQSNASAQTFLNNQEALYQYVTIPKNKLNDNSSAFLQDVQSTNQAVGASRQALQNQISGLGNGAQVISSNLNTNNNIIQQSLQVGQALIQTFSQIVSLIANI

SEQ ID NO: 129 - DNA, 1443 bp, Chlamydia psittaci
atgaacactcccctgccttcagcagttccctctaccaacatgacgttaaaggaagatgcctcctcttcatcctcagcatcaacatcttccagtattttaaagactgcagcaggagatgttgcgctttccgtattcacttccgaaggaaccacaccagcttccttaaactctctagttgctcttgcccttgcgcaaatttctgcagcttctggagaaaatgccaatcctttacaagattgtgctcataatcttgtcttcctttctccagaaactattgaagttgaaatactcatctctgatcttcttcaaactttagagactacagatcttataaacacacaggaagagtcctcaagtttaggaaaacaagaacagcgccttcctcaagaaggatgcaaaccacaggatttagcaccaagatctacaatagattcgtacggcacaccgaaagcattacaacaacctgccgtgaaactcgctgtccgctattcttctgcaaaggctcctgattctcggccttatacaagttcctcctcaccgcagcatacttcgggacaatttgcccaacgtgctgcccaggcgccaggaatactgcaacactcccaaacaaaaaaagatggagagcaaatttcttctcaatctcacaattcctttattgcggagaaaaaagaacagcagattttcaccacaaagtctcaagaatctcagcaggatcgtgaaaaccgagatcaaaaacaagatcgccaacatgatggccaacatcaagatgatgatgatgatcagcaaaaaggtagggggaaaaaatataaatcaaagacttctgccgaagcagttcctgctgatctctccgtagcgcatctacgctatcttaatgaggtacgccaatctcgagaaattcatgttgaggaagaaaaaacatttaagaagaaagcgcaatctccgatggcacttttttctgctccaactcctccagcaggatttaccccaattcctactccaaagattgaaaacgtattcatacgttttatgaaacttatggaaaggattctgggacaggccgaagccgaagcccaagaattatatcttcgcgttaaagagcgtaccgataatgtagacacattaatgctgctcatttctaaaattaactctgaaaaaggtgctattgactggcgagatgattcagaaatgaaagctctcgtggatcaagcaaaaaaactgggtgtaacgataacagatactctgcaatggtctgaagaagagaaaaaacttctaaaagaaaatattcagatgcgtaaagaaaacctggaaaaaatcacccagcttgaacgaacagacatgcaaaggcatcttcaagaagtctcacaatgtcaccaagcacgctccaacgccttgaaacttctcaaagaactcatggatacctttatttacaatttgcgtccgtaa

SEQ ID NO: 130 - protein, 480 aa, Chlamydia psittaci
MNTPLPSAVPSTNMTLKEDASSSSSASTSSSILKTAAGDVALSVFTSEGTTPASLNSLVALALAQISAASGENANPLQDCAHNLVFLSPETIEVEILISDLLQTLETTDLINTQEESSSLGKQEQRLPQEGCKPQDLAPRSTIDSYGTPKALQQPAVKLAVRYSSAKAPDSRPYTSSSSPQHTSGQFAQRAAQAPGILQHSQTKKDGEQISSQSHNSFIAEKKEQQIFTTKSQESQQDRENRDQKQDRQHDGQHQDDDDDQQKGRGKKYKSKTSAEAVPADLSVAHLRYLNEVRQSREIHVEEEKTFKKKAQSPMALFSAPTPPAGFTPIPTPKIENVFIRFMKLMERILGQAEAEAQELYLRVKERTDNVDTLMLLISKINSEKGAIDWRDDSEMKALVDQAKKLGVTITDTLQWSEEEKKLLKENIQMRKENLEKITQLERTDMQRHLQEVSQCHQARSNALKLLKELMDTFIYNLRP

SEQ ID NO: 131 - DNA, 1083 bp, Chlamydia psittaci
atgaagaacaaaacactaagcgttttctctttggtggggttggtttgttcggtaggagttttaccaggcaatgaaagttctctccctgtacaacgggagtatccaagtcgtacagaaagaatccctgaagatcctgccggaattgctattcatgatcgtgtgttatttaaaatcgatgaagataacgttgttacgactttagatgttatacagaagcttaaccttctttttgcttcttcataccctcagcttatggattcttatcctgcgcgttcgcaatactatacagccatgtggccagtagttttagaatctgtaatagacgagtttcttatggttgccgatgctaagactaaaaaaatccaggtggactctactacagtgaatgaagaaattgaagcaatgtttggtagagatctatctcctttgtatgtacattttgacatgacccccgaagatgttttcaacgtggtaaatcgtaccctaattgctcagagagtcatgggtatgatggtgcgctctaaggtaatgttgaaagttaccccagggaaaattcgcgaacattataatcagctggctgaagatgcagcaaacactactgtatggaaatacagagttgttacgattaaagcagctacagagtcattatcgagccaaattgccgataaagtctgcgctaggttaaatgaaacgcaaagctggaataaagagcgtttatctgctcttactctttctcaaggagggcagtttgtctgttctgaagagtttacacgcaatgataaagaattatcagaagctcataaaatggaattgtcctcggtaaactacccacaaactatttgcagcctacctaaagcccataagtcaggacataagctttatgtgcttcttgataaatctgcaatggcaatgcagcccctagaagaaatggaaacgcagataaagcagacgctatttatgaattatgctgggactatagaaagtcagtataaaatgaaattgcgtacacgctatggatttgactcttcaacgattgctaaattgctttctgaagaagctccgcctctattttcattgctttag

SEQ ID NO: 132 - protein, 360 aa, Chlamydia psittaci
MKNKTLSVFSLVGLVCSVGVLPGNESSLPVQREYPSRTERIPEDPAGIAIHDRVLFKIDEDNVVTTLDVIQKLNLLFASSYPQLMDSYPARSQYYTAMWPVVLESVIDEFLMVADAKTKKIQVDSTTVNEEIEAMFGRDLSPLYVHFDMTPEDVFNVVNRTLIAQRVMGMMVRSKVMLKVTPGKIREHYNQLAEDAANTTVWKYRVVTIKAATESLSSQIADKVCARLNETQSWNKERLSALTLSQGGQFVCSEEFTRNDKELSEAHKMELSSVNYPQTICSLPKAHKSGHKLYVLLDKSAMAMQPLEEMETQIKQTLFMNYAGTIESQYKMKLRTRYGFDSSTIAKLLSEEAPPLFSLL

SEQ ID NO: 133 - DNA, 34 bp, Artificial Sequence
  Synthetic DNA
agtcaagcttatgattgtacggacatagagaccg

SEQ ID NO: 134 - DNA, 35 bp, Artificial Sequence
  Synthetic DNA
agtcaagcttgtaggtttattaaaggggatgtacc

SEQ ID NO: 135 - DNA, 34 bp, Artificial Sequence
  Synthetic DNA
agtcaagcttgtagctgttcttttcagagagctt

SEQ ID NO: 136 - DNA, 33 bp, Artificial Sequence
  Synthetic DNA
agtcaagctttactttttgaaggctagtacgtt

SEQ ID NO: 137 - DNA, 33 bp, Artificial Sequence
  Synthetic DNA
agtcaagcttggaaggaatatcgtttacctgct

SEQ ID NO: 138 - DNA, 33 bp, Artificial Sequence
  Synthetic DNA
agtcaagcttgtagatgcccatcctaccaacag

SEQ ID NO: 139 - DNA, 34 bp, Artificial Sequence
  Synthetic DNA
agtcaagctttccctaaaatgaataaactaagga

SEQ ID NO: 140 - DNA, 33 bp, Artificial Sequence
  Synthetic DNA
agtcaagcttaagtgtcattgaaaaggttcagg

SEQ ID NO: 141 - DNA, 32 bp, Artificial Sequence
  Synthetic DNA
agtcaagcttgccaaccaacggttagacgaaa

SEQ ID NO: 142 - DNA, 34 bp, Artificial Sequence
  Synthetic DNA
agtcaagcttgcgtgccttttagaaaatttagca

SEQ ID NO: 143 - DNA, 35 bp, Artificial Sequence
  Synthetic DNA
agtcaagcttgtaacgactccttctttcaacgatt

SEQ ID NO: 144 - DNA, 33 bp, Artificial Sequence
  Synthetic DNA
agtcaagcttccattaaggatctaaaacaattt

SEQ ID NO: 145 - DNA, 33 bp, Artificial Sequence
  Synthetic DNA
agtcaagctttgtttgagatgaattcgcatttt

SEQ ID NO: 146 - DNA, 34 bp, Artificial Sequence
  Synthetic DNA
agtcaagcttttgcatgaattcgtaaaatagaaa

SEQ ID NO: 147 - DNA, 35 bp, Artificial Sequence
  Synthetic DNA
agtcaagcttgtagggtttgtggagaaaattgtta

SEQ ID NO: 148 - DNA, 33 bp, Artificial Sequence
  Synthetic DNA
agtcaagcttcttctattagcctgtttccaatt

SEQ ID NO: 149 - DNA, 34 bp, Artificial Sequence
  Synthetic DNA
agtcaagcttctgttgttgaattaatagcttctt

SEQ ID NO: 150 - DNA, 35 bp, Artificial Sequence
  Synthetic DNA
agtcaagcttgtgagaaaaacaacaattcttatcc

SEQ ID NO: 151 - DNA, 34 bp, Artificial Sequence
  Synthetic DNA
agtcaagcttacgactcaattaacagtgatgcaa

SEQ ID NO: 152 - DNA, 34 bp, Artificial Sequence
  Synthetic DNA
agtcaagctttcaagacaattagagagaaagagc

SEQ ID NO: 153 - DNA, 35 bp, Artificial Sequence
  Synthetic DNA
agtcaagcttgtaggcattgcttaaaaataaaatg

SEQ ID NO: 154 - DNA, 35 bp, Artificial Sequence
  Synthetic DNA
agtcaagcttgtaatcggattttaaaccaactttt

SEQ ID NO: 155 - DNA, 35 bp, Artificial Sequence
  Synthetic DNA
agtcaagcttgtaggaatttttattacgagtttca

SEQ ID NO: 156 - DNA, 33 bp, Artificial Sequence
  Synthetic DNA
agtcaagcttcaagaagcgttaagaaaacgaaa

SEQ ID NO: 157 - DNA, 34 bp, Artificial Sequence
  Synthetic DNA
agtcaagcttttattaaaatttgttaaagggagg

SEQ ID NO: 158 - DNA, 35 bp, Artificial Sequence
  Synthetic DNA
agtcaagcttgtaaactaattcggattaaaaatga

SEQ ID NO: 159 - DNA, 34 bp, Artificial Sequence
  Synthetic DNA
agtcaagcttgaaggaaaagaagttctcataaag

SEQ ID NO: 160 - DNA, 32 bp, Artificial Sequence
  Synthetic DNA
agtcaagcttgtagcgtctggaaaaattctgg

SEQ ID NO: 161 - DNA, 33 bp, Artificial Sequence
  Synthetic DNA
agtcaagcttgggtaagaacaccttctaatatg

SEQ ID NO: 162 - DNA, 32 bp, Artificial Sequence
  Synthetic DNA
gactaagcttgtaatctatttttagataggaa

SEQ ID NO: 163 - DNA, 33 bp, Artificial Sequence
  Synthetic DNA
agtcaagcttcccgtaattactgtccgtacaac

SEQ ID NO: 164 - DNA, 31 bp, Artificial Sequence
  Synthetic DNA
gactaagcttattatatagacagattaaaat

SEQ ID NO: 165 - DNA, 32 bp, Artificial Sequence
  Synthetic DNA
agtcaagcttagggttttaagtcggtttgatg

SEQ ID NO: 166 - DNA, 34 bp, Artificial Sequence
  Synthetic DNA
agtcaagctttcgataatcataattcaaagcgta

SEQ ID NO: 167 - DNA, 34 bp, Artificial Sequence
  Synthetic DNA
agtcaagctttccatgaattctaaaatgattagg

SEQ ID NO: 168 - DNA, 32 bp, Artificial Sequence
  Synthetic DNA
agtcaagcttaatttaaaggaactcaggtgaa

SEQ ID NO: 169 - DNA, 33 bp, Artificial Sequence
  Synthetic DNA
agtcaagcttgtaacatggggtatggaaagacc

SEQ ID NO: 170 - DNA, 33 bp, Artificial Sequence
  Synthetic DNA
agtcaagcttgtaagcacctgctctcgaataca

SEQ ID NO: 171 - DNA, 34 bp, Artificial Sequence
  Synthetic DNA
agtcaagcttgtaacggtctctttgcttgttttt

SEQ ID NO: 172 - DNA, 34 bp, Artificial Sequence
  Synthetic DNA
agtcaagcttgtaacgatccgaacttatcgtagt

SEQ ID NO: 173 - DNA, 34 bp, Artificial Sequence
  Synthetic DNA
agtcaagcttgtaaattcatgggcctaatgataa

SEQ ID NO: 174 - DNA, 34 bp, Artificial Sequence
  Synthetic DNA
agtcaagcttgtaattggggaatctctgggagac

SEQ ID NO: 175 - DNA, 32 bp, Artificial Sequence
  Synthetic DNA
agtcaagcttgtaatggaggattggctaagga

SEQ ID NO: 176 - DNA, 35 bp, Artificial Sequence
  Synthetic DNA
agtcaagcttgtaagagggaccttacctattgctt

SEQ ID NO: 177 - DNA, 31 bp, Artificial Sequence
  Synthetic DNA
agtcaagcttgcggaatggttaaaggaaacg

SEQ ID NO: 178 - DNA, 33 bp, Artificial Sequence
  Synthetic DNA
agtcaagcttgtaaaaccagcctagccttcaaa

SEQ ID NO: 179 - DNA, 34 bp, Artificial Sequence
  Synthetic DNA
agtcaagcttgtaacctgcttctttaggaactac

SEQ ID NO: 180 - DNA, 34 bp, Artificial Sequence
  Synthetic DNA
agtcaagcttgtaaaattcagcacaggcttgtaa

SEQ ID NO: 181 - DNA, 32 bp, Artificial Sequence
  Synthetic DNA
gactaagcttgtaaattggagattgtagtagc

SEQ ID NO: 182 - DNA, 32 bp, Artificial Sequence
  Synthetic DNA
atgcaagcttacgagcaagagcataaaatcca

SEQ ID NO: 183 - DNA, 33 bp, Artificial Sequence
  Synthetic DNA
agtcaagcttgaattgctaacagacactaaagg

SEQ ID NO: 184 - DNA, 35 bp, Artificial Sequence
  Synthetic DNA
agtcaagcttgttttaaaaagccttgtaaggaggt

SEQ ID NO: 185 - DNA, 36 bp, Artificial Sequence
  Synthetic DNA
agtcaagcttgtaagaattaccataaatcagaggaa

SEQ ID NO: 186 - DNA, 36 bp, Artificial Sequence
  Synthetic DNA
agtcaagcttgtaacagaaggaaaagcataccactg

SEQ ID NO: 187 - DNA, 33 bp, Artificial Sequence
  Synthetic DNA
agtcaagcttgccataagtcgtttacaagatcg

SEQ ID NO: 188 - DNA, 31 bp, Artificial Sequence
  Synthetic DNA
agtcaagcttgtgaatcagggggaagctagt

SEQ ID NO: 189 - DNA, 32 bp, Artificial Sequence
  Synthetic DNA
agtcaagcttcgcaagatgatataaaggtcca

SEQ ID NO: 190 - DNA, 34 bp, Artificial Sequence
  Synthetic DNA
agtcaagctttaaataaccaagtattggggttta

SEQ ID NO: 191 - DNA, 34 bp, Artificial Sequence
  Synthetic DNA
agtcaagcttgtagacatggctgtcagatcttgg

SEQ ID NO: 192 - DNA, 33 bp, Artificial Sequence
  Synthetic DNA
agtcaagcttctctttttaaagacaacgcgaat

SEQ ID NO: 193 - DNA, 35 bp, Artificial Sequence
  Synthetic DNA
agtcaagcttgtaattaatcttgcggagatgttgg

SEQ ID NO: 194 - DNA, 33 bp, Artificial Sequence
  Synthetic DNA
agtcaagcttgtaagcctgcttggtcttctgtt

SEQ ID NO: 195 - DNA, 34 bp, Artificial Sequence
  Synthetic DNA
atgcaagcttgtaagggaagatgtccgttcagtt

SEQ ID NO: 196 - DNA, 34 bp, Artificial Sequence
  Synthetic DNA
agtcaagcttatggctatgaagagcaatttattc

SEQ ID NO: 197 - DNA, 30 bp, Artificial Sequence
  Synthetic DNA
agtcaagcttgtaactcccgcaccaacctc

SEQ ID NO: 198 - DNA, 35 bp, Artificial Sequence
  Synthetic DNA
agtcaagcttgtaatctggttaacacatgctgagg

SEQ ID NO: 199 - DNA, 35 bp, Artificial Sequence
  Synthetic DNA
agtcaagcttgtaagagccgagcatagatagaaac

SEQ ID NO: 200 - DNA, 34 bp, Artificial Sequence
  Synthetic DNA
agtcaagcttttaataagctgaataaaccaatga

SEQ ID NO: 201 - DNA, 34 bp, Artificial Sequence
  Synthetic DNA
agtcaagcttgtaacatgacgacatcaccttatt

SEQ ID NO: 202 - DNA, 34 bp, Artificial Sequence
  Synthetic DNA
agtcaagcttgtaagtcaggaagtccgtggtgat

SEQ ID NO: 203 - DNA, 32 bp, Artificial Sequence
  Synthetic DNA
agtcaagcttgtaatttgcggttgggaaataa

SEQ ID NO: 204 - DNA, 34 bp, Artificial Sequence
  Synthetic DNA
agtcaagcttgtaacccttgcctctattttgaga

SEQ ID NO: 205 - DNA, 37 bp, Artificial Sequence
  Synthetic DNA
agtcaagcttgtaattgcgagaaggattaaatcttag

SEQ ID NO: 206 - DNA, 32 bp, Artificial Sequence
  Synthetic DNA
agtcaagcttatctcaaacatcaagtgctgaa

SEQ ID NO: 207 - DNA, 31 bp, Artificial Sequence
  Synthetic DNA
agtcaagcttgtaagcctcgtcaaatcctga

SEQ ID NO: 208 - DNA, 31 bp, Artificial Sequence
  Synthetic DNA
agtcaagctttcttcaggaactttaaaaaca

SEQ ID NO: 209 - DNA, 33 bp, Artificial Sequence
  Synthetic DNA
agtcaagctttaatagagctcattggaggagga

SEQ ID NO: 210 - DNA, 36 bp, Artificial Sequence
  Synthetic DNA
agtcaagcttgtaatgtagaaaagcgcagagagaaa

SEQ ID NO: 211 - DNA, 35 bp, Artificial Sequence
  Synthetic DNA
agtcaagcttgtaatcgaaaaccagttaggttgaa

SEQ ID NO: 212 - DNA, 34 bp, Artificial Sequence
  Synthetic DNA
agtcaagcttgtaatgggtcctaatggaggcttt

SEQ ID NO: 213 - DNA, 32 bp, Artificial Sequence
  Synthetic DNA
agtcaagcttgacctccttgtaaccattctcg

SEQ ID NO: 214 - DNA, 33 bp, Artificial Sequence
  Synthetic DNA
agtcaagctttccttaagaggataaatccacag

SEQ ID NO: 215 - DNA, 33 bp, Artificial Sequence
  Synthetic DNA
agtcaagcttttcgaaaacataaataatgtgga

SEQ ID NO: 216 - DNA, 33 bp, Artificial Sequence
  Synthetic DNA
agtcaagcttgtgcaggaaaatgatgttttagc

SEQ ID NO: 217 - DNA, 30 bp, Artificial Sequence
  Synthetic DNA
agtcaagcttcagcctggtttcgtaatttt

SEQ ID NO: 218 - DNA, 36 bp, Artificial Sequence
  Synthetic DNA
agtcaagcttgtaaaccacaccaatattatttagga

SEQ ID NO: 219 - DNA, 34 bp, Artificial Sequence
  Synthetic DNA
agtcaagcttgtaacctgggcaggtcaaaatcta

SEQ ID NO: 220 - DNA, 34 bp, Artificial Sequence
  Synthetic DNA
agtcaagcttgtaatgggtttccggtttcaatca

SEQ ID NO: 221 - DNA, 35 bp, Artificial Sequence
  Synthetic DNA
agtcaagcttgtaagccaagagcaggataaaacga

SEQ ID NO: 222 - DNA, 35 bp, Artificial Sequence
  Synthetic DNA
agtcaagcttgtaaatttttcctataagctggttc

SEQ ID NO: 223 - DNA, 35 bp, Artificial Sequence
  Synthetic DNA
agtcaagcttttgataagaaattagccaacatagg

SEQ ID NO: 224 - DNA, 33 bp, Artificial Sequence
  Synthetic DNA
agtcaagcttttcggtttaagtaatagaagtgg

SEQ ID NO: 225 - DNA, 35 bp, Artificial Sequence
  Synthetic DNA
agtcaagcttgtaagacggtgccttcaataacaaa

SEQ ID NO: 226 - DNA, 33 bp, Artificial Sequence
  Synthetic DNA
agtcaagcttgtaaaaacagggaaagacgatga

SEQ ID NO: 227 - DNA, 32 bp, Artificial Sequence
  Synthetic DNA
agtcaagcttgtaagcggttacgtggagtcaa

SEQ ID NO: 228 - DNA, 33 bp, Artificial Sequence
  Synthetic DNA
agtcaagcttgtaatccggagcctttctcctat

SEQ ID NO: 229 - DNA, 34 bp, Artificial Sequence
  Synthetic DNA
agtcaagcttgtaaagatgaatgattccagaaag

SEQ ID NO: 230 - DNA, 33 bp, Artificial Sequence
  Synthetic DNA
agtcaagcttggactaagtaaaacggagcagga

SEQ ID NO: 231 - DNA, 34 bp, Artificial Sequence
  Synthetic DNA
agtcaagcttttcgaggtttatttaattcttcca

SEQ ID NO: 232 - DNA, 32 bp, Artificial Sequence
  Synthetic DNA
agtcaagcttacgtatcaaccgtaaaatggtg

SEQ ID NO: 233 - DNA, 32 bp, Artificial Sequence
  Synthetic DNA
agtcaagcttgtaagcaaaaacaacgggaatc

SEQ ID NO: 234 - DNA, 36 bp, Artificial Sequence
  Synthetic DNA
agtcaagcttgtaaatgaagagtgctgtgttaaagt

SEQ ID NO: 235 - DNA, 34 bp, Artificial Sequence
  Synthetic DNA
agtcaagcttgtaacacaatcttgggcaagagac

SEQ ID NO: 236 - DNA, 36 bp, Artificial Sequence
  Synthetic DNA
agtcaagcttgtaagagaagagaaaggctaaggatg

SEQ ID NO: 237 - DNA, 31 bp, Artificial Sequence
  Synthetic DNA
agtcaagcttataaacccctgtcttaaccca

SEQ ID NO: 238 - DNA, 30 bp, Artificial Sequence
  Synthetic DNA
agtcaagctttcctttctaaaaggcctgga

SEQ ID NO: 239 - DNA, 35 bp, Artificial Sequence
  Synthetic DNA
agtcaagcttgcaaatctaaggagttaggttaggg

SEQ ID NO: 240 - DNA, 32 bp, Artificial Sequence
  Synthetic DNA
agtcaagcttgtaatacaggatggtgcccact

SEQ ID NO: 241 - DNA, 29 bp, Artificial Sequence
  Synthetic DNA
agtcaagcttgcgcaaataaacgatacaa

SEQ ID NO: 242 - DNA, 33 bp, Artificial Sequence
  Synthetic DNA
agtcaagcttcattgtgttataattgcaggcta

SEQ ID NO: 243 - DNA, 31 bp, Artificial Sequence
  Synthetic DNA
agtcaagctttgacgccccttaaaataaaga

SEQ ID NO: 244 - DNA, 30 bp, Artificial Sequence
  Synthetic DNA
agtcaagcttccgcaaaatggtttaacaga

SEQ ID NO: 245 - DNA, 36 bp, Artificial Sequence
  Synthetic DNA
agtcaagcttgtaagcttgggaatctacacattttt

SEQ ID NO: 246 - DNA, 33 bp, Artificial Sequence
  Synthetic DNA
agtcaagcttgtaaaagctgaataagcccatga

SEQ ID NO: 247 - DNA, 33 bp, Artificial Sequence
  Synthetic DNA
agtcaagcttgtaaccgtgaggagtctgaacaa

SEQ ID NO: 248 - DNA, 35 bp, Artificial Sequence
  Synthetic DNA
agtcaagcttttaatcgaagaaaataggtaggaaa

SEQ ID NO: 249 - DNA, 35 bp, Artificial Sequence
  Synthetic DNA
agtcaagcttgtaacgcttctacatccccataatt

SEQ ID NO: 250 - DNA, 29 bp, Artificial Sequence
  Synthetic DNA
agtctctagatgcttttcggaccatagtc

SEQ ID NO: 251 - DNA, 31 bp, Artificial Sequence
  Synthetic DNA
agtctctagaatcctcttcccaaggaatcag

SEQ ID NO: 252 - DNA, 30 bp, Artificial Sequence
  Synthetic DNA
agtctctagatcgaatcgatttggaaggag

SEQ ID NO: 253 - DNA, 33 bp, Artificial Sequence
  Synthetic DNA
agtctctagattcaataatgccagagctttttc

SEQ ID NO: 254 - DNA, 30 bp, Artificial Sequence
  Synthetic DNA
agtctctagattcatccacccaaatagcac

SEQ ID NO: 255 - DNA, 29 bp, Artificial Sequence
  Synthetic DNA
agtctctagaagtaagagggagcccagga

SEQ ID NO: 256 - DNA, 30 bp, Artificial Sequence
  Synthetic DNA
agtctctagatgacgggggaggtgtattag

SEQ ID NO: 257 - DNA, 31 bp, Artificial Sequence
  Synthetic DNA
agtctctagataggaggcctttcgattgttt

SEQ ID NO: 258 - DNA, 29 bp, Artificial Sequence
  Synthetic DNA
agcttctagatggaaaggatacggattgg

SEQ ID NO: 259 - DNA, 30 bp, Artificial Sequence
  Synthetic DNA
agtctctagagaatgggggaatacaaatca

SEQ ID NO: 260 - DNA, 30 bp, Artificial Sequence
  Synthetic DNA
agtctctagaggcgagagaaagtttcgtta

SEQ ID NO: 261 - DNA, 29 bp, Artificial Sequence
  Synthetic DNA
agtctctagacaacttgttcatgcgacag

SEQ ID NO: 262 - DNA, 30 bp, Artificial Sequence
  Synthetic DNA
agcttctagacgcatccgaaagacttttct

SEQ ID NO: 263 - DNA, 32 bp, Artificial Sequence
  Synthetic DNA
agtctctagaacgttctaagccgtaattctcg

SEQ ID NO: 264 - DNA, 30 bp, Artificial Sequence
  Synthetic DNA
agtctctagatgcggggatcgtataagcta

SEQ ID NO: 265 - DNA, 31 bp, Artificial Sequence
  Synthetic DNA
agtctctagaaggtgattgcttcccaagtct

SEQ ID NO: 266 - DNA, 31 bp, Artificial Sequence
  Synthetic DNA
agtctctagaataagagtcgagggcttgcac

SEQ ID NO: 267 - DNA, 32 bp, Artificial Sequence
  Synthetic DNA
agtctctagatgtaggaatacctggagtcgtg

SEQ ID NO: 268 - DNA, 31 bp, Artificial Sequence
  Synthetic DNA
agtctctagagcgctgagtagcgtcgtgtaa

SEQ ID NO: 269 - DNA, 31 bp, Artificial Sequence
  Synthetic DNA
agcttctagagaattcaagatgatccaaaca

SEQ ID NO: 270 - DNA, 30 bp, Artificial Sequence
  Synthetic DNA
agtctctagaagtcttatgaagcaaagcag

SEQ ID NO: 271 - DNA, 33 bp, Artificial Sequence
  Synthetic DNA
agtctctagaatttatatgccacgccttctttc

SEQ ID NO: 272 - DNA, 29 bp, Artificial Sequence
  Synthetic DNA
agtctctagaggaggtttccgagacgatt

SEQ ID NO: 273 - DNA, 30 bp, Artificial Sequence
  Synthetic DNA
agtctctagaatcccagtcgtgagaagcat

SEQ ID NO: 274 - DNA, 34 bp, Artificial Sequence
  Synthetic DNA
agtctctagagagaagaatatctgcaactagagc

SEQ ID NO: 275 - DNA, 30 bp, Artificial Sequence
  Synthetic DNA
agtctctagaggtaacagtgcatgcgaaag

SEQ ID NO: 276 - DNA, 32 bp, Artificial Sequence
  Synthetic DNA
agtctctagacaaaataggaatagcagcttgg

SEQ ID NO: 277 - DNA, 33 bp, Artificial Sequence
  Synthetic DNA
agtctctagacggtcttaaactacttttcaatg

SEQ ID NO: 278 - DNA, 32 bp, Artificial Sequence
  Synthetic DNA
agtctctagaatcaaccacatcttctggacaa

SEQ ID NO: 279 - DNA, 31 bp, Artificial Sequence
  Synthetic DNA
gacttctagatccaggtttttcggaagcaga

SEQ ID NO: 280 - DNA, 28 bp, Artificial Sequence
  Synthetic DNA
agtctctagaagtagacgggagttgctg

SEQ ID NO: 281 - DNA, 31 bp, Artificial Sequence
  Synthetic DNA
gacttctagacttaaaaaatacccaggaaca

SEQ ID NO: 282 - DNA, 30 bp, Artificial Sequence
  Synthetic DNA
agtctctagaccgaatcgcttcattcgtag

SEQ ID NO: 283 - DNA, 32 bp, Artificial Sequence
  Synthetic DNA
agtctctagaaaggatcagatgtgctttaggg

SEQ ID NO: 284 - DNA, 30 bp, Artificial Sequence
  Synthetic DNA
agtctctagacgaaaattgggtagcctgac

SEQ ID NO: 285 - DNA, 30 bp, Artificial Sequence
  Synthetic DNA
agtctctagaatcaaagttgctgcaggatt

SEQ ID NO: 286 - DNA, 30 bp, Artificial Sequence
  Synthetic DNA
agtctctagactcggcatccttttgttttg

SEQ ID NO: 287 - DNA, 30 bp, Artificial Sequence
  Synthetic DNA
agtctctagattcttcttggtgcctgctaa

SEQ ID NO: 288 - DNA, 30 bp, Artificial Sequence
  Synthetic DNA
agtctctagaagtgcagctggatagggttg

SEQ ID NO: 289 - DNA, 30 bp, Artificial Sequence
  Synthetic DNA
agtctctagatccggatgcggtagtagttg

SEQ ID NO: 290 - DNA, 31 bp, Artificial Sequence
  Synthetic DNA
agtctctagataaccctgtggatgacgtttt

SEQ ID NO: 291 - DNA, 30 bp, Artificial Sequence
  Synthetic DNA
agtctctagagggactaacgacctcagcac

SEQ ID NO: 292 - DNA, 30 bp, Artificial Sequence
  Synthetic DNA
agtctctagaaacaccaccgacatcacaaa

SEQ ID NO: 293 - DNA, 30 bp, Artificial Sequence
  Synthetic DNA
agtctctagaaatgaggacctctccttcgt

SEQ ID NO: 294 - DNA, 31 bp, Artificial Sequence
  Synthetic DNA
agtctctagattgtccatcaaagcctacaat

SEQ ID NO: 295 - DNA, 29 bp, Artificial Sequence
  Synthetic DNA
agtctctagagctttttgcataggggaag

SEQ ID NO: 296 - DNA, 31 bp, Artificial Sequence
  Synthetic DNA
agtctctagattgtagaccagcggtgacact

SEQ ID NO: 297 - DNA, 30 bp, Artificial Sequence
  Synthetic DNA
agtctctagagcgttctggagtgacgaaac

SEQ ID NO: 298 - DNA, 31 bp, Artificial Sequence
  Synthetic DNA
gacttctagaaacaattgtatgattccatcc

SEQ ID NO: 299 - DNA, 30 bp, Artificial Sequence
  Synthetic DNA
atgctctagacctttggcggactttttctt

SEQ ID NO: 300 - DNA, 30 bp, Artificial Sequence
  Synthetic DNA
agtctctagatacccattcttgaccggaaa

SEQ ID NO: 301 - DNA, 30 bp, Artificial Sequence
  Synthetic DNA
agtctctagactgagcaaaggagctgacag

SEQ ID NO: 302 - DNA, 32 bp, Artificial Sequence
  Synthetic DNA
agtctctagaagcatcagtgcaatgaggataa

SEQ ID NO: 303 - DNA, 29 bp, Artificial Sequence
  Synthetic DNA
agtctctagatccaaaacgatcctgttcc

SEQ ID NO: 304 - DNA, 30 bp, Artificial Sequence
  Synthetic DNA
agtctctagattccacacaagagaccacca

SEQ ID NO: 305 - DNA, 30 bp, Artificial Sequence
  Synthetic DNA
agtctctagatcgtgtttgctttccttcca

SEQ ID NO: 306 - DNA, 30 bp, Artificial Sequence
  Synthetic DNA
agtctctagagacagtgccttgtgttgatg

SEQ ID NO: 307 - DNA, 30 bp, Artificial Sequence
  Synthetic DNA
agtctctagatcgaagcagcgcatgtaact

SEQ ID NO: 308 - DNA, 31 bp, Artificial Sequence
  Synthetic DNA
agtctctagaagagtcgcgtcctatagacca

SEQ ID NO: 309 - DNA, 30 bp, Artificial Sequence
  Synthetic DNA
agcttctagacgtgtgagtcccctgaactt

SEQ ID NO: 310 - DNA, 32 bp, Artificial Sequence
  Synthetic DNA
agtctctagactctgtagtcagctggtcgttg

SEQ ID NO: 311 - DNA, 31 bp, Artificial Sequence
  Synthetic DNA
agtctctagacgagagaggaagttcagcgta

SEQ ID NO: 312 - DNA, 31 bp, Artificial Sequence
  Synthetic DNA
atgctctagaagctcccttaacgacttctgg

SEQ ID NO: 313 - DNA, 32 bp, Artificial Sequence
  Synthetic DNA
agtctctagaagggtaacacactaggggaaga

SEQ ID NO: 314 - DNA, 31 bp, Artificial Sequence
  Synthetic DNA
agtctctagatccttcagaccaacgctgata

SEQ ID NO: 315 - DNA, 30 bp, Artificial Sequence
  Synthetic DNA
agtctctagatgacaggccgtttctatcaa

SEQ ID NO: 316 - DNA, 29 bp, Artificial Sequence
  Synthetic DNA
agtctctagagcctgcaatcaatccctgt

SEQ ID NO: 317 - DNA, 29 bp, Artificial Sequence
  Synthetic DNA
agtctctagattctgcggaaggaagctgt

SEQ ID NO: 318 - DNA, 30 bp, Artificial Sequence
  Synthetic DNA
agtctctagaagcggcagaaaatgagaaaa

SEQ ID NO: 319 - DNA, 33 bp, Artificial Sequence
  Synthetic DNA
agtctctagacaaagcaattagagtgaacgaca

SEQ ID NO: 320 - DNA, 30 bp, Artificial Sequence
  Synthetic DNA
agtctctagaagcacaggcacaacgtttac

SEQ ID NO: 321 - DNA, 30 bp, Artificial Sequence
  Synthetic DNA
agtctctagacggtaccaagggccattttg

SEQ ID NO: 322 - DNA, 30 bp, Artificial Sequence
  Synthetic DNA
agtctctagaaagatgtcgggaaagtgtcg

SEQ ID NO: 323 - DNA, 30 bp, Artificial Sequence
  Synthetic DNA
agtctctagattggcttgccttatcattcg

SEQ ID NO: 324 - DNA, 29 bp, Artificial Sequence
  Synthetic DNA
agtctctagagacgacttgcttgctcact

SEQ ID NO: 325 - DNA, 30 bp, Artificial Sequence
  Synthetic DNA
agtctctagatgctgcgacacgaatttcta

SEQ ID NO: 326 - DNA, 31 bp, Artificial Sequence
  Synthetic DNA
agtctctagagaggagggaaacaccgttaat

SEQ ID NO: 327 - DNA, 29 bp, Artificial Sequence
  Synthetic DNA
agtctctagacccttccaattgaggaaaa

SEQ ID NO: 328 - DNA, 30 bp, Artificial Sequence
  Synthetic DNA
agtctctagatgcaatgatatcgtcagcag

SEQ ID NO: 329 - DNA, 30 bp, Artificial Sequence
  Synthetic DNA
agtctctagacacctggattttgccttcag

SEQ ID NO: 330 - DNA, 29 bp, Artificial Sequence
  Synthetic DNA
agtctctagacttccatggtaaaggagca

SEQ ID NO: 331 - DNA, 30 bp, Artificial Sequence
  Synthetic DNA
agtctctagattctgcagcaacaacagctt

SEQ ID NO: 332 - DNA, 30 bp, Artificial Sequence
  Synthetic DNA
agtctctagaagcttcttgaggcgctacag

SEQ ID NO: 333 - DNA, 31 bp, Artificial Sequence
  Synthetic DNA
agtctctagatccagaggctgtggaaactgt

SEQ ID NO: 334 - DNA, 29 bp, Artificial Sequence
  Synthetic DNA
agtctctagaagcatgaggctgtatgagg

SEQ ID NO: 335 - DNA, 32 bp, Artificial Sequence
  Synthetic DNA
agtctctagatgcttgtagaagagcagaatcg

SEQ ID NO: 336 - DNA, 31 bp, Artificial Sequence
  Synthetic DNA
agtctctagacccagatgaagtcactggaac

SEQ ID NO: 337 - DNA, 31 bp, Artificial Sequence
  Synthetic DNA
agtctctagaacggatttcttcctggtgtct

SEQ ID NO: 338 - DNA, 31 bp, Artificial Sequence
  Synthetic DNA
agtctctagatgatatgctcccattctttcg

SEQ ID NO: 339 - DNA, 31 bp, Artificial Sequence
  Synthetic DNA
agtctctagatccttcgtatacgccagtacc

SEQ ID NO: 340 - DNA, 30 bp, Artificial Sequence
  Synthetic DNA
agtctctagagcatccacgtttgttccatt

SEQ ID NO: 341 - DNA, 30 bp, Artificial Sequence
  Synthetic DNA
agtctctagaagcattccctgtaccagacc

SEQ ID NO: 342 - DNA, 30 bp, Artificial Sequence
  Synthetic DNA
agtctctagaagccgaacgagctaagacat

SEQ ID NO: 343 - DNA, 30 bp, Artificial Sequence
  Synthetic DNA
agtctctagagaccaaagctccctcttgaa

SEQ ID NO: 344 - DNA, 30 bp, Artificial Sequence
  Synthetic DNA
agtctctagaggcatacgcctgtaattgct

SEQ ID NO: 345 - DNA, 27 bp, Artificial Sequence
  Synthetic DNA
agtctctagattcttcagggccagcaa

SEQ ID NO: 346 - DNA, 30 bp, Artificial Sequence
  Synthetic DNA
agtctctagaaggaaagcctatcgcacaca

SEQ ID NO: 347 - DNA, 31 bp, Artificial Sequence
  Synthetic DNA
agtctctagaagaccaactcgtcccattttc

SEQ ID NO: 348 - DNA, 30 bp, Artificial Sequence
  Synthetic DNA
agtctctagagtgggctttggttacatcgt

SEQ ID NO: 349 - DNA, 30 bp, Artificial Sequence
  Synthetic DNA
agtctctagaagttccttgcgtggatgttt

SEQ ID NO: 350 - DNA, 31 bp, Artificial Sequence
  Synthetic DNA
agtctctagacatccgatctgctttctcttg

SEQ ID NO: 351 - DNA, 30 bp, Artificial Sequence
  Synthetic DNA
agtctctagaatgggagagagcttcttgct

SEQ ID NO: 352 - DNA, 31 bp, Artificial Sequence
  Synthetic DNA
agtctctagagatctccagcttaagggttgc

SEQ ID NO: 353 - DNA, 31 bp, Artificial Sequence
  Synthetic DNA
agtctctagaagggttctcctcaagttcatc

SEQ ID NO: 354 - DNA, 30 bp, Artificial Sequence
  Synthetic DNA
agtctctagacgttgcagagttcgttgttc

SEQ ID NO: 355 - DNA, 30 bp, Artificial Sequence
  Synthetic DNA
agcttctagacagaacaacgtctcctacgc

SEQ ID NO: 356 - DNA, 30 bp, Artificial Sequence
  Synthetic DNA
agcttctagaagccaaacgcatagcttcat

SEQ ID NO: 357 - DNA, 30 bp, Artificial Sequence
  Synthetic DNA
agtctctagatagaagttgcaggcgttcct

SEQ ID NO: 358 - DNA, 32 bp, Artificial Sequence
  Synthetic DNA
agtctctagatccgtagttgttacggaaggtt

SEQ ID NO: 359 - DNA, 30 bp, Artificial Sequence
  Synthetic DNA
agtctctagatttcttggctccctgcatag

SEQ ID NO: 360 - DNA, 30 bp, Artificial Sequence
  Synthetic DNA
agtctctagacgcagaatgggataggacat

SEQ ID NO: 361 - DNA, 30 bp, Artificial Sequence
  Synthetic DNA
agtctctagattgcacaccttggacgtact

SEQ ID NO: 362 - DNA, 34 bp, Artificial Sequence
  Synthetic DNA
agtctctagatttcgttaattctcccttagacca

SEQ ID NO: 363 - DNA, 30 bp, Artificial Sequence
  Synthetic DNA
agtctctagaaagtttctgtgtcgctacgg

SEQ ID NO: 364 - DNA, 31 bp, Artificial Sequence
  Synthetic DNA
agtctctagaagaaaaatccatgggctgtaa

SEQ ID NO: 365 - DNA, 30 bp, Artificial Sequence
  Synthetic DNA
agtctctagattcatcggctgttgctgtat

SEQ ID NO: 366 - DNA, 30 bp, Artificial Sequence
  Synthetic DNA
agtctctagagaatacggaaagcgcaacat

SEQ ID NO: 367 - DNA, 27 bp, Artificial Sequence
  Synthetic DNA
atcgtaccatggagcaacccaattgtg

SEQ ID NO: 368 - DNA, 38 bp, Artificial Sequence
  Synthetic DNA
atcgtaccatggaattaaataaaacatccgagtctttg

SEQ ID NO: 369 - DNA, 33 bp, Artificial Sequence
  Synthetic DNA
atcgtaccatggttctcgcttcttgtcttcttg

SEQ ID NO: 370 - DNA, 35 bp, Artificial Sequence
  Synthetic DNA
atcgtaccatggatgaaggtattcaaaccgtttct

SEQ ID NO: 371 - DNA, 33 bp, Artificial Sequence
  Synthetic DNA
atcgtaccatggggttctcttctccagcacttc

SEQ ID NO: 372 - DNA, 36 bp, Artificial Sequence
  Synthetic DNA
atcgtaccatggagaacaaaacactaagcgttttct

SEQ ID NO: 373 - DNA, 31 bp, Artificial Sequence
  Synthetic DNA
agtcggtaccagcttccttaacttcgctgag

SEQ ID NO: 374 - DNA, 32 bp, Artificial Sequence
  Synthetic DNA
agtcggtaccttggttttgttcttgatcttgc

SEQ ID NO: 375 - DNA, 37 bp, Artificial Sequence
  Synthetic DNA
agtcggtaccatctaagaaatcatcttctgtaaggac

SEQ ID NO: 376 - DNA, 33 bp, Artificial Sequence
  Synthetic DNA
agtcggtaccgtctctaggaactagcgtttcca

SEQ ID NO: 377 - DNA, 35 bp, Artificial Sequence
  Synthetic DNA
agtcggtaccaatgtttgctatcaaacttacaatt

SEQ ID NO: 378 - DNA, 29 bp, Artificial Sequence
  Synthetic DNA
agtcggtacccaatgaaaatagaggcgga

(19) United States
(12) Patent Application Publication
(10) Pub. No.: US 2004/0208890 A1
(43) Pub. Date: Oct. 21, 2004

(54) Secreted chlamydia polypeptides, polynucleotides coding therefor, therapeutic and diagnostic uses thereof

(75) Inventors: Agathe Subtil-Sands, Paris (FR); Alice Dautry-Varsat, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT PASTEUR, Paris (FR)

(21) Appl. No.: 10/784,880

(22) Filed: Feb. 24, 2004

Related U.S. Application Data
(60) Provisional application No. 60/448,879, filed on Feb. 24, 2003.

(51) Int. Cl. C07K 14/295; C07H 21/04; C12P 21/04; A61K 39/02; C12N 1/21

(52) U.S. Cl. 424/190.1; 530/350; 435/69.3; 435/252.3; 435/320.1; 536/23.7

(57) ABSTRACT

The present invention relates to secreted Chlamydia polypeptides, which may be expressed by a Gram-negative bacterial strain and secreted by the type III secretion pathway of said bacterial strain. The present invention also relates to polynucleotides coding for these polypeptides, as well as to the therapeutic and vaccination uses of these secreted Chlamydia polypeptides.

### CROSS-REFERENCE TO RELATED APPLICATIONS

The present application claims priority to U.S. Provisional Application 60/448,879, filed on Feb. 24, 2003; the entire contents of which are incorporated herein by reference.

### FIELD OF THE INVENTION

The present invention relates to secreted Chlamydia polypeptides. More particularly, the present invention relates to secreted Chlamydia polypeptides expressed by a Gram-negative bacterial strain and secreted by the type III secretion pathway of said bacterial strain. The present invention also relates to the polynucleotides coding for these secreted Chlamydia polypeptides, as well as to the therapeutic, including vaccination, and diagnostic uses of these secreted Chlamydia polypeptides.

### BACKGROUND OF THE INVENTION

Chlamydiae are gram-negative bacteria that proliferate only within eukaryotic host-cells. The three species pathogenic to humans, Chlamydia trachomatis, Chlamydia psittaci and Chlamydia pneumoniae, cause a number of diseases, including trachoma, sexually transmitted disease, pelvic inflammatory disease, respiratory diseases, such as bronchitis, pneumonia and their sequelae (Gregory, D. W. and W. Schaffner. (1997). Psittacosis. Seminars in Respiratory Infections. 12: 7-11; Kuo, C.-C., L. Jackson, L. Campbell, and J. Grayston. (1995). Chlamydia pneumoniae (TWAR). Clin. Microbiol. Rev. 8: 451-61; Stamm, W. E. (1999). Chlamydia trachomatis infections: progress and problems. J. Infect. Dis. 179: S380-3). In addition, Chlamydia psittaci and Chlamydia pecorum have been demonstrated to be pathogenic to animals other than humans, and as such have a significant impact on agricultural economics. In particular, Chlamydia psittaci and Chlamydia pecorum have been causatively linked to abortion and pneumonitis in cattle, pig, and sheep (Fukushi et al. (1993). Microbiol Immunol. 37(7): 516-522; Tanner et al. (1999). Chlamydia: Intercellular Biology, Pathogenesis, and Immunity. Chapter 1).

During its cycle, Chlamydia adopts two distinct morphologies: a small infectious form, the elementary body (EB, 0.3 μm in diameter), and a larger replicative form, the reticulate body (RB, 1 μm in diameter) (Moulder, J. W. (1991). Interaction of chlamydiae and host cells in vitro. Microbiol. Rev. 55: 143-90.). EBs are adapted for survival in the extracellular space: their outer membrane is made rigid by a network of disulfide bonds and the bacteria are metabolically inactive. They initiate infection by attaching to a suitable host cell, principally epithelial cells of the mucosa, and they are internalized by a mechanism resembling phagocytosis (Boleti, H., A. Benmerah, D. Ojcius, N. Cerf-Bensussan, and A. Dautry-Varsat. (1999). Chlamydia infection of epithelial cells expressing dynamin and Eps 15 mutants: clathrin-independent entry into cells and dynamin-dependent productive growth. J. Cell. Sci. 112: 1487-1496). Within a few hours after internalization, EBs differentiate into replicative RBs, which proliferate in a growing vacuole and produce up to about a thousand progeny. After 2 to 3 days of infection RBs differentiate back into EBs, which are delivered to the extracellular space, and a new infectious cycle can begin.

Throughout their cycle in the host cell, Chlamydiae remain in a membrane-bound compartment called an inclusion. Bacteria escape from host defense mechanisms (i.e., cytosolic proteases or lysosomal enzymes) by preventing fusion of the inclusion with acidic degradative compartments of host cells. In addition, most bacterial antigens avoid detection by the host immune system, since these antigens do not access the infected cell plasma membrane. Inhibition of phagolysosome fusion is limited to Chlamydia psittaci-laden vacuoles. (Eissenberg, L. G., and P. B. Wyrick. (1981) Infect. Immun. 32: 889-896; Friis, R. R. (1972). Interaction of L cells and Chlamydia psittaci: entry of the parasite and host responses to its development. J. Bacteriol. 110: 706-721; Heinzen, R. A., M. A. Scidmore, D. D. Rockey, and T. Hackstadt. (1996). Differential interaction with endocytic and exocytic pathways distinguish parasitophorous vacuoles of Coxiella burnetii and Chlamydia trachomatis. Infect. Immun. 64: 769-809; Schramm, N., C. R. Bagnell, and P. B. Wyrick. (1996). Vesicles containing Chlamydia trachomatis serovar L2 remain above pH 6 within HEC-1B cells. Infect. Immun. 64:1208-1214). In fact, Chlamydiae seem to exchange very little with the endocytic compartments (Taraska, T., D. M. Ward, R. S. Ajioka, P. B. Wyrick, S. R. Davis-Kaplan, C. H. Davis, and J. Kaplan. (1996). The late chlamydial inclusion membrane is not derived from the endocytic pathway and is relatively deficient in host proteins. Infect. Immun. 64: 3713-372; Van Ooij, C., G. Apodaca, and J. Engel. (1997). Characterization of the Chlamydia trachomatis vacuole and its interaction with the host endocytic pathway in HeLa cells. Infect. Immun. 65: 758-766).

Yet, during their development cycle, the volume of the inclusions increases considerably, until they occupy a large portion of the cytosol. Part of the lipids necessary for this growth come from the host cell (Hatch, G. M., and G. McClarty. (1998). Phospholipid composition of purified Chlamydia trachomatis mimics that of the eucaryotic host cell. Infection & Immunity. 66: 3727-35). Sphingomyelin, synthesized in the Golgi apparatus, is transported to the inclusion membrane and incorporated into bacteria (Hackstadt, T., M. A. Scidmore, and D. D. Rockey. (1995). Lipid metabolism in Chlamydia trachomatis-infected cells: directed trafficking of Golgi-derived sphingolipids to the chlamydial inclusion. Proc. Natl. Acad. Sci, USA. 92: 4877-4881).

Surprisingly, no proteins of eukaryotic origin have been found associated with the inclusion. However, a number of prokaryotic proteins have been localized on the membrane of the inclusion, by fluorescence microscopy using antibodies generated against a variety of chlamydial proteins ( Bannantine, J. P., R. S. Griffiths, W. Viratyosin, W. J. Brown, and D. D. Rockey. (2000). A secondary structure motif predictive of protein localization to the chlamydial inclusion membrane. Cell. Microbiol. 2: 35-47; Bannantine, J. P., D. D. Rockey, and T. Hackstadt. (1998). Tandem genes of Chlamydia psittaci that encode proteins located to the inclusion membrane. Mol. Microbiol. 28: 1017-26; Rockey, D. D., R. A. Heinzen, and T. Hackstadt. (1995). Cloning and characterization of a Chlamydia psittaci gene coding for a protein localized in the inclusion membrane of infected cells. Mol. Microbiol. 15: 617-626; Scidmore-Carlson, M. A., E. I. Shaw, C. A. Dooley, E. R. Fischer, and T. Hackstadt. (1999). Identification and characterization of a Chlamydia trachomatis early operon encoding four novel inclusion membrane proteins. Mol. Microbiol. 33: 753-765). These proteins show no sequence similarities but have in common the presence of a large (40 to 70 amino acids) hydrophobic region and have therefore been grouped into a structural family called the Inc family.

The three first Inc proteins identified, IncA, IncB and IncC, are probably major components of the inclusion since they are recognized by antisera from convalescent guinea pigs infected with C. psittaci (Bannantine, J., W. Stamm, R. Suchland, and D. Rockey. (1998). Chlamydia trachomatis IncA is localized to the inclusion membrane and is recognized by antisera from infected humans and primates. Infect. Immun. 66: 6017-6021; Rockey, D. D., R. A. Heinzen, and T. Hackstadt. (1995). Cloning and characterization of a Chlamydia psittaci gene coding for a protein localized in the inclusion membrane of infected cells. Mol. Microbiol. 15: 617-626). They have homologs encoded by C. trachomatis and C. pneumoniae genomes, exhibiting about 20% global sequence identity and up to 50% sequence identity in the regions of highest similarity.

Inc proteins have been grouped into a family on two criteria: they have a large (larger than 40 residues) hydrophobic domain and they localize to the membrane of the inclusion in the host cell. The demonstration that several proteins encoded by the C. trachomatis genome and one protein from C. pneumoniae belong to this family was based on their localization to the membrane of the inclusion by immunofluorescence using specific antibodies (Bannantine, J., W. Stamm, R. Suchland, and D. Rockey. (1998). Chlamydia trachomatis IncA is localized to the inclusion membrane and is recognized by antisera from infected humans and primates. Infect. Immun. 66: 6017-6021;Bannantine, J. P., R. S. Griffiths, W. Viratyosin, W. J. Brown, and D. D. Rockey. (2000). A secondary structure motif predictive of protein localization to the chlamydial inclusion membrane. Cell. Microbiol. 2: 35-47; Bannantine, J. P., D. D. Rockey, and T. Hackstadt. (1998). Tandem genes of Chlamydia psittaci that encode proteins located to the inclusion membrane. Mol. Microbiol. 28: 1017-26; Rockey, D. D., R. A. Heinzen, and T. Hackstadt. (1995). Cloning and characterization of a Chlamydia psittaci gene coding for a protein localized in the inclusion membrane of infected cells. Mol. Microbiol. 15: 617-626.; Scidmore-Carlson, M. A., E. I. Shaw, C. A. Dooley, E. R. Fischer, and T. Hackstadt. (1999). Identification and characterization of a Chlamydia trachomatis early operon encoding four novel inclusion membrane proteins. Mol. Microbiol. 33: 753-765). However, the mechanism of their secretion and insertion into the membrane of the inclusion had not been investigated yet, due to the lack of genetic tools for members of Chlamydia spp.

How do chlamydial proteins reach the membrane of the inclusion? Four pathways of protein secretion (types I to IV) have been described in Gram-negative bacteria. Type III secretion machines allow for the translocation of bacterial effectors into the eukaryotic cell cytosol during bacterial contact with the cell surface (Hueck, C. (1998). Type III protein secretion systems in bacterial pathogens of animals and plants. Microbiol. Mol. Biol. Rev. 62: 379-433). The inventors hypothesized that Chlamydiae use type III secretion to insert proteins into the membrane of the inclusion (Subtil A., A. Blocker, and A. Dautry-Varsat. (2000). Type III secretion system in Chlamydia: identified members and candidates. Microbes Infect. 2: 367-369). Presence of genes encoding putative components of a type III secretion machinery in the Chlamydia genome suggested that Inc proteins might be secreted by a type III secretion pathway since type III secretion is used by a large number of Gram-negative pathogens for the insertion or the translocation of bacterial proteins into or through eukaryotic cell membranes. In the absence of genetic tools to manipulate the Chlamydia genome, it was difficult to test this hypothesis directly. Shigella uses a type III secretion system for entry into epithelial cells and for dissemination (Nhieu, G. T., and P. J. Sansonetti. (1999). Mechanism of Shigella entry into epithelial cells. Curr. Opin. Microbiol. 2: 51-5; Page, A.-L., H. Ohayon, P. J. Sansonetti, and C. Parsot. (1999). The secreted IpaB and IpaC invasins and their cytoplasmic chaperone IpgC are required for intercellular dissemination of Shigella flexneri. Cell. Microbiol. 1: 183-193; Schuch, R., R. C. Sandlin, and A. T. Maurelli. (1999). A system for identifying post-invasion functions of invasion genes: requirements for the Mxi-Spa type III secretion pathway of Shigella flexneri in intercellular dissemination, Mol. Microbiol. 34:675-689). The secretion signal involved in secretion of proteins by the type III secretion pathway is not known. However, it is often located either within the first fifteen codons of the mRNA coding for the secreted proteins, or within the N-terminal part of the secreted protein (Anderson, D. M., and O. Schneewind. (1999). Type III machines of Gram-negative pathogens: injecting virulence factors into host cells and more. Curr. Opin. Microbiol. 2: 18-24). Although the genomes of several Chlamydia species are now available, it is not possible to identify secreted proteins on the basis of their amino acid sequence.

Genes coding for the type III secretion system in Shigella are known (Hueck, C. (1998). Type III protein secretion systems in bacterial pathogens of animals and plants. Microbiol. Mol. Biol. Rev. 62: 379-433) and it has been hypothesized that Inc proteins may be secreted by a similar machinery.

Expression of heterologous proteins by use of type III machinery of Shigella has already been obtained (see “Functional conservation of the Salmonella and Shigella effectors of entry into epithelial cells” published in Molecular Microbiology (1995) 17 (4), 781-789, Hermant, Ménard, Arricau, Parsot and Popoff). However, as indicated in the title, this document relates to Salmonella proteins, which is different from the Chlamydia proteins due to the phylogenic distance of Chlamydia from other organisms (see page 369, first column of Subtil, et al.; Microbes and Infection 2, 2000; Subtil A., A. Blocker, and A. Dautry-Varsat. (2000). Type III secretion system in Chlamydia: identified members and candidates. Microbes Infect. 2: 367-369).

Furthermore, methods for screening inhibitors and activators of type III secretion machinery in gram-negative bacteria, in Shigella, have already been disclosed in WO 99/58714.

Several gram-negative pathogens, including chlamydiae, possess a type III secretion machinery for the insertion or the translocation of bacterial proteins into or through eukaryotic membranes. In U.S. patent application Ser. No. 10/014,670 (the entire disclosure of which is incorporated herein by reference), the present inventors constructed chimeras by fusing the N-terminal part of these proteins with a reporter gene, the Cya protein of Bordetella pertussis, and expressed these chimeras in various strains of Shigella flexneri. The use of a reporter gene is well known by those of ordinary skill in the art. The reporter sequence, which codes for an easily detectable protein, is linked to a fragment of the DNA of interest. The use of the B. pertussis cya gene as a reporter gene, chosen in the present invention, is reported by: Jones et al., 1998 and Sory and Cornelis, 1994 (Jones, M. A., M. W. Wood, P. B. Mullan, P. R. Watson, T. S. Wallis, and E. E. Galyov. (1998). Secreted effector proteins of Salmonella dublin act in concert to induce enteritis. Infection & Immunity. 66: 5799-804; Sory, M. P., and G. R. Cornelis. (1994). Translocation of a hybrid YopE-adenylate cyclase from Yersinia enterocolitica into HeLa cells. Mol. Microbiol. 14: 583-94). Moreover, the inventors have shown that several chlamydial proteins, including members of the Inc family and proteins selected for a hydropathic profile similar to that of Inc proteins, are secreted by the type III secretion machinery of S. flexneri. Identification of such proteins is very important since these proteins may be involved in functions essential for the intracellular survival of Chlamydia. They may serve as a basis for the development of a chlamydiae vaccine or a treatment against Chlamydia infection, by suggesting non-immune therapeutic interventions capable of inhibiting bacterial development, as well as for the development of Chlamydia infection diagnosis.

A need continues to exist, however, for further elucidation of chlamydial polypeptides that are secreted, as well as the diagnostic and therapeutic uses of the same.

In the present invention, the inventors describe the identification of several new proteins secreted by Chlamydia spp. during infection. Most of these proteins are from the human pathogens C. trachomatis and C. pneumoniae, and a few proteins are from one strain of the human and animal pathogen C. psittaci. In several cases the inventors have shown that if a protein is secreted in one species, homologous proteins from other species are also secreted (see below, category 1). Therefore knowledge that a protein is secreted in one species gives a good indication that it is also true in another species and this can be checked with the method described herein. Thus, the results presented here can also be extended to different Chlamydia species, to be used as a basis for the development of a chlamydiae vaccine or a treatment against Chlamydia infection, as well as for the development of Chlamydia infection diagnosis, in humans or in animals.

### SUMMARY OF THE INVENTION

It is an object of the present invention to provide a purified secreted polypeptide from Chlamydia spp., e.g., Chlamydia pneumoniae, Chlamydia trachomatis, Chlamydia pecorum, or Chlamydia psittaci. The purified polypeptide may be selected by a method comprising (a) providing a recombinant expression vector containing at least a polynucleotide coding for the polypeptide of interest; (b) transforming a Gram-negative strain containing a type III secretion pathway with said recombinant vector; (c) expressing this vector in the Gram-negative strain transformed in (b); and (d) detecting the secretion of said polynucleotide expression product; wherein the secretion of said expression product indicates that it corresponds to a secreted Chlamydia polypeptide. The detection of the secretion of expression product is made by detecting the presence of said product outside the bacteria.

Alternatively, the purified polypeptide may be selected by a method comprising (a) providing a recombinant expression vector comprising at least a DNA coding for the polypeptide of interest fused to a reporter gene (i.e., the recombinant expression vector comprises a fusion construct in which one component of the construct is the polynucleotide of interest and a second component of the construct is the polynucleotide sequence of a reporter gene); (b) transforming a Gram-negative strain containing a type III secretion pathway with said recombinant vector; (c) expressing this vector in the Gram-negative strain transformed in (b); and (d) detecting the secretion of said reporter gene expression product; wherein the secretion of said expression product indicates that the fused DNA (i.e., the fusion construct of (a), which contains the polynucleotide of interest and a polynucleotide sequence of a reporter gene) contains at least a polynucleotide corresponding to a secreted Chlamydia polypeptide. The detection of the secretion of expression product is performed by detecting the presence of said product outside the bacteria.

Another object of the present invention is to provide a purified polynucleotide coding for at least one polypeptide that is secreted from Chlamydia.

Another object of the present invention is to provide an immunogenic composition comprising a secreted Chlamydia polypeptide, or an immunogenic fragment thereof.

Another object of the present invention is to provide a vaccinating composition against Chlamydia infection comprising a secreted Chlamydia polypeptide, or an immunogenic fragment thereof. Said composition is a human and/or a veterinary vaccinating composition. For example, said infection is a sexually transmitted disease , respiratory disease, such as pneumonia or bronchitis, or contributes to atherosclerosis.

Another object of the present invention is to provide a therapeutic composition active against Chlamydia infection comprising a molecule, which inhibits the secretion of a secreted Chlamydia polypeptide.

Another object of the present invention is to provide a complex comprising a secreted Chlamydia polypeptide and an antibody directed against said polypeptide.

Another object of the present invention is to provide an antibody against Chlamydia wherein the antibody is directed against a secreted Chlamydia polypeptide, or an immunogenic fragment thereof, wherein said antibody is a monoclonal antibody or a polyclonal antibody.

Another object of the present invention is to provide methods for diagnosing a Chlamydia infection in an animal, including a human. A method for diagnosing a Chlamydia infection in an animal, comprises (a) providing a secreted polypeptide of Chlamydia, or an immunogenic fragment thereof, optionally labeled; (b) bringing said polypeptide or immunogenic fragment thereof into contact with a serum sample of said animal; and (c) detecting complexes formed between said polypeptide or immunogenic fragment thereof and antibodies contained in the serum sample; wherein said complexes are indicative of a Chlamydia infection in said animal.

Alternatively, a method for diagnosing a Chlamydia infection in an animal, including a human, comprises (a) providing an animal sample of a tissue suspected to be infected by Chlamydia; (b) bringing said sample into contact with an antibody against Chlamydia wherein said antibody is directed against a purified secreted polypeptide of Chlamydia; and (c) detecting antigen-antibody complexion; wherein said complexion is indicative of a Chlamydia infection in said animal.

Within the objects of the present invention, said purified polypeptides of Chlamydia may be identified by their expression and secretion in a Gram negative strain containing a type III secretion pathway.

In yet another object, the present invention provides a method of detecting Chlamydia in an animal, including a human, using a probe or primer pair selected from Table I or II. The method for detecting Chlamydia in an animal comprises (a) providing an animal sample of a tissue suspected to be infected by Chlamydia; (b) adding a primer pair selected from the list presented in Tables I and II to the tissue sample; (c) amplifying a polynucleotide that encodes for the protein which corresponds to the primer pair selected; and (d) detecting the presence of Chlamydia by the presence or absence of said polynucleotide.

In still another object of the present invention is a method of detecting Chlamydia in an animal, including a human, using a probe that hybridizes under stringent conditions with a polynucleotide that encodes a polypeptide of the present invention. The method for detecting Chlamydia in an animal comprises (a) providing an animal sample of a tissue suspected to be infected by Chlamydia; (b) adding a hybridization probe that hybridizes with a polynucleotide that encodes a polypeptide of the present invention under stringent conditions; and (c) detecting the presence of Chlamydia by the presence or absence of said polynucleotide.

In still another object of the present invention is a method of detecting Chlamydia in an animal, including a human, using an antibody, optionally labeled, that forms a complex with a polypeptide of the present invention. The method for detecting Chlamydia in an animal comprises (a) providing an animal sample of a tissue suspected to be infected by Chlamydia; (b) adding an antibody, optionally labeled, that forms a complex with a polypeptide of the present invention under conditions suitable for complex formation; and (c) detecting the presence of Chlamydia by the presence or absence of said polypeptide.

Another object of the present invention is to provide a recombinant plasmid for the expression of a secreted Chlamydia polypeptide.

Another object of the present invention is to provide a recombinant prokaryotic cell, for example a recombinant Gram-negative bacterial strain, transformed by a vector comprising at least a polynucleotide encoding a secreted Chlamydia polypeptide.

Another object of the present invention is to provide a method of preventing or treating a Chlamydia infection in an animal, including a human, which comprises administering an effective amount of a purified secreted polypeptide, or immunogenic fragment thereof, of Chlamydia of the present invention.

In still another object, the present invention provides a method of preventing or treating a Chlamydia infection in an animal, which comprises administering an effective amount of an antibody directed against a secreted Chlamydia polypeptide according to the present invention, or an immunogenic fragment thereof, to an animal in need thereof.

Still another object of the present invention is to provide a method of screening for an active molecule inhibiting the secretion of a secreted Chlamydia polypeptide, comprising (a) supplying an active molecule to a culture of Chlamydia; (b) growing or incubating said culture for a time and under conditions suitable for said active molecule to exert an activity upon said culture; (c) adding a primer pair for a Chlamydia polypeptide to the culture; (d) amplifying a polynucleotide that encodes for the polypeptide which corresponds to the primer pair selected; and (e) detecting the presence of the secreted Chlamydia polypeptide by the presence or absence of said polynucleotide.

A further object of the present invention is to provide a method of detecting the presence of a Chlamydia polypeptide by using a probe that hybridizes under stringent conditions with a polynucleotide that encodes the polypeptide or by using antibodies, optionally labeled, that form a complex with the polypeptide.

In yet another object of the present invention is to provide a method of screening for an active molecule inhibiting the secretion of a secreted Chlamydia polypeptide, comprising (a) supplying an active molecule to a culture of Chlamydia; (b) growing or incubating said culture for a time and under conditions suitable for said active molecule to exert an activity upon said culture; and (c) evaluating the presence of a polypeptide according to the present invention.

The above objects highlight certain aspects of the invention. Additional objects, aspects and embodiments of the invention are found in the following detailed description of the invention.

### BRIEF DESCRIPTION OF THE DRAWINGS

A more complete appreciation of the invention and many of the attendant advantages thereof will be readily obtained as the same becomes better understood by reference to the following Figures in conjunction with the detailed description below.

FIG. 1: Illustration of the secretion assay in Shigella. The ipaB mutant strain of Shigella constitutively secretes high level of effectors by the type III pathway. This strain was transformed with different chimeric constructs and secretion of the chimeras was probed on colonies. Colonies expressing the different constructs were picked on a fresh LB plate in the morning and incubated for 8 hours at 37° C. The colonies were then covered with a PVDF membrane and grown overnight at 37° C. Secretion of the chimera was detected on this membrane by Western blot using anti-cyclase antibody. Secreted proteins formed a halo around the colonies (2 lower rows) while non-secreted proteins were detected only on the spot where the colonies had grown (2 upper rows).

FIG. 2: Full length chlamydial proteins are secreted by Shigella. The ipaB mutant strain of Shigella constitutively secretes a high level of effectors by the type III pathway, while the mxiD mutant strain is totally deficient for type III secretion. In this experiment, the strains were transformed with Psi1058 and the presence of the protein in the culture supernatant was probed by Western blot. Psi1058 was found in the supernatant (S) fraction of the ipaB stain culture. In the mxiD strain it was found only associated with the pellet (P) fraction. This result shows that Psi1058 is secreted by a type III mechanism in S. flexneri. The same result was found with S. flexneri transformed with CPn0175, Psi0705, Psi725, Psi774, and Psi1005.

FIG. 3: HeLa cells were infected or not for 24 hours with C.psittaci GPIC strain, scrapped and broken by 7 passages through a 25G^{1/2} syringe needle in homogenization buffer (0.25 M sucrose, 0.1% gelatine, 0.5 mM EGTA, 3 mM imidazole pH 7.4). The cells were centrifuged for 15 minutes at 800 g, the pellet was resuspended in gel sample buffer (pellet 1 contains unbroken cells, bacteria and cell nuclei) and the supernatant was centrifuged for 45 minutes at 541 000 g (pellet 2 contains cellular membranes and bacteria, the supernatant contains proteins present in the cytosol). Equal fractions of pellets and supernatant were analyzed by electrophoresis in 8% polyacrylamide gels in the presence of sodium dodecyl sulfate (SDS-PAGE). After electrophoresis, proteins were transferred on a PVDF membrane (Millipore Corporation, Bedford, Mass.), and the membrane was used for blotting with anti-Psi0705 antibody. Western blotting and revelation were performed by enhanced chemiluminescence (Amersham Pharmacia Biotech) according to the manufacturer's instructions. Psi0705 was present in the pellet fractions as well as in the supernatant fraction (cytosolic material). As a control MOMP (major outer membrane protein) was present only in the pellet fractions.

FIG. 4: HeLa cells were infected for 24 hours with C.psittaci GPIC strain, fixed and permeablized with 0.05% saponin. Psi0710 was labeled with specific rabbit antibodies raised against the purified protein, followed by anti-rabbit Alexa488-coupled secondary antibodies (Molecular Probes). Cells were examined under an epifluorescence microscope attached to a cooled CCD-camera. Arrowheads point to fibers extending from the membrane of inclusion, to which Psi0710 is associated.

### DETAILED DESCRIPTION OF THE INVENTION

Unless specifically defined, all technical and scientific terms used herein have the same meaning as commonly understood by a skilled artisan in biochemistry, molecular biology, enzymology, genetics, immunology, general medicine and veterinary medicine.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

A “polypeptide” as used herein is understood to mean a sequence of several amino acid residues linked by peptide bonds. Such amino acids are known in the art and encompass the unmodified and modified amino acids. In addition, one or more modifications known in the art such as glycosylation, phosphorylation, etc may modify the polypeptide.

The term “secreted Chlamydia polypeptide” as used herein is understood to mean a Chlamydia protein, or fragment of the protein, comprising at least 20 amino acids, detectable outside the bacteria.

The term “homologous” as used herein is understood to mean two or more proteins of Chlamydia from the same species or from a different species. Within the meaning of this term, said two or more proteins share at least 40% identity to a fragment having at least 50 amino acids. Preferably, homologous Chlamydia proteins share at least 50% identity to a fragment having at least 50 amino acids. More preferably, homologous Chlamydia proteins share at least 60% identity, at least 70% identity, at least 80% identity, at least 90% identity, at least 95% identity, or 100% identity to a fragment having at least 50 amino acids. Accordingly, homologous Chlamydia proteins are included within the scope of the present invention.

The term “outside the bacteria” as used herein is understood to mean that a certain portion of the polypeptide of the invention produced by the bacteria has crossed the inner membrane, the periplasm and the outer membrane of the bacteria and is either still bound to the outer membrane, found in the extracellular medium or found inside the host cell.

As used herein, the term “active molecule inhibiting the secretion of a secreted Chlamydia polypeptide” is understood to mean a molecule able to reduce, including to totally remove, said secretion by any means. For example, said reduction, including total removal, of secretion may result from an action of said molecule on the type III secretion system or on the expression of said polypeptide by the bacteria.

The term “type III secretion pathway” as used herein is understood to mean a complex association of various molecules, which are necessary for secretion of a polypeptide in a host in which it is normally expressed, as described in Hueck C J. (1998). Type III protein secretion systems in bacterial pathogens of animals and plants. Microbiol Mol Biol Rev. 62: 379-433.

The term “immunogenic fragment” as it relates to polypeptides is understood to mean a polypeptide fragment of at least 6 amino acids and preferably at least 10 amino acids sufficient to induce an immune response when it is administered to a host eukaryotic organism.

The term “heterologous” as it relates to protein or polypeptide secretion systems is understood to mean that the protein or polypeptide is not normally expressed in a host.

Nucleic acids can be detected utilizing a nucleic acid amplification technique, such as polymerase chain reaction (PCR). Alternatively, the nucleic acid can be detected utilizing direct hybridization or by utilizing a restriction fragment length polymorphism.

Hybridization protocols are known in the art and are disclosed, for example, in Sambrook et al., Molecular Cloning: A Laboratory Manual, Cold Spring Harbor Laboratory, New York (1989).

The term “Chlamydia infection” as used herein means an infection in an animal, especially a human, that is caused by a species of Chlamydia; i.e., Chlamydia trachomatis, Chlamydia psittaci, Chlamydia pneumoniae and Chlamydia pecorum. Within the context of the present invention, a Chlamydia infection causes a number of diseases, including trachoma, sexually transmitted disease, pelvic inflammatory disease, respiratory diseases, such as bronchitis, pneumonia and their sequelae, such as atherosclerosis.

As used herein, stringent hybridization conditions are those conditions which allow hybridization between polynucleotides that are 75%, 80%, 85%, 90%, 95%, or 98% homologous as determined using conventional homology programs, an example of which is UWGCG sequence analysis program available from the University of Wisconsin. (Devereaux et al., Nucl. Acids Res. 12: 387-397 (1984)). Such stringent hybridization conditions typically include washing the hybridization reaction mixture in 2×SSC and 0.5% SDS at 65° C. (Sambrook et al., Molecular Cloning: A Laboratory Manual, Cold Spring Harbor Laboratory, New York (1989)). Of course, one of skill in the art will recognize that conditions can be varied depending on the length of the polynucleotides to be hybridized and the GC content of the polynucleotides see, for example, Sambrook et al., Molecular Cloning: A Laboratory Manual, Cold Spring Harbor Laboratory, New York (1989).

In this invention “primer” or “probe” means a polynucleotide, especially an oligonuclotide, which is produced synthetically or biologically and includes a specific nucleotide sequence, which permits hybridization to a section containing the target nucleotide sequence. Also in the present invention, the phrase “primer pair selected from the list presented in Tables I and II” means a single forward primer and the corresponding reverse primer for the protein of interest.

Defined primers or probes, as well as all other oligonucleotides and polynuclotides of the present invention, may be produced by any of several well-known methods, including automated solid-phase chemical synthesis using cyanoethyl-phosphoramidite precursors. Other well-known methods for construction of synthetic primers/oligonucleotides may, of course, be employed. J. Sambrook, E. F. Fritsch and T. Maniatis, Molecular Cloning 11 (2d ed. 1989).

The primers used to amplify the sample nucleic acids may be coupled to a detectable moiety. A preferred example of such a detectable moiety is fluorescein, which is a standard label used in nucleic acid sequencing systems using laser light as a detection system. Other detectable labels can also be employed, however, including other fluorophores, radio labels, chemical couplers such as biotin which can be detected with streptavidin-linked enzymes, and epitope tags such as digoxigenin detected using antibodies. The primers may be modified whereby another nucleotide is added to, removed from, or substituted for at least one nucleotide in the oligonucleotide. Introduction of known labels such as radioactive substances, enzymes, fluorescence substances, etc. after synthesis of oligonucleotide is also included therein.

Similarly, the probes/oligonucleotides used to hybridize with the polynucleotides coding for the polypeptides of the invention, for example for the purpose of detection of such a polynucleotide, may be coupled to a detectable moiety.

Examples of suitable primers for use in the present invention are shown in Table I.

The inventors have constructed chimeras between the N-terminal domain of several chlamydial proteins and a reporter protein, the calmodulin-dependent adenylate cyclase (Cya) from B. pertussis, and have tested whether these chimeras were secreted by S. flexneri. Proteins analyzed in this study can be classified into five categories.

(i) Corresponding chimeras from C. pneumoniae, C. trachomatis, and C. psittaci that are homologous and are secreted. Sequence analysis has shown that these three species are rather distant, and, as a consequence, the percentage of amino acid identity of the N-terminal domain between homologous proteins is generally low. Consequently, the chimeras constructed from homologous proteins share little identity in their amino terminal region, which is the region containing the putative secretion signal. Therefore, the identification that the three homologous chimeras (one from each of the Chlamydia species above) are secreted strongly supports that the corresponding proteins are secreted during Chlamydia infection. This category comprises the following polypeptides: CPn0330; CT083;

Psi0330; CPn0379; CT053; Psi0379; CPn0595; CT476; Psi0595; CPn0648; CT529; Psi0648; CPn0671; CT550; Psi0671; CPn0710; CT666; Psi0710; CPn761; CT610; Psi0761; CPn0774; CT606.1; Psi0774; CPn1002; CT845; Psi1002; CPn1005; CT848; Psi1005; CPn1022; CT863; Psi1022.

(ii) Proteins of C. pneumoniae and C. trachomatis which are homologous and for which the 2 corresponding chimeras are secreted. For the same reasons as explained above in the case of category 1, these results support that these proteins are also secreted during Chlamydia infection. This category comprises the following polypeptides: CPn0490; CT387; CPn0705; CT671; CPn0711; CT665; CPn0712; CT664; CPn0725; CT652.1; CPn0770; CT642; CPn0859; CT718; CPn0906; CT763.

(iii) C. pneumoniae proteins that are secreted, which have no homolog in C. trachomatis or C. psittaci. They may be important proteins, being specific of C. pneumoniae. This category comprises the following polypeptides: CPn0009; CPn0012; CPn0028; CPn0049; CPn0063; CPn0066; CPn0067; CPn0130; CPn0132; CPn0146; CPn0167; CPn0174; CPn0175; CPn0181; CPn0210; CPn0211; CPn0220; CPn0223; CPn0226; CPn0243; CPn0267; CPn0277; CPn0284; CPn0357; CPn0365; CPn0585; CPn0829; CPn1027.

(iv) C. pneumoniae proteins that have a homolog in at least one of the other 2 sequenced Chlamydia species and for which secretion of the corresponding homologous chimera is undetermined. This category comprises the following polypeptides: CPn0026; CPn0104; CPn0186; CPn0206; CPn0291; CPn0292; CPn0405; CPn0443; CPn0480; CPn0489; CPn0556; CPn0673; CPn0681; CPn0720; CPn0746; CPn0853; CPn0879; CPn0939; CPn1019; CPn1020; CPn1032.

(v) C. pneumoniae proteins that have homologs in the other 2 sequenced Chlamydia species and for which secretion of the corresponding homologous chimeras are negative or undetermined. Although these results support that most proteins in this category are secreted by Chlamydia during infection, the Inventors regard them as somewhat less strong candidates than the proteins classified in the other 4 categories, until more data are obtained (see for example the result with Psi1058 below). This category comprises the following polypeptides: CPn0105; CPn0287; CPn0334; CPn0374; CPn0399; CPn0497; CPn0522; CPn0582; CPn0588; CPn0729; CPn0755; CPn0764; CPn0792; CPn0820; CPn0821; CPn1007; CPn1016; CPn1058.

The polypeptides of the invention are administered in a dose, which is effective to vaccinate an animal, preferably a human, against Chlamydial infection or to treat an animal, preferably a human, having a Chlamydial infection. As used herein, an effective amount of the polypeptides to achieve this goal is generally from about 2 ng to 2 mg/kg of body weight per week, preferably about 2 μg/kg per week. This range includes all specific values and subranges therebetween.

In a preferred embodiment, the Chlamydia polypeptide is homologous to one or more Chlamydia pneumoniae proteins selected from the group consisting of CPn0104 (SEQ ID NO: 2), CPn206 (SEQ ID NO: 4), CPn0210 (SEQ ID NO: 6), CPn0399 (SEQ ID NO: 8), CPn0405 (SEQ ID NO: 10), CPn0443 (SEQ ID NO: 12), CPn0480 (SEQ ID NO: 14), CPn0489 (SEQ ID NO: 16), CPn0490 (SEQ ID NO: 18), CPn0497 (SEQ ID NO: 20), CPn0522 (SEQ ID NO: 22), CPn0556 (SEQ ID NO: 24), CPn0582 (SEQ ID NO: 26), CPn0588 (SEQ ID NO: 28), CPn0595 (SEQ ID NO: 30), CPn0671 (SEQ ID NO: 32), CPn0673 (SEQ ID NO: 34), CPn0681 (SEQ ID NO: 36), CPn0712 (SEQ ID NO: 38), CPn0720 (SEQ ID NO: 40), CPn0725 (SEQ ID NO: 42), CPn0729 (SEQ ID NO: 44), CPn0746 (SEQ ID NO: 46), CPn0755 (SEQ ID NO: 48), CPn0761 (SEQ ID NO: 50), CPn0764 (SEQ ID NO: 52), CPn0770 (SEQ ID NO: 54), CPn0774 (SEQ ID NO: 56), CPn0792 (SEQ ID NO: 58), CPn0853 (SEQ ID NO: 60), CPn0859 (SEQ ID NO: 62), CPn0879 (SEQ ID NO: 64), CPn0906 (SEQ ID NO: 66), CPn0939 (SEQ ID NO: 68), CPn1002 (SEQ ID NO: 70), CPn1005 (SEQ ID NO: 72), CPn1007 (SEQ ID NO: 74), CPn1019 (SEQ ID NO: 76), CPn1020 (SEQ ID NO: 78), CPn1032 (SEQ ID NO: 80), and CPn1058 (SEQ ID NO: 82); or a fragment thereof.

In another embodiment, the homologous Chlamydia polypeptide is a Chlamydia trachomatis protein selected from the group consisting of CT387 (SEQ ID NO: 84), CT476 (SEQ ID NO: 86), CT550 (SEQ ID NO: 88), CT606.1 (SEQ ID NO: 90), CT610 (SEQ ID NO: 92), CT642 (SEQ ID NO: 94), CT652.1 (SEQ ID NO: 96), CT664 (SEQ ID NO: 98), CT718 (SEQ ID NO: 100), CT763 (SEQ ID NO: 102), CT845 (SEQ ID NO: 104), and CT848 (SEQ ID NO: 106); or a fragment thereof.

In still another embodiment, the homologous Chlamydia polypeptide is a Chlamydia psittaci protein selected from the group consisting of Psi0330 (SEQ ID NO: 108), Psi0379 (SEQ ID NO: 110), Psi0595 (SEQ ID NO: 112), Psi0648 (SEQ ID NO: 114), Psi0671 (SEQ ID NO: 116), Psi0705 (SEQ ID NO: 118), Psi0710 (SEQ ID NO: 120), Psi0761 (SEQ ID NO: 122), Psi0774 (SEQ ID NO: 124), Psi1002 (SEQ ID NO: 126), Psi1005 (SEQ ID NO: 128), Psi1022 (SEQ ID NO: 130), and Psi1058 (SEQ ID NO: 132); or a fragment thereof.

A Chlamydia infection in an animal, preferably a human, as used herein includes a number of diseases, including trachoma, sexually transmitted disease, pelvic inflammatory disease, respiratory diseases, such as bronchitis, pneumonia and their sequelae, such as atherosclerosis.

Examples of other animals envisioned within the purview of the present invention include: a horse (equine animal), a cow (bovine animal), a pig (porcine animal), a sheep (ovine animal), a goat (caprine animal), a bird, a dog, and a cat.

The polypeptides of the present invention may be administered as a pharmaceutical composition containing the polypeptide compound and a pharmaceutically acceptable carrier or diluent. The active materials can also be mixed with other active materials, which do not impair the desired action and/or supplement the desired action. Any route can administer the active materials according to the present invention, for example, orally, parenterally, intravenously, intradermally, subcutaneously, or topically, in liquid or solid form.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

Another mode of administration of the polypeptides of this invention is oral. Oral compositions will generally include an inert diluent or an edible carrier. For the purpose of oral therapeutic administration, the aforesaid polypeptides may be incorporated with excipients and used in the form of tablets, gelatine capsules, troches, capsules, elixirs, suspensions, syrups, wafers, chewing gums and the like. Compositions may be prepared according to any method known to the art for the manufacture of pharmaceutical compositions and such compositions may contain one or more agents selected from the group consisting of sweetening agents, flavoring agents, coloring agents and preserving agents. Tablets containing the active ingredient in admixture with nontoxic pharmaceutically acceptable excipients, which are suitable for manufacture of tablets, are acceptable. These excipients may be, for example, inert diluents, such as calcium carbonate, sodium carbonate, lactose, calcium phosphate or sodium phosphate granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques to delay disintegration and adsorption in the gastrointestinal tract and thereby provide a sustained action over a longer period. For example, a time delay material such as glyceryl monostearate or glyceryl distearate alone or with a wax may be employed. Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

The tablets, pills, capsules, troches and the like may contain the following ingredients: a binder such as microcrystalline cellulose, gum tragacanth or gelatin; an excipient such as starch or lactose, a disintegrating agent such as alginic acid, Primogel, corn starch and the like; a lubricant such as magnesium stearate or Sterotes; a glidant such as colloidal silicon dioxide; and a sweetening agent such as sucrose or saccharin or flavoring agent such as peppermint, methyl salicylate, or orange flavoring may be added. When the dosage unit form is a capsule, it may contain, in addition to material of the above type, a liquid carrier such as a fatty oil. Other dosage unit forms may contain other various materials, which modify the physical form of the dosage unit, for example, as coatings. Thus tablets or pills may be coated with sugar, shellac, or other enteric coating agents. A syrup may contain, in addition to the active polypeptides, sucrose as a sweetening agent and certain preservatives, dyes and colorings and flavors. Materials used in preparing these various compositions should be pharmaceutically or veterinarially pure and non-toxic in the amounts used.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropylethyl cellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension may also contain one or more preservatives such as ethyl or n-propyl p-hydroxybenzoate, one or more coloring agents, one or more flavoring agents and one or more sweetening agents, such as sucrose, aspartame, saccharin, or sucralose.

Oil suspensions may be formulated by suspending the active ingredient in a vegetable oil, such as arachis oil, olive oil, sesame oil or coconut oil, or in a mineral oil such as liquid paraffin. The oil suspensions may contain a thickening agent, such as beeswax, hard paraffin or cetyl alcohol. Sweetening agents may be added to provide a palatable oral preparation. These compositions may be preserved by the addition of an antioxidant such as ascorbic acid.

Dispersible powders and granules of the invention suitable for preparation of an aqueous suspension by the addition of water may be formulated from the active ingredients in admixture with a dispersing, suspending and/or wetting agent, and one or more preservatives. Suitable dispersing or wetting agents and suspending agents are exemplified by those disclosed above. Additional excipients, for example sweetening, flavoring and coloring agents, may also be present.

The compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The compositions of the invention may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a nontoxic parenterally acceptable diluent or solvent, such as a solution of 1,3-butanediol. Among the acceptable vehicles and solvents that may be employed are water and Ringer's solution, an isotonic sodium chloride. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Sterilization may be performed by conventional methods known to those of ordinary skill in the art such as by aseptic filtration, irradiation or terminal sterilization (e.g. autoclaving).

Aqueous formulations (i.e oil-in-water emulsions, syrups, elixers and injectable preparations) may be formulated to achieve the pH of optimum stability. The determination of the optimum pH may be performed by conventional methods known to those of ordinary skill in the art. Suitable buffers may also be used to maintain the pH of the formulation.

The polypeptides of this invention may also be administered in the form of suppositories for rectal administration of the drug. These compositions can be prepared by mixing the drug with a suitable nonirritating excipient, which is solid at ordinary temperatures but liquid at the rectal temperatures and will therefore melt in the rectum to release the drug. Non-limiting examples of such materials are cocoa butter and polyethylene glycols.

Methods for the production of antibodies directed to a specific peptide or fragment thereof are well known by those of skill in the art. Antibodies can be obtained by injecting an animal with an immunogenic peptide of the invention, or an immunogenic fragment thereof, and recovering the antibodies which are able to complex with said immunogenic peptide or fragment thereof from said animal. Examples of such methods are disclosed in Antibodies, A Laboratory Manual, Harlow and Lane, Cold Spring Harbor Press, 1988, herein incorporated by reference.

Techniques which make it possible to humanize antibodies, either monoclonal or polyclonal, have been described in the following references: Waldmann T. (1991). Science. 252: 1657-1662; Winter G. et al. (1993). Immunology Today. 14(6): 243-246; Carter et al. (1992). Proc. Natl. Acad. Sci, USA. 89: 4285-4289; Singer et al. (1993). Journal of Immunology. 150(7): 2844-2857, Kipriyanov et al. (2004). Mol. Biotechnol. 26(1):39-60; U.S. Pat. No. 6,693,176; U.S. Pat. No. 5,225,539; U.S. Pat. No. 6,572,857; U.S. Pat. No. 6,183,744, each of which are incorporated herein by reference.

An ipaB mutant strain of S. flexneri expresssing IncA/cya was deposited at C.N.C.M., 25, Rue de Docteur Roux, F-75724, Paris Cedex 15, France, on Dec. 13, 2000, with accession number I-2592.

A bacterial strain containing the vector pUC19cya was deposited at C.N.C.M., 25, Rue de Docteur Roux, F-75724, Paris Cedex 15, France, on Dec. 13, 2000, with accession number I-2593.

An ipaB⁻ mutant strain of S. flexneri designated SF620 was deposited at C.N.C.M., 25, Rue de Docteur Roux, F-75724, Paris Cedex 15, France, on Dec. 13, 2000, with accession number I-2594.

A E. coli bacterial strain containing Psi0710 in an expression vector (pQE trisystem, Qiagen) with a carboxy-terminal Histidine tag was deposited at C.N.C.M., 25, Rue de Docteur Roux, F-75724, Paris Cedex 15, France, on Feb. 18, 2003, with accession number I-2974.

### EXAMPLES

Having generally described this invention, a further understanding can be obtained by reference to certain specific examples which are provided herein for purposes of illustration only, and are not intended to be limiting unless otherwise specified.

### Example 1

### Materials and Methods

Bacterial Strains and Reagents

Strain M90T is the virulent, wild-type strain of S. flexneri 5 (Sansonetti et al., 1982). Strains SF401 and SF620 (deposited at C.N.C.M. with accession number I-2594) are derivatives of M90T in which the mxiD and ipaB genes, respectively, have been inactivated (Allaoui, A., P. J. Sansonetti, and C. Parsot. (1993). MxiD, an outer membrane protein necessary for the secretion of the Shigella flexneri lpa invasins. Mol. Microbiol. 7: 59-68; Ménard, R., P. Sansonetti and C. Parsot. (1994). The secretion of the Shigella flexneri Ipa invasins is activated by epithelial cells and controlled by IpaB and IpaD. EMBO J 13: 5293-302). The E. coli strain TG1 (Sambrook et al., Molecular Cloning: A Laboratory Manual, Cold Spring Harbor, N.Y., 1989) was used for plasmid constructions. S. flexneri and E. coli strains were grown in Luria-Bertani (LB). Ampicillin was used at 0.1 mg/ml. Monoclonal antibodies against the calmodulin-dependent adenylate cyclase (Brézin et al, 1987). Evidence for the presence of cAMP, cAMP receptor and transcription termination factor Rho in different gram-negative bacteria. J. Gen. Microbiol. 131(11): 2953-2960) were used. Anti-IpaD antibody was used as described (Ménard, R., P. J. Sansonetti, and C. Parsot. (1993). Horseradish peroxidase-linked secondary antibodies for enhanced chemiluminescence were obtained from Amersham Pharmacia Biotech (Orsay, France). Alkaline phosphatase-linked secondary antibodies for enhanced chemifluorescence were obtained from Pierce (Rockford, Ill., USA).

Genomic Data

C. pneumoniae protein sequences (CPnXXXX), obtained by sequencing of the CWL029 strain of C. pneumoniae, are available at http://chlamydia-www.berkeley.edu:4231/ (chromosome sequence Genbank Accession number: AE001363), see also Kalman et al. (1999) Nature Genetics. 21: 385-389. C. trachomatis protein sequences (CTXXX), obtained by sequencing serovar D (D/UW-3/Cx) trachoma biovar of C. trachomatis, are available at http://chlamydia-www.berkeley.edu:4231/ (chromosome sequence Genbank Accession number: AE001273), see also Stephens et al. (1998) Science. 282: 754-759. Chlamydophila psittaci (GPIC strain) sequencing by The Institute for Genomic Research (TIGR, Rockville, Md., Etats-Unis) is not complete but preliminary sequence data was obtained from The Institute for Genomic Research website at http://www.tigr.org. The present Inventors compared C. pneumoniae proteins with the translation in the six reading frames of the available sequence using a comparison tool available at http://tigrblast.tigr.org/ufmg/. Herein, the closest protein match from C. psittaci to the blasted protein from C. pneumoniae (CPnXXXX) is referred to as PsiXXXX.

The entire DNA and amino acid sequences of claimed CPnXXXX, CTXXX, and PsiXXXX designators are attached herewith. The amino acid sequences of the respective proteins used to construct the chimeras are indicated by capital letters.

Construction of Recombinant Plasmids Expressing Hybrid Proteins

Genomic DNA from C. pneumoniae strain TW183 (Kuo et al. (1995) Clinical Microbiology Reviews. 8(4):451-461), serovar D (D/UW-3/Cx) trachoma biovar of C. trachomatis and Chlamydophila psittaci GPIC strain (American Type Culture Collection, Virginia, U.S.A.; ATCC No. VR-2282), was prepared from purified bacteria using the RapidPrep Micro Genomic DNA isolation kit (Amersham Pharmacia Biotech). The C. pneumoniae DNA fragments used in the examples were amplified by PCR using the strain TW183 as a template. These fragments were sequenced and shown to be more than 99% identical to the sequences of the CWL029 strain. The 5′ part of different chlamydial genes, including about 30 nucleotides located upstream from the proposed translation start sites and the first 20 to 99 codons, were amplified by PCR using the primers listed in Table 1. For the Inc/cya constructs, the forward and reverse primers contained additional HindIII and XbaI sites, respectively, to allow cloning of the PCR fragments between the HindIII and XbaI sites of the puc19cya vector. This vector was constructed by cloning the XbaI-EcoRI fragment of plasmid pMS109, which carried the cya gene of Bordetella pertussis (Sory, M. P., and G. R. Cornelis. (1994), between XbaI and EcoRI sites of the pUC19 vector. In the recombinant plasmids, transcription of the hybrid genes was under the control of the lac promoter of the vector. Recombinant plasmids were amplified in E. coli TG1 and sequencing checked the sequence of all constructs. Methods of sequencing nucleic acids are known in the art and are described in, for example, Sambrook et al., Molecular Cloning: A Laboratory Manual, Cold Spring Harbor Laboratory, N.Y. (1989) and Current Protocols in Molecular Biology, Ausebel et al, eds., John Wiley and Sons, Inc., New York (2000).

TABLE I
Oligonucleotides used to construct Inc/cya chimeras.

| | | |
|---|---|---|
| Protein | Forward primer | Reverse primer |

| | | |
|---|---|---|
| CPn0099 | AGTCAAGCTTATGATTGTACGGACATAGAGACCG | AGTCTCTAGATGCTTTTCGGACCATAGTC |
| | (SEQ ID NO: 133) | (SEQ ID NO: 250) |
| CPn0012 | AGTCAAGCTTGTAGGTTTATTAAAGGGGATGTACC | AGTCTCTAGAATCCTCTTCCCAAGGAATCAG |
| | (SEQ ID NO: 134) | (SEQ ID NO: 251) |
| CPn0026 | AGTCAAGCTTGTAGCTGTTCTTTTCAGAGAGCTT | AGTCTCTAGATCGAATCGATTTGGAAGGAG |
| | (SEQ ID NO: 135) | (SEQ ID NO: 252) |
| CPn0028 | AGTCAAGCTTTACTTTTTGAAGGCTAGTACGTT | AGTCTCTAGATTCAATAATGCCAGAGCTTTTTC |
| | (SEQ ID NO: 136) | (SEQ ID NO: 253) |
| CPn0049 | AGTCAAGCTTGGAAGGAATATCGTTTACCTGCT | AGTCTCTAGATTCATCCACCCAAATAGCAC |
| | (SEQ ID NO: 137) | (SEQ ID NO: 254) |
| CPn0063 | AGTCAAGCTTGTAGATGCCCATCCTACCAACAG | AGTCTCTAGAAGTAAGAGGGAGCCCAGGA |
| | (SEQ ID NO: 138) | (SEQ ID NO: 255) |
| CPn0066 | AGTCAAGCTTTCCCTAAAATGAATAAACTAAGGA | AGTCTCTAGATGACGGGGGAGGTGTATTAG |
| | (SEQ ID NO: 139) | (SEQ ID NO: 256) |
| CPn0067 | AGTCAAGCTTAAGTGTCATTGAAAAGGTTCAGG | AGTCTCTAGATAGGAGGCCTTTCGATTGTTT |
| | (SEQ ID NO: 140) | (SEQ ID NO: 257) |
| CPn0104 (SEQ ID NO: 2) | AGTCAAGCTTGCCAACCAACGGTTAGACGAAA | AGCTTCTAGATGGAAAGGATACGGATTGG |
| | (SEQ ID NO: 141) | (SEQ ID NO: 258) |
| CPn0105 | AGTCAAGCTTGCGTGCCTTTTAGAAAATTTAGCA | AGTCTCTAGAGAATGGGGGAATACAAATCA |
| | (SEQ ID NO: 142) | (SEQ ID NO: 259) |
| CPn0130 | AGTCAAGCTTGTAACGACTCCTTCTTTCAACGATT | AGTCTCTAGAGGCGAGAGAAAGTTFCGTTA |
| | (SEQ ID NO: 143) | (SEQ ID NO: 260) |
| CPn0132 | AGTCAAGCTTCCATTAAGGATCTAAAACAATTT | AGTCTCTAGACAACTTGTTCATGCGACAG |
| | (SEQ ID NO: 144) | (SEQ ID NO: 261) |
| CPn0146 | AGTCAAGCTTTGTTTGAGATGAATTCGCATTTT | AGCTTCTAGACGCATCCGAAAGACTTTTCT |
| | (SEQ ID NO: 145) | (SEQ ID NO: 262) |
| CPn0167 | AGTCAAGCTTTTGCATGAATTCGTAAAATAGAAA | AGTCTCTAGAACGTTCTAAGCCGTAATTCTCG |
| | (SEQ ID NO: 146) | (SEQ ID NO: 263) |
| CPn0174 | AGTCAAGCTTGTAGGGTTTGTGGAGAAAATTGTTA | AGTCTCTAGATGCGGGGATCGTATAAGCTA |
| | (SEQ ID NO: 147) | (SEQ ID NO: 264) |
| CPn0175 | AGTCAAGCTTCTTCTATTAGCCTGTTTCCAATT | AGTCTCTAGAAGGTGATTGCTTCCCAAGTCT |
| | (SEQ ID NO: 148) | (SEQ ID NO: 265) |
| CPn0181 | AGTCAAGCTTCTGTTGTTGAATTAATAGCTTCTT | AGTCTCTAGAATAAGAGTCGAGGGCTTGCAC |
| | (SEQ ID NO: 149) | (SEQ ID NO: 266) |
| CPn0186 | AGTCAAGCTTGTGAGAAAAACAACAATTCTTATCC | AGTCTCTAGATGTAGGAATACCTGGAGTCGTG |
| | (SEQ ID NO: 150) | (SEQ ID NO: 267) |
| CPn0206 (SEQ ID NO: 4) | AGTCAAGCTTACGACTCAATTAACAGTGATGCAA | AGTCTCTAGAGCGCTGAGTAGCGTCGTGTAA |
| | (SEQ ID NO: 151) | (SEQ ID NO: 268) |
| CPn0210 (SEQ ID NO: 6) | AGTCAAGCTTTCAAGACAATTAGAGAGAAAGAGC | AGCTTCTAGAGAATTCAAGATGATCCAAACA |
| | (SEQ ID NO: 152) | (SEQ ID NO: 269) |
| CPn0211 | AGTCAAGCTTGTAGGCATTGCTTAAAAATAAAATG | AGTCTCTAGAAGTCTTATGAAGCAAAGCAG |
| | (SEQ ID NO: 153) | (SEQ ID NO: 270) |
| CPn0220 | AGTCAAGCTTGTAATCGGATTTTAAACCAACTTTT | AGTCTCTAGAATTTATATGCCACGCCTTCTTTC |
| | (SEQ ID NO: 154) | (SEQ ID NO: 271) |
| CPn0223 | AGTCAAGCTTGTAGGAATTTTTATTACGAGTTTCA | AGTCTCTAGAGGAGGTTTCCGAGACGATT |
| | (SEQ ID NO: 155) | (SEQ ID NO: 272) |
| CPn0226 | AGTCAAGCTTCAAGAAGCGTTAAGAAAACGAAA | AGTCTCTAGAATCCCAGTCGTGAGAAGCAT |
| | (SEQ ID NO: 156) | (SEQ ID NO: 273) |
| CPn0243 | AGTCAAGCTTTTATTAAAATTTGTTAAAGGGAGG | AGTCTCTAGAGAGAAGAATATCTGCAACTAGAGC |
| | (SEQ ID NO: 157) | (SEQ ID NO: 274) |
| CPn0267 | AGTCAAGCTTGTAAACTAATTCGGATTAAAAATGA | AGTCTCTAGAGGTAACAGTGCATGCGAAAG |
| | (SEQ ID NO: 158) | (SEQ ID NO: 275) |
| CPn0277 | AGTCAAGCTTGAAGGAAAAGAAGTTCTCATAAAG | AGTCTCTAGACAAAATAGGAATAGCAGCTTGG |
| | (SEQ ID NO: 159) | (SEQ ID NO: 276) |
| CPn0284 | AGTCAAGCTTGTAGCGTCTGGAAAAATTCTGG | AGTCTCTAGACGGTCTTAAACTACTTTTCAATG |
| | (SEQ ID NO: 160) | (SEQ ID NO: 277) |
| CPn0287 | AGTCAAGCTTGGGTAAGAACACCTTCTAATATG | AGTCTCTAGAATCAACCACATCTTCTGGACAA |
| | (SEQ ID NO: 161) | (SEQ ID NO: 278) |
| CPn0291 | GACTAAGCTTGTAATCTATTTTTAGATAGGAA | GACTTCTAGATCCAGGTTTTTCGGAAGCAGA |
| | (SEQ ID NO: 162) | (SEQ ID NO: 279) |
| CPn0292 | AGTCAAGGTTCCCGTAATTACTGTCCGTACAAC | AGTCTCTAGAAGTAGACGGGAGTTGCTG |
| | (SEQ ID NO: 163) | (SEQ ID NO: 280) |
| CPn0308 | GACTAAGCTTATTATATAGACAGATTAAAAT | GACTTCTAGACTTAAAAAATACCCAGGAACA |
| | (SEQ ID NO: 164) | (SEQ ID NO: 281) |
| CPn0330 | AGTCAAGCTTAGGGTTTTAAGTCGGTTTGATG | AGTCTCTAGACCGAATCGCTTCATTCGTAG |
| | (SEQ ID NO: 165) | (SEQ ID NO: 282) |
| CPn0334 | AGTCAAGCTTTCGATAATCATAATTCAAAGCGTA | AGTCTCTAGAAAGGATCAGATGTGCTTTAGGG |
| | (SEQ ID NO: 166) | (SEQ ID NO: 283) |
| CPn0357 | AGTCAAGCTTTCCATGAATTCTAAAATGATTAGG | AGTCTCTAGACGAAAATTGGGTAGCCTGAC |
| | (SEQ ID NO: 167) | (SEQ ID NO: 284) |
| CPn0365 | AGTCAAGCTTAATTTAAAGGAACTCAGGTGAA | AGTCTCTAGAATCAAAGTTGCTGCAGGATT |
| | (SEQ ID NO: 168) | (SEQ ID NO: 285) |
| CPn0374 | AGTCAAGCTTGTAACATGGGGTATGGAAAGACC | AGTCTCTAGACTCGGCATCCTTTTGTTTTG |
| | (SEQ ID NO: 169) | (SEQ ID NO: 286) |
| CPn0379 | AGTCAAGCTTGTAAGCACCTGCTCTCGAATACA | AGTCTCTAGATTCTTCTTGGTGCCTGCTAA |
| | (SEQ ID NO: 170) | (SEQ ID NO: 287) |
| CPn0399 (SEQ ID NO: 8) | AGTCAAGCTTGTAACGGTCTCTTTGCTTGTTTTT | AGTCTCTAGAAGTGCAGCTGGATAGGGTTG |
| | (SEQ ID NO: 171) | (SEQ ID NO: 288) |
| CPn0405 (SEQ ID NO: 10) | AGTCAAGCTTGTAACGATCCGAACTTATCGTAGT | AGTCTCTAGATCCGGATGCGGTAGTAGTTG |
| | (SEQ ID NO: 172) | (SEQ ID NO: 289) |
| CPn0443 (SEQ ID NO: 12) | AGTCAAGCTTGTAAATTCATGGGCCTAATGATAA | AGTCTCTAGATAACCCTGTGGATGACGTTTT |
| | (SEQ ID NO: 173) | (SEQ ID NO: 290) |
| CPn0480 (SEQ ID NO: 14) | AGTCAAGCTTGTAATTGGGGAATCTCTGGGAGAC | AGTCTCTAGAGGGACTAACGACCTCAGCAC |
| | (SEQ ID NO: 174) | (SEQ ID NO: 291) |
| CPn0489 (SEQ ID NO: 16) | AGTCAAGCTTGTAATGGAGGATTGGCTAAGGA | AGTCTCTAGAAACACCACCGACATCACAAA |
| | (SEQ ID NO: 175) | (SEQ ID NO: 292) |
| CPn0490 (SEQ ID NO: 18) | AGTCAAGCTTGTAAGAGGGACCTTACCTATTGCTT | AGTCTCTAGAAATGAGGACCTCTCCTTCGT |
| | (SEQ ID NO: 176) | (SEQ ID NO: 293) |
| CPn0497 (SEQ ID NO: 20) | AGTCAAGCTTGCGGAATGGTTAAAGGAAACG | AGTCTCTAGATTGTCCATCAAAGCCTACAAT |
| | (SEQ ID NO: 177) | (SEQ ID NO: 294) |
| CPn0522 (SEQ ID NO: 22) | AGTCAAGCTTGTAAAACCAGCCTAGCCTTCAAA | AGTCTCTAGAGCTTTTTGCATAGGGGAAG |
| | (SEQ ID NO: 178) | (SEQ ID NO: 295) |
| CPn0556 (SEQ ID NO: 24) | AGTCAAGCTTGTAACCTGCTTCTTTAGGAACTAC | AGTCTCTAGATTGTAGACCAGCGGTGACACT |
| | (SEQ ID NO: 179) | (SEQ ID NO: 296) |
| CPn0582 (SEQ ID NO: 26) | AGTCAAGCTTGTAAAATTCAGCACAGGCTTGTAA | AGTCTCTAGAGCGTTCTGGAGTGACGAAAC |
| | (SEQ ID NO: 180) | (SEQ ID NO: 297) |
| CPn0585 | GACTAAGCTTGTAAATTGGAGATTGTAGTAGC | GACTTCTAGAAACAATTGTATGATTCCATCC |
| | (SEQ ID NO: 181) | (SEQ ID NO: 298) |
| CPn0588 (SEQ ID NO: 28) | ATGCAAGCTTACGAGCAAGAGCATAAAATCCA | ATGCTCTAGACCTTTGGCGGACTTTTTCTT |
| | (SEQ ID NO: 182) | (SEQ ID NO: 299) |
| CPn0595 (SEQ ID NO: 30) | AGTCAAGCTTGAATTGCTAACAGACACTAAAGG | AGTCTCTAGATACCCATTCTTGACCGGAAA |
| | (SEQ ID NO: 183) | (SEQ ID NO: 300) |
| CPn0648 | AGTCAAGCTTGTTTTAAAAAGCCTTGTAAGGAGGT | AGTCTCTAGACTGAGCAAAGGAGCTGACAG |
| | (SEQ ID NO: 184) | (SEQ ID NO: 301) |
| CPn0671 (SEQ ID NO: 32) | AGTCAAGCTTGTAAGAATTACCATAAATCAGAGGAA | AGTCTCTAGAAGCATCAGTGCAATGAGGATAA |
| | (SEQ ID NO: 185) | (SEQ ID NO: 302) |
| CPn0673 (SEQ ID NO: 34) | AGTCAAGCTTGTAACAGAAGGAAAAGCATACCACTG | AGTCTCTAGATCCAAAACGATCCTGTTCC |
| | (SEQ ID NO: 186) | (SEQ ID NO: 303) |
| CPn0681 (SEQ ID NO: 36) | AGTCAAGCTTGCCATAAGTCGTTTACAAGATCG | AGTCTCTAGATTCCACACAAGAGACCACCA |
| | (SEQ ID NO: 187) | (SEQ ID NO: 304) |
| CPn0705 | AGTCAAGCTTGTGAATCAGGGGGAAGCTAGT | AGTCTCTAGATCGTGTTTGCTTTCCTTCCA |
| | (SEQ ID NO: 188) | (SEQ ID NO: 305) |
| CPn0710 | AGTCAAGCTTCGCAAGATGATATAAAGGTCCA | AGTCTCTAGAGACAGTGCCTTGTGTTGATG |
| | (SEQ ID NO: 189) | (SEQ ID NO: 306) |
| CPn0711 | AGTCAAGCTTTAAATAACCAAGTATTGGGGTTTA | AGTCTCTAGATCGAAGCAGCGCATGTAACT |
| | (SEQ ID NO: 190) | (SEQ ID NO: 307) |
| CPn0712 (SEQ ID NO: 38) | AGTCAAGCTTGTAGACATGGCTGTCAGATCTTGG | AGTCTCTAGAAGAGTCGCGTCCTATAGACCA |
| | (SEQ ID NO: 191) | (SEQ ID NO: 308) |
| CPn0720 (SEQ ID NO: 40) | AGTCAAGCTTCTCTTTTAAAGACAACGCGAAT | AGCTTCTAGACGTGTGAGTCCCCTGAACTT |
| | (SEQ ID NO: 192) | (SEQ ID NO: 309) |
| CPn0725 (SEQ ID NO: 42) | AGTCAAGCTTGTAATTAATCTTGCGGAGATGTTGG | AGTCTCTAGACTCTGTAGTCAGCTGGTCGTTG |
| | (SEQ ID NO: 193) | (SEQ ID NO: 310) |
| CPn0729 (SEQ ID NO: 44) | AGTCAAGCTTGTAAGCCTGCTTGGTCTTCTGTT | AGTCTCTAGACGAGAGAGGAAGTTCAGCGTA |
| | (SEQ ID NO: 194) | (SEQ ID NO: 311) |
| CPn0746 (SEQ ID NO: 46) | ATGCAAGCTTGTAAGGGAAGATGTCCGTTCAGTT | ATGCTCTAGAAGCTCCCTTAACGACTTCTGG |
| | (SEQ ID NO: 195) | (SEQ ID NO: 312) |
| CPn0755 (SEQ ID NO: 48) | AGTCAAGCTTATGGCTATGAAGAGCAATTTATTC | AGTCTCTAGAAGGGTAACACACTAGGGGAAGA |
| | (SEQ ID NO: 196) | (SEQ ID NO: 313) |
| CPn0761 (SEQ ID NO: 50) | AGTCAAGCTTGTAACTCCCGCACCAACCTC | AGTCTCTAGATCCTTCAGACCAACGCTGATA |
| | (SEQ ID NO: 197) | (SEQ ID NO: 314) |
| CPn0764 (SEQ ID NO: 52) | AGTCAAGCTTGTAATCTGGTTAACACATGCTGAGG | AGTCTCTAGATGACAGGCCGTTTCTATCAA |
| | (SEQ ID NO: 198) | (SEQ ID NO: 315) |
| CPn0770 (SEQ ID NO: 54) | AGTCAAGCTTGTAAGAGCCGAGCATAGATAGAAAC | AGTCTCTAGAGCCTGCAATCAATCCCTGT |
| | (SEQ ID NO: 199) | (SEQ ID NO: 316) |
| CPn0774 (SEQ ID NO: 56) | AGTCAAGCTTTTAATAAGCTGAATAAACCAATGA | AGTCTCTAGATTCTGCGGAAGGAAGCTGT |
| | (SEQ ID NO: 200) | (SEQ ID NO: 317) |
| CPn0792 (SEQ ID NO: 58) | AGTCAAGCTTGTAACATGACGACATCACCTTATT | AGTCTCTAGAAGCGGCAGAAAATGAGAAAA |
| | (SEQ ID NO: 201) | (SEQ ID NO: 318) |
| CPn0820 | AGTCAAGCTTGTAAGTCAGGAAGTCCGTGGTGAT | AGTCTCTAGACAAAGCAATTAGAGTGAACGACA |
| | (SEQ ID NO: 202) | (SEQ ID NO: 319) |
| CPn0821 | AGTCAAGCTTGTAATTTGCGGTTGGGAAATAA | AGTCTCTAGAAGCACAGGCACAACGTTTAC |
| | (SEQ ID NO: 203) | (SEQ ID NO: 320) |
| CPn0829 | AGTCAAGCTTGTAACCCTTGCCTCTATTTTGAGA | AGTCTCTAGACGGTACCAAGGGCCATTTTG |
| | (SEQ ID NO: 204) | (SEQ ID NO: 321) |
| CPn0853 (SEQ ID NO: 60) | AGTCAAGCTTGTAATTGCGAGAAGGATTAAATCTTAG | AGTCTCTAGAAAGATGTCGGGAAAGTGTCG |
| | (SEQ ID NO: 205) | (SEQ ID NO: 322) |
| CPn0859 (SEQ ID NO: 62) | AGTCAAGCTTATCTCAAACATCAAGTGCTGAA | AGTCTCTAGATTGGCTTGCCTTATCATTCG |
| | (SEQ ID NO: 206) | (SEQ ID NO: 323) |
| CPn0879 (SEQ ID NO: 64) | AGTCAAGCTTGTAAGCCTCGTCAAATCCTGA | AGTCTCTAGAGACGACTTGCTTGCTCACT |
| | (SEQ ID NO: 207) | (SEQ ID NO: 324) |
| CPn0906 (SEQ ID NO: 66) | AGTCAAGCTTTCTTCAGGAACTTTAAAAACA | AGTCTCTAGATGCTGCGACACGAATTTCTA |
| | (SEQ ID NO: 208) | (SEQ ID NO: 325) |
| CPn0939 (SEQ ID NO: 68) | AGTCAAGCTTTAATAGAGCTCATTGGAGGAGGA | AGTCTCTAGAGAGGAGGGAAACACCGTTAAT |
| | (SEQ ID NO: 209) | (SEQ ID NO: 326) |
| CPn1002 (SEQ ID NO: 70) | AGTCAAGCTTGTAATGTAGAAAAGCGCAGAGAGAAA | AGTCTCTAGACCCTTCCAATTGAGGAAAA |
| | (SEQ ID NO: 210) | (SEQ ID NO: 327) |
| CPn1005 (SEQ ID NO: 72) | AGTCAAGCTTGTAATCGAAAACCAGTTAGGTTGAA | AGTCTCTAGATGCAATGATATCGTCAGCAG |
| | (SEQ ID NO: 211) | (SEQ ID NO: 328) |
| CPn1007 (SEQ ID NO: 74) | AGTCAAGCTTGTAATGGGTCCTAATGGAGGCTTT | AGTCTCTAGACACCTGGATTTTGCCTTCAG |
| | (SEQ ID NO: 212) | (SEQ ID NO: 329) |
| CPn1016 | AGTCAAGCTTGACCTCCTTGTAACCATTCTCG | AGTCTCTAGACTTCCATGGTAAAGGAGCA |
| | (SEQ ID NO: 213) | (SEQ ID NO: 330) |
| CPn1019 (SEQ ID NO: 76) | AGTCAAGCTTTCCTTAAGAGGATAAATCCACAG | AGTCTCTAGATTCTGCAGCAACAACAGCTT |
| | (SEQ ID NO: 214) | (SEQ ID NO: 331) |
| CPn1020 (SEQ ID NO: 78) | AGTCAAGCTTTTCGAAAACATAAATAATGTGGA | AGTCTCTAGAAGCTTCTTGAGGCGCTACAG |
| | (SEQ ID NO: 215) | (SEQ ID NO: 332) |
| CPn1022 | AGTCAAGCTTGTGCAGGAAAATGATGTTTTAGC | AGTCTCTAGATCCAGAGGCTGTGGAAACTGT |
| | (SEQ ID NO: 216) | (SEQ ID NO: 333) |
| CPn1027 | AGTCAAGCTTCAGCCTGGTTTCGTAATTTT | AGTCTCTAGAAGCATGAGGCTGTATGAGG |
| | (SEQ ID NO: 217) | (SEQ ID NO: 334) |
| CPn1032 (SEQ ID NO: 80) | AGTCAAGCTTGTAAACCACACCAATATTATTTAGGA | AGTCTCTAGATGCTTGTAGAAGAGCAGAATCG |
| | (SEQ ID NO: 218) | (SEQ ID NO: 335) |
| CPn1058 (SEQ ID NO: 82) | AGTGAAGCTTGTAACCTGGGCAGGTCAAAATCTA | AGTCTCTAGACCCAGATGAAGTCACTGGAAC |
| | (SEQ ID NO: 219) | (SEQ ID NO: 336) |
| CT053 | AGTCAAGCTTGTAATGGGTTTCCGGTTTCAATCA | AGTCTCTAGAACGGATTTCTTCCTGGTGTCT |
| | (SEQ ID NO: 220) | (SEQ ID NO: 337) |
| CT083 | AGTCAAGCTTGTAAGCCAAGAGCAGGATAAAACGA | AGTCTCTAGATGATATGCTCCCATTCTTTCG |
| | (SEQ ID NO: 221) | (SEQ ID NO: 338) |
| CT387 (SEQ ID NO: 84) | AGTCAAGCTTGTAAATTTTTCCTATAAGCTGGTTC | AGTCTCTAGATCCTTCGTATACGCCAGTACC |
| | (SEQ ID NO: 222) | (SEQ ID NO: 339) |
| CT476 (SEQ ID NO: 86) | AGTCAAGCTTTTGATAAGAAATTAGCCAACATAGG | AGTCTCTAGAGCATCCACGTTTGTTCCATT |
| | (SEQ ID NO: 223) | (SEQ ID NO: 340) |
| CT529 | AGTCAAGCTTTTCGGTTTAAGTAATAGAAGTGG | AGTCTCTAGAAGCATTCCCTGTACCAGACC |
| | (SEQ ID NO: 224) | (SEQ ID NO: 341) |
| CT550 (SEQ ID NO: 88) | AGTCAAGCTTGTAAGACGGTGCCTTCAATAACAAA | AGTCTCTAGAAGCCGAACGAGCTAAGACAT |
| | (SEQ ID NO: 225) | (SEQ ID NO: 342) |
| CT606.1 (SEQ ID NO: 90) | AGTCAAGCTTGTAAAAACAGGGAAAGACGATGA | AGTCTCTAGAGACCAAAGCTCCCTCTTGAA |
| | (SEQ ID NO: 226) | (SEQ ID NO: 343) |
| CT610 (SEQ ID NO: 92) | AGTCAAGCTTGTAAGCGGTTACGTGGAGTCAA | AGTCTCTAGAGGCATACGCCTGTAATTGCT |
| | (SEQ ID NO: 227) | (SEQ ID NO: 344) |
| CT642 (SEQ ID NO: 94) | AGTCAAGCTTGTAATCCGGAGCCTTTCTCCTAT | AGTCTCTAGATTCTTCAGGGCCAGCAA |
| | (SEQ ID NO: 228) | (SEQ ID NO: 345) |
| CT652.1 (SEQ ID NO: 96) | AGTCAAGCTTGTAAAGATGAATGATTCCAGAAAG | AGTCTCTAGAAGGAAAGCCTATCGCACACA |
| | (SEQ ID NO: 229) | (SEQ ID NO: 346) |
| CT664 (SEQ ID NO: 98) | AGTCAAGCTTGGACTAAGTAAAACGGAGCAGGA | AGTCTCTAGAAGACCAACTCGTCCCATTTTC |
| | (SEQ ID NO: 230) | (SEQ ID NO: 347) |
| CT665 | AGTCAAGCTTTTCGAGGTTTATTTAATTCTTCCA | AGTCTCTAGAGTGGGCTTTGGTTACATCGT |
| | (SEQ ID NO: 231) | (SEQ ID NO: 348) |
| CT666 | AGTCAAGCTTACGTATCAACCGTAAAATGGTG | AGTCTCTAGAAGTTCCTTGCGTGGATGTTT |
| | (SEQ ID NO: 232) | (SEQ ID NO: 349) |
| CT671 | AGTCAAGCTTGTAAGCAAAAACAACGGGAATC | AGTCTCTAGACATCCGATCTGCTTTCTCTTG |
| | (SEQ ID NO: 233) | (SEQ ID NO: 350) |
| CT718 (SEQ ID NO: 100) | AGTCAAGCTTGTAAATGAAGAGTGCTGTGTTAAAGT | AGTCTCTAGAATGGGAGAGAGCTTCTTGCT |
| | (SEQ ID NO: 234) | (SEQ ID NO: 351) |
| CT763 (SEQ ID NO: 102) | AGTCAAGCTTGTAACACAATCTTGGGCAAGAGAC | AGTCTCTAGAGATCTCCAGCTTAAGGGTTGC |
| | (SEQ ID NO: 235) | (SEQ ID NO: 352) |
| CT845 (SEQ ID NO: 104) | AGTCAAGCTTGTAAGAGAAGAGAAAGGCTAAGGATG | AGTCTCTAGAAGGGTTCTCCTCAAGTTCATC |
| | (SEQ ID NO: 236) | (SEQ ID NO: 353) |
| CT848 (SEQ ID NO: 106) | AGTCAAGCTTATAAACCCCTGTCTTAACCCA | AGTCTCTAGACGTITGCAGAGTTCGTTGTTC |
| | (SEQ ID NO: 237) | (SEQ ID NO: 354) |
| CT863 | AGTCAAGCTTTCCTTTCTAAAAGGCCTGGA | AGCTTCTAGACAGAACAACGTCTCCTACGC |
| | (SEQ ID NO: 238) | (SEQ ID NO: 355) |
| Psi0330 (SEQ ID NO: 108) | AGTCAAGCTTGCAAATCTAAGGAGTTAGGTTAGGG | AGCTTCTAGAAGCCAAACGCATAGCTTCAT |
| | (SEQ ID NO: 239) | (SEQ ID NO: 356) |
| Psi0379 (SEQ ID NO: 110) | AGTCAAGCTTGTAATACAGGATGGTGCCCACT | AGTCTCTAGATAGAAGTTGCAGGCGTTCCT |
| | (SEQ ID NO: 240) | (SEQ ID NO: 357) |
| Psi0595 (SEQ ID NO: 112) | AGTCAAGCTTGCGCAAATAAACGATACAA | AGTCTCTAGATCCGTAGTTGTTACGGAAGGtT |
| | (SEQ ID NO: 241) | (SEQ ID NO: 358) |
| Psi0648 (SEQ ID NO: 114) | AGTCAAGCTTCATTGTGTTATAATTGCAGGCTA | AGTCTCTAGATTTCTTGGCTCCCTGCATAG |
| | (SEQ ID NO: 242) | (SEQ ID NO: 359) |
| Psi0671 (SEQ ID NO: 116) | AGTCAAGCTTTGACGCCCCTTAAAATAAAGA | AGTCTCTAGACGCAGAATGGGATAGGACAT |
| | (SEQ ID NO: 243) | (SEQ ID NO: 360) |
| Psi0710 (SEQ ID NO: 120) | AGTCAAGCTTCCGCAAAATGGTTTAACAGA | AGTCTCTAGATTGCACACCTTGGACGTACT |
| | (SEQ ID NO: 244) | (SEQ ID NO: 361) |
| Psi0761 (SEQ ID NO: 122) | AGTCAAGCTTGTAAGCTTGGGAATCTACACATTTTT | AGTCTCTAGATTTCGTTAATTCTCCCTTAGACCA |
| | (SEQ ID NO: 245) | (SEQ ID NO: 362) |
| Psi0774 (SEQ ID NO: 124) | AGTCAAGCTTGTAAAAGCTGAATAAGCCCATGA | AGTCTCTAGAAAGTTTCTGTGTCGCTACGG |
| | (SEQ ID NO: 246) | (SEQ ID NO: 363) |
| Psi1002 (SEQ ID NO: 126) | AGTCAAGCTTGTAACCGTGAGGAGTCTGAACAA | AGTCTCTAGAAGAAAAATCCATGGGCTGTAA |
| | (SEQ ID NO: 247) | (SEQ ID NO: 364) |
| Psi1005 (SEQ ID NO: 128) | AGTCAAGCTTTTAATCGAAGAAAATAGGTAGGAAA | AGTCTCTAGATTCATCGGCTGTTGCTGTAT |
| | (SEQ ID NO: 248) | (SEQ ID NO: 365) |
| Psi1022 (SEQ ID NO: 130) | AGTCAAGCTTGTAACGCTTCTACATCCCCATAATT | AGTCTCTAGAGAATACGGAAAGCGCAACAT |
| | (SEQ ID NO: 249) | (SEQ ID NO: 366) |

Transformation of S. flexneri ipaB Strain and Test of Secretion Via the Type III Pathway

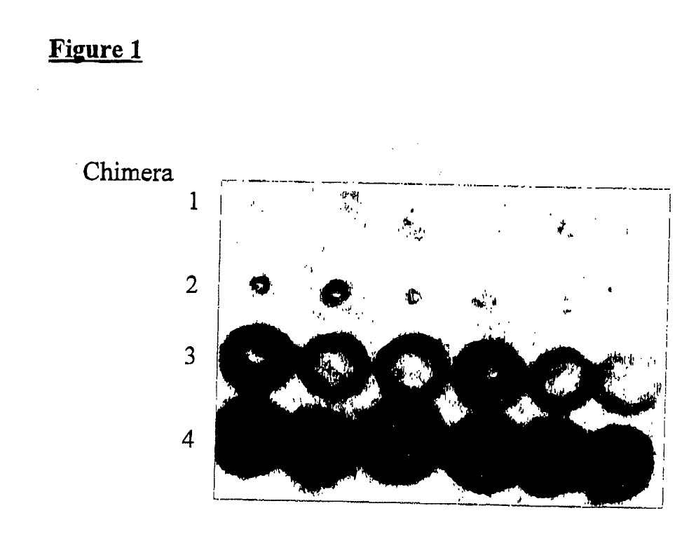

Shigella flexneri (ipaB strain, I-2594) were transformed with the constructs made by the process above and colonies expressing a CPn/cya, CT/cya, or Psi/cya chimera were isolated on plates containing the Congo Red dye. Only red colonies, indicative of a constitutive high level of type III secretion, were considered. Such colonies were picked on a LB plate and incubated for 6 hours at 37° C. A polyvinylidene fluoride membrane (Immobilon-P, Millipore) was deposited on top of the colonies, and the plate was incubated overnight at 37° C. The membrane was briefly incubated in ethanol and then processed for Western blotting using anti-cyclase antibody described (Subtil et al (2001) Mol. Microbiol. 39:792-800). Revelation was done by enhanced chemifluorescence (Amersham). The signal for colonies in which the CPn/cya, CT/cya, or Psi/cya chimeras were not secreted was restricted to the area of the membrane that had been in contact with the colonies. The signal for colonies in which the CPn/cya, CT/cya, or Psi/cya chimeras were secreted appeared as a halo around the area of the membrane that had been in contact with the colonies (see FIG. 1).

Negative Controls—

Chlamydial proteins that are homologous to cytosolic proteins in other bacteria species are not expected to be secreted during Chlamydia infection and therefore should not be positive for secretion in the present assay. To validate the specificity of this assay, the Inventors constructed 9 chimeras between putative cytosolic chlamydial proteins and the cya reporter molecule. None of these chimeras were secreted by the ipaB strain of S. flexneri. This result shows that the test developed by the present Inventors is specific for detecting secreted proteins.

The 9 chimeras that were constructed used the amino terminal region of CPn0032, CPn0089, CPn0103, CPn0115, CPn0184, CPn0202, CPn0280, CPn0320, CPn0402.

Negative Results—

Most of the 280 chimera that the Inventors tested were not secreted by the ipaB strain. This result was expected since in other bacteria species such as Shigella, type III secretion allows for the secretion of only a limited number of proteins (up to date about 25 have been identified for S. flexneri (Buchrieser C., P. Glaser, C. Rusniok, H. Nedjari, H. d'Hauteville, F. Kunst, P. Sansonetti, and C. Parsot. (2000). The virulence plasmid pWR100 and the repertoire of proteins secreted by the type III secretion apparatus of Shigella flexneri. Mol. Microbiol. 38(4): 760-771).

Examples of Chlamydial proteins for which the corresponding chimera were not secreted in the ipaB strain are:

CPn0010, CPn0011, CPn0034, CPn0036, CPn0042, CPn0046, CPn0050, CPn0051, CPn0055, CPn0062, CPn0070, CPn0071, CPn0084, CPn0085, CPn0087, CPn0089, CPn0093, CPn0095, CPn0099, CPn0100.

Detecting secreted proteins by the type III secretion system by Shigella flexneri, the secretion of chimeric forms (consisting of the amino-terminal domain of these proteins fused with the Cya reporter) of Chlamydia spp. proteins was determined. The secreted chimeric proteins can be classified in 5 categories:

Category 1—Corresponding chimeras from C. pneumoniae, C. trachomatis, and C. psittaci that are homologous and are secreted. Sequence analysis has shown that these three species are rather distant, and, as a consequence, the percentage of amino acid identity between homologous proteins is generally low. Consequently, the chimeras constructed from homologous proteins share little identity in their amino terminal region, which is the region containing the putative secretion signal. Therefore, the identification that the three homologous chimeras (one from each of the Chlamydia species above) are secreted strongly supports that the corresponding proteins are secreted during Chlamydia infection.

Proteins of C. pneumoniae (CPnXXXX), C. trachomatis (CTXXX), and C. psittaci (PsiXXXX) which are part of this category are:

CPn0330; CT083; Psi0330; CPn0379; CT053; Psi0379; CPn0595; CT476; Psi0595; CPn0648; CT529; Psi0648; CPn 0671; CT550; Psi0671; CPn0710; CT666; Psi0710; CPn0761; CT610; Psi0761; CPn0774; CT606.1; Psi0774; CPn1002; CT845; Psi1002; CPn1005; CT848; Psi1005; CPn1002; CT863; Psi1022.

Category 2—Proteins of C. pneumoniae and C. trachomatis which are homologous and for which the 2 corresponding chimeras are secreted. For the same reasons as explained above in the case of category 1, these results support that these proteins are likely secreted during Chlamydia infection.

Proteins of C. pneumoniae (CPnXXXX) and C. trachomatis (CTXXX) which are part of this category are:

CPn0490; CT387; CPn0705; CT671; CPn0711; CT665; CPn0712; CT664; CPn0725; CT652.1; CPn0770; CT642; CPn0859; CT718; CPn0906; CT763.

Category 3—C. pneumoniae proteins that are secreted, which have no homolog in C. trachomatis or C. psittaci. They may be important proteins, being specific of C. pneumoniae.

C. pneumoniae proteins (CPnXX) which are part of this category are:

CPn0009; CPn0012; CPn0028; CPn0049; CPn0063; CPn0066; CPn0067; CPn0130; CPn0132; CPn0146; CPn0167; CPn0174; CPn0175; CPn0181; CPn0210; CPn0211; CPn0220; CPn0223; CPn0226; CPn0243; CPn0267; CPn0277; CPn0284; CPn0357; CPn0365; CPn0585; CPn0829; CPn1027.

Category 4 —C. pneumoniae proteins that have a homolog in at least one of the other 2 sequenced Chlamydia species and for which secretion of the corresponding homologous chimera have not been tested for technical or scientific reason (for example when the percentage of identity between the homologous proteins was very high).

C. pneumoniae (CPnXXXX) proteins that have a homolog in at least one of the other 2 sequenced Chlamydia species and for which secretion of the corresponding homologous chimeras have not been tested, and are part of this category are:

CPn0026; CPn0104; CPn0186; CPn0206; CPn0291; CPn0292; CPn0405; CPn0443; CPn0480; CPn0489; CPn0556; CPn0673; CPn0681; CPn0720; CPn0746; CPn0853; CPn0879; CPn0939; CPn1019; CPn1020; CPn1032.

Category 5—C. pneumoniae proteins that have homologs in the other 2 sequenced Chlamydia species and for which secretion of the corresponding homologous chimeras have either not been tested or are negative or undetermined. Although these results support that most proteins in this category are secreted by Chlamydia during infection, the Inventors regard them as somewhat less strong candidates than the proteins classified in the other 4 categories, until more data are obtained (see for example the result with Psi1058 below).

C. pneumoniae (CPnXXXX) proteins that have homologs in the other 2 sequenced Chlamydia species and for which secretion of the corresponding homologous chimeras have either not been tested or are negative or undetermined, and are part of this category are:

CPn0105; CPn0287; CPn0334; CPn0374; CPn0399; CPn0497; CPn0522; CPn0582; CPn0588; CPn0729; CPn0755; CPn0764; CPn0792; CPn0820; CPn0821; CPn1007; CPn1016; CPn1058.

### Example 2

Chlamydial genes were cloned by PCR for expression of full-length chlamydial proteins with a carboxy-terminal Histidine-tag. The forward and reverse primers contained additional NcoI and KpnI sites, respectively, to allow cloning of the PCR fragments between the NcoI and KpnI sites of the pQE-TriSystem expression vector (Qiagen). Sequences of the primers are given in Table II. Recombinant plasmids were amplified in E. coli TG1 and the sequences were checked by sequencing.

Plasmids were used to transform the strains SF401 and SF620 which are derivatives of M90T in which the mxiD and ipaB genes, respectively, have been inactivated (Allaoui et al., 1993; Ménard et al., 1993). Transformed colonies were isolated on plates containing 100 μg/ml ampicillin.

Analysis of secreted proteins was performed as previously described (Allaoui et al., 1993). Briefly, 1 ml of an overnight culture at 30° C. was inoculated in 30 ml of LB and incubated at 37° C. for 3 h. Bacteria were harvested by centrifugation and the culture supernatant was filtered through 0.22 μm filters. Proteins present in 25 ml of the filtrates were precipitated by the addition of 1/10 (v/v) trichloroacetic acid, and resuspended in 0.5 ml of sample buffer for electrophoresis. Equal volumes of samples of the bacterial pellet and culture supernatants, the latter being concentrated 25-fold as compared to the pellet, were analyzed by electrophoresis in 12% polyacrylamide gels in the presence of sodium dodecyl sulfate (SDS-PAGE). After electrophoresis, proteins were transferred on a PVDF membrane (Millipore Corporation, Bedford, Mass.), and the membrane was used for blotting with anti-His antibody (H-15, sc-803, Santa Cruz Biotechnology). Western blotting and revelation were performed by enhanced chemiluminescence (Amersham Pharmacia Biotech) according to the manufacturer's instructions.

TABLE II

| | | |
|---|---|---|
| Protein | Forward primer | Reverse primer |

| | | |
|---|---|---|
| CPn0175 | ATCGTACCATGGAGCAACCCAATTGTG | AGTCGGTACCAGCTTCCTTAACTTCGCTGAG |
| | (SEQ ID NO: 367) | (SEQ ID NO: 373) |
| Psi0705 | ATCGTACCATGGAATTAAATAAAACATCCGAGTCTTTG | AGTCGGTACCTTGGTTTTGTTCTTGATCTTGC |
| | (SEQ ID NO: 368) | (SEQ ID NO: 374) |
| Psi0725 | ATCGTACCATGGTTCTCGCTTCTTGTCTTCTTG | AGTCGGTACCATCTAAGAAATCATCTTCTGTAAGGAC |
| | (SEQ ID NO: 369) | (SEQ ID NO: 375) |
| Psi0774 | ATCGTACCATGGATGAAGGTATTCAAACCGTTTCT | AGTCGGTACCGTCTCTAGGAACTAGCGTTTCCA |
| | (SEQ ID NO: 370) | (SEQ ID NO: 376) |
| Psi1005 | ATCGTACCATGGGGTTCTCTTCTCCAGCACTTC | AGTCGGTACCAATGTTTGCTATCAAACTTACAATT |
| | (SEQ ID NO: 371) | (SEQ ID NO: 377) |
| Psi1058 | ATCGTACCATGGAGAACAAAACACTAAGCGTTTTCT | AGTCGGTACCCAATGAAAATAGAGGCGGA |
| | (SEQ ID NO: 372) | (SEQ ID NO: 378) |

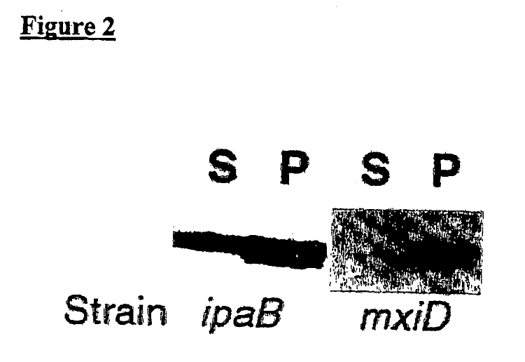

The present inventors have shown that CPn0175, Psi0705, Psi0725, Psi0774, Psi1005 and Psi1058, expressed as full-length proteins with a C-terminal histidine tag, are found in the supernatant of the transformed ipaB cultures. When they are expressed in the mxiD strain, which is deficient for type III secretion, these proteins are absent from the culture supernatants (see FIG. 2). These results confirm that CPn0175, Psi0705, Psi0725, Psi0774, Psi1005 and Psi1058 are secreted by a type III mechanism in S. flexneri.

### Example 3

Proteins Psi0705 and Psi0710 were expressed in E. coli with a carboxy-terminal Histidine tag and purified by standard divalent metal column chromatography methods according to the manufacturer's instructions (Qiagen). Purified proteins were used to immunize rabbits and specific antibodies against Psi0705 and Psi0710 were obtained. These antibodies were used to study Psi0705 and Psi0710 localization during Chlamydia infection.

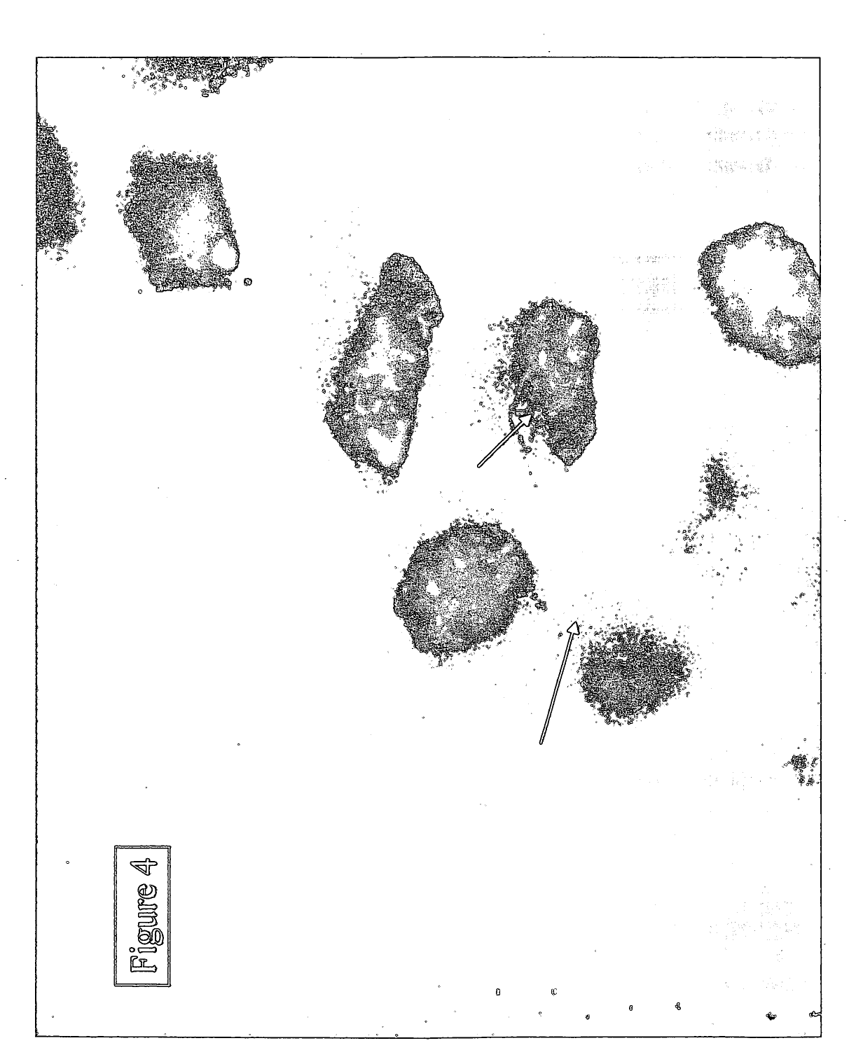

A: By immunofluorescence, the present inventors determined that Psi0710 is associated with the membrane of the inclusion in HeLa cells infected with C. psittaci GPIC strain (FIG. 4). As a control, the present inventors determined that the pre-immune serum did not label infected cells, and that antibodies against the Major Outer Membrane Protein of Chlamydia did not label the membrane of the inclusion.

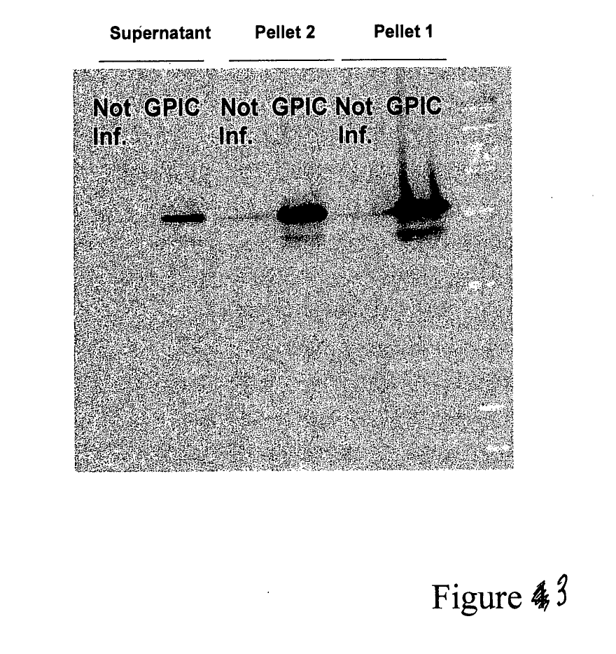

B: By Western Blot, the present inventors determined that Psi0705 is associated with the cytosolic fraction of HeLa cells infected with C. psittaci GPIC strain (FIG. 3). The same result was obtained using antibodies specific for Psi0710. As a control, the present inventors determined that the Major Outer Membrane Protein of Chlamydia was not found in the cytosolic fraction of HeLa cells infected with C. psittaci GPIC strain.

These experiments demonstrate that Psi0705 and Psi0710 are secreted by Chlamydia during infection. Therefore, these data fully confirm the inventive approach to identify such proteins using heterologous type III secretion as described in the present invention.

Obviously, numerous modifications and variations on the present invention are possible in light of the above teachings. It is therefore to be understood that within the scope of the appended claims, the invention may be practiced otherwise than as specifically described herein.

1. A purified secreted Chlamydia polypeptide, wherein said Chlamydia polypeptide is homologous to one or more Chlamydia pneumoniae proteins selected from the group consisting of CPn0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn0939, CPn1002, CPn1005, CPn1007, CPn1019, CPn1020, CPn1032, and CPn1058; or a fragment thereof.

2. The polypeptide according to claim 1, wherein said Chlamydia polypeptide is one or more Chlamydia pneumoniae proteins selected from the group consisting of CPn 0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn0939, CPn1002, CPn1005, CPn1007, CPn1019, CPn1020, CPn1032, and CPn1058; or a fragment thereof.

3. The polypeptide according to claim 1, wherein said Chlamydia polypeptide is one or more Chlamydia pneumoniae proteins selected from the group consisting of CPn0490, CPn0595, CPn0671, CPn0712, CPn0725, CPn0761, CPn0770, CPn0774, CPn0859, CPn0906, CPn1002, and CPn1005, or a fragment thereof, or wherein said Chlamydia polypeptide is homologous to one or more Chlamydia pneumoniae proteins selected from the group consisting of CPn0490, CPn0595, CPn0671, CPn0712, CPn0725, CPn0761, CPn0770, CPn0774, CPn0859, CPn0906, CPn1002, and CPn1005, or a fragment thereof.

4. The polypeptide according to claim 1, wherein the homologous Chlamydia polypeptide is a Chlamydia pneumoniae protein.

5. The polypeptide according to claim 1, wherein the homologous Chlamydia polypeptide is a Chlamydia trachomatis protein selected from the group consisting of CT387, CT476, CT550, CT606.1, CT610, CT642, CT652.1, CT664, CT718, CT763, CT845, and CT848; or a fragment thereof.

6. The polypeptide according to claim 1, wherein the homologous Chlamydia polypeptide is a Chlamydia psittaci protein selected from the group consisting of Psi0330, Psi0379, Psi0595, Psi0648, Psi0671, Psi0705, Psi0710, Psi0761, Psi0774, Psi1002 2, Psi1005, Psi1022, and Psi1058; or a fragment thereof.

7. The polypeptide according to claim 1, wherein said polypeptide is identified by its expression by a Gram-negative bacterial strain and secretion by the type III secretion pathway of said bacterial strain.

8. The polypeptide according to claim 1, wherein said Gram-negative strain containing a type III secretion pathway is a Shigella strain.

9. The polypeptide according to claim 1, wherein said polypeptide is selected by a method for identifying polypeptides secreted by Chlamydia comprising (a) providing a recombinant expression vector containing at least a polynucleotide coding for the polypeptide of interest; (b) transforming a Gram-negative strain containing a type III secretion pathway with said recombinant vector; (c) expressing this vector in the Gram-negative strain transformed in (b); and (d) detecting the secretion of said polynucleotide expression product; wherein the secretion of said expression product indicates that it corresponds to a secreted Chlamydia polypeptide.

10. The polypeptide according to claim 9, wherein said polypeptide is from Chlamydia pneumoniae.

11. The polypeptide according to claim 9, wherein said Gram-negative strain containing a type III secretion pathway is a Shigella strain.

12. The polypeptide according to claim 1, wherein said polypeptide is selected by a method for identifying polypeptides secreted by Chlamydia comprising (a) providing a recombinant expression vector comprising at least the DNA coding for the polypeptide of interest fused to a reporter gene; (b) transforming a Gram-negative strain containing a type III secretion pathway with said recombinant vector; (c) expressing this vector in the Gram-negative strain transformed in (b); and (d) detecting the secretion of said reporter gene expression product; wherein the secretion of said expression product indicates that the fused DNA contains at least a polynucleotide corresponding to a secreted Chlamydia polypeptide.

13. The polypeptide according to claim 12, wherein said polypeptide is from Chlamydia pneumoniae.

14. The polypeptide according to claim 12, wherein said Gram-negative strain containing a type III secretion pathway is a Shigella strain.

15. A purified polynucleotide encoding at least one polypeptide according to claim 1 or a purified polynucleotide which hybridizes to said purified polynucleotide encoding at least one polypeptide according to claim 1 under stringent conditions.

16. The purified polynucleotide according to claim 15, wherein said polynucleotide is a primer or a probe.

17. An immunogenic composition comprising at least a polypeptide according to claim 1 or an immunogenic fragment thereof.

18. A vaccinating composition against Chlamydia infection wherein said composition comprises at least one polypeptide according to claim 1 or an immunogenic fragment thereof along with a pharmaceutically acceptable carrier.

19. The vaccinating composition according to claim 18, wherein said polypeptide is from Chlamydia pneumoniae.

20. The vaccinating composition according to claim 18, wherein said infection contributes to atherosclerosis.

21. The vaccinating composition according to claim 18, wherein said infection is a sexually transmitted disease.

22. The vaccinating composition according to claim 18, wherein said infection is a respiratory disease.

23. The vaccinating composition according to claim 18, wherein said respiratory disease is bronchitis.

24. A complex comprising the polypeptide of claim 1 and an antibody directed against said polypeptide.

25. A complex comprising the polypeptide of claim 2 and an antibody directed against said polypeptide.

26. An antibody against Chlamydia wherein said antibody is directed against the polypeptide according to claim 1 or an immunogenic fragment thereof.

27. The antibody according to claim 26, wherein said polypeptide is from Chlamydia pneumoniae.

28. A method for diagnosing a Chlamydia infection in an animal wherein said method comprises: (a) providing an animal sample of a tissue suspected to be infected by Chlamydia; (b) bringing said sample into contact with an antibody according to claim 24;
and (c) detecting antigen-antibody complexion; wherein said complexion is indicative of a Chlamydia infection in said animal.

29. The method according to claim 28, wherein said Chlamydia infection is a Chlamydia pneumoniae infection.

30. A method of preventing or treating a Chlamydia infection in an animal, which comprises administering an effective amount of an antibody according to claim 26, or an immunogenic fragment thereof, to an animal in need thereof.

31. The method according to claim 30, wherein said Chlamydia infection is a Chlamydia pneumoniae infection.

32. A method for diagnosing a Chlamydia infection in an animal wherein said method comprises (a) providing a polypeptide according to claim 1, or an immunogenic fragment thereof, optionally labeled; (b) bringing said polypeptide or immunogenic fragment thereof into contact with a serum sample of said animal; and (c) detecting complexes formed between said polypeptide or immunogenic fragment thereof and antibodies contained in the serum sample; wherein said complexes are indicative of a Chlamydia infection in said animal.

33. The method according to claim 32, wherein said Chlamydia infection is a Chlamydia pneumoniae infection.

34. A method of detecting Chlamydia in an animal wherein said method comprises (a) providing an animal sample of a tissue suspected to be infected by Chlamydia; (b) adding a primer pair for a polypeptide according to claim 1 to the tissue sample; (c) amplifying a polynucleotide that encodes for the polypeptide which corresponds to the primer pair selected; and (d) detecting the presence of Chlamydia by the presence or absence of said polynucleotide.

35. A method of screening for an active molecule inhibiting the secretion of a secreted Chlamydia polypeptide, comprising (a) supplying an active molecule to a culture of Chlamydia; (b) growing or incubating said culture for a time and under conditions suitable for said active molecule to exert an activity upon said culture; (c) adding a primer pair for a polypeptide according to claim 1 to the culture; (d) amplifying a polynucleotide that encodes for the polypeptide which corresponds to the primer pair selected; and (e) detecting the presence of the secreted Chlamydia polypeptide by the presence or absence of said polynucleotide.

36. A plasmid for expression of secreted Chlamydia polypeptide wherein said plasmid contains at least a polynucleotide coding for a polypeptide according to claim 1.

37. The plasmid according to claim 36, wherein said polypeptide is from Chlamydia pneumoniae.

38. The plasmid according to claim 36, wherein said polynucleotide is further fused to a reporter gene.

39. The plasmid according to claim 38, wherein a vector deposited at C.N.C.M. on Dec. 13, 2000 with accession No. I-2593 is used for the construction of said plasmid.

40. A recombinant Gram-negative strain, wherein said strain is transformed by the plasmid according to claim 36.

41. The recombinant Gram-negative strain according to claim 40, wherein said strain is a Shigella strain.

42. A method of preventing or treating a Chlamydia infection in an animal which comprises administering an effective amount of a purified secreted Chlamydia polypeptide, wherein said Chlamydia polypeptide is homologous to one or more Chlamydia pneumoniae proteins selected from the group consisting of CPn0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn0939, CPn1002, CPn1005, CPn1007, CPn1019, CPn1020, CPn1032, and CPn1058; or a fragment thereof.

43. The method according to claim 42, wherein said Chlamydia polypeptide is one or more Chlamydia pneumoniae proteins selected from the group consisting of CPn0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn9939, CPn1002, CPn1005, CPn1007, CPn1019, CPn1020, CPn1032, and CPn1058; or a fragment thereof.

44. The method according to claim 42, wherein said Chlamydia infection is a Chlamydia pneumoniae infection.

45. The method according to claim 42, wherein said Chlamydia polypeptide is identified by its secretion in a Gram-negative bacterial strain containing a type III secretion pathway.

46. The method according to claim 42, wherein said animal is selected from the group consisting of a human, an equine, a bovine, a porcine, a caprine, a ovine, a bird, a dog, and a cat.

47. The method according to claim 42, wherein said animal is a human.

48. A purified secreted Chlamydia polypeptide obtainable by a type III secretion pathway, wherein said Chlamydia polypeptide is one or more Chlamydia pneumoniae proteins selected from the group consisting of CPn0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn0939, CPn1002, CPn1005, CPn1007, CPn1019, CPn1020, CPn1032, and CPn1058; or a fragment thereof; or a polypeptide recognized by an antibody raised against one or more Chlamydia pneumoniae proteins selected from the group consisting of CPn0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn0939, CPn1002, CPn1005, CPn1007, CPn1019, CPn1020, CPn1032, and CPn1058; or a fragment thereof.

49. A purified secreted Chlamydia polypeptide, wherein said Chlamydia polypeptide is homologous to one or more Chlamydia trachomatis proteins selected from the group consisting of CT387, CT476, CT550, CT606.1, CT610, CT642, CT652.1, CT664, CT718, CT763, CT845, and CT848; or a fragment thereof.

50. The polypeptide according to claim 49, wherein the homologous Chlamydia polypeptide is a Chlamydia trachomatis protein.

51. The polypeptide according to claim 49, wherein said polypeptide is identified by its expression by a Gram-negative bacterial strain and secretion by the type III secretion pathway of said bacterial strain.

52. The polypeptide according to claim 49, wherein said Chlamydia polypeptide is one or more Chlamydia trachomatis proteins selected from the group consisting of CT387, CT476, CT550, CT606.1, CT610, CT642, CT652.1, CT664, CT718, CT763, CT845, and CT848; or a fragment thereof.

53. The polypeptide according to claim 49, wherein said Gram-negative strain containing a type III secretion pathway is a Shigella strain.

54. The polypeptide according to claim 49, wherein said polypeptide is selected by a method for identifying polypeptides secreted by Chlamydia trachomatis comprising (a) providing a recombinant expression vector containing at least a polynucleotide coding for the polypeptide of interest; (b) transforming a Gram-negative strain containing a type III secretion pathway with said recombinant vector; (c) expressing this vector in the Gram-negative strain transformed in (b); and (d) detecting the secretion of said polynucleotide expression product; wherein the secretion of said expression product indicates that it corresponds to a secreted Chlamydia trachomatis polypeptide.

55. The polypeptide according to claim 54, wherein said Gram-negative strain containing a type III secretion pathway is a Shigella strain.

56. The polypeptide according to claim 49, wherein said polypeptide is selected by a method for identifying polypeptides secreted by Chlamydia trachomatis comprising (a) providing a recombinant expression vector comprising at least the DNA coding for the polypeptide of interest fused to a reporter gene; (b) transforming a Gram-negative strain containing a type III secretion pathway with said recombinant vector; (c) expressing this vector in the Gram-negative strain transformed in (b); and (d) detecting the secretion of said reporter gene expression product; wherein the secretion of said expression product indicates that the fused DNA contains at least a polynucleotide corresponding to a secreted Chlamydia trachomatis polypeptide.

57. The polypeptide according to claim 56, wherein said Gram-negative strain containing a type III secretion pathway is a Shigella strain.

58. A purified polynucleotide encoding at least one polypeptide according to claim 49 or a purified polynucleotide which hybridizes to said purified polynucleotide encoding at least one polypeptide according to claim 49 under stringent conditions.

59. The purified polynucleotide according to claim 58, wherein said polynucleotide is a primer or a probe.

60. An immunogenic composition comprising at least a polypeptide according to claim 49 or an immunogenic fragment thereof.

61. A vaccinating composition against Chlamydia trachomatis infection wherein said composition comprises at least one polypeptide according to claim 49 or an immunogenic fragment thereof along with a pharmaceutically acceptable carrier.

62. The vaccinating composition according to claim 61, wherein said infection is a sexually transmitted disease.

63. An antibody against Chlamydia trachomatis wherein said antibody is directed against the polypeptide according to claim 49 or an immunogenic fragment thereof.

64. A method for diagnosing a Chlamydia trachomatis infection in an animal wherein said method comprises: (a) providing an animal sample of a tissue suspected to be infected by Chlamydia trachomatis; (b) bringing said sample into contact with an antibody according to claim 63; and (c) detecting antigen-antibody complexion; wherein said complexion is indicative of a Chlamydia trachomatis infection in said animal.

65. A method of preventing or treating a Chlamydia infection in an animal, which comprises administering an effective amount of an antibody according to claim 63, or an immunogenic fragment thereof, to an animal in need thereof.

66. A method for diagnosing a Chlamydia trachomatis infection in an animal wherein said method comprises (a) providing a polypeptide according to claim 49, or an immunogenic fragment thereof, optionally labeled; (b) bringing said polypeptide or immunogenic fragment thereof into contact with a serum sample of said animal; and (c) detecting complexes formed between said polypeptide or immunogenic fragment thereof and antibodies contained in the serum sample; wherein said complexes are indicative of a Chlamydia trachomatis infection in said animal.

67. A method of detecting Chlamydia in an animal wherein said method comprises (a) providing an animal sample of a tissue suspected to be infected by Chlamydia; (b) adding a primer pair for a polypeptide according to claim 49 to the tissue sample; (c) amplifying a polynucleotide that encodes for the polypeptide which corresponds to the primer pair selected; and (d) detecting the presence of Chlamydia by the presence or absence of said polynucleotide.

68. A method of screening for an active molecule inhibiting the secretion of a secreted Chlamydia polypeptide, comprising (a) supplying an active molecule to a culture of Chlamydia; (b) growing or incubating said culture for a time and under conditions suitable for said active molecule to exert an activity upon said culture; (c) adding a primer pair for a polypeptide according to claim 49 to the culture; (d) amplifying a polynucleotide that encodes for the polypeptide which corresponds to the primer pair selected; and (e) detecting the presence of the secreted Chlamydia polypeptide by the presence or absence of said polynucleotide.

69. A plasmid for expression of secreted Chlamydia trachomatis polypeptide wherein said plasmid contains at least a polynucleotide coding for a polypeptide according to claim 49.

70. The plasmid according to claim 69, wherein said polynucleotide is further fused to a reporter gene.

71. The plasmid according to claim 70, wherein a vector deposited at C.N.C.M. on Dec. 13, 2000 with accession No. I-2593 is used for the construction of said plasmid.

72. A recombinant Gram-negative strain, wherein said strain is transformed by a plasmid according to claim 69.

73. The recombinant Gram-negative strain according to claim 72, wherein said strain is a Shigella strain.

74. A method of preventing or treating a Chlamydia infection in an animal, which comprises administering an effective amount of a purified secreted Chlamydia polypeptide, wherein said Chlamydia polypeptide is homologous to one or more Chlamydia trachomatis proteins selected from the group consisting of CT387, CT476, CT550, CT606.1, CT610, CT642, CT652.1, CT664, CT718, CT763, CT845, and CT848; or a fragment thereof.

75. The method according to claim 74, wherein said Chlamydia polypeptide is one or more Chlamydia trachomatis proteins selected from the group consisting of CT387, CT476, CT550, CT606.1, CT610, CT642, CT652.1, CT664, CT718, CT763, CT845, and CT848; or a fragment thereof.

76. The method according to claim 74, wherein said Chlamydia infection is a Chlamydia trachomatis infection.

77. The method according to claim 74, wherein said Chlamydia polypeptide is identified by its secretion in a Gram-negative bacterial strain containing a type III secretion pathway.

78. The method according to claim 74, wherein said animal is selected from the group consisting of a human, an equine, a bovine, a porcine, a caprine, a ovine, a bird, a dog, and a cat.

79. The method according to claim 74, wherein said animal is a human.

80. A purified secreted Chlamydia polypeptide, wherein said Chlamydia polypeptide is homologous to one or more Chlamydia psittaci proteins selected from the group consisting of Psi0330, Psi0379, Psi0595, Psi0648, Psi0671, Psi0705, Psi0710, Psi0761, Psi0774, Psi1002, Psi1005, Psi1022, and Psi1058; or a fragment thereof.

81. The polypeptide according to claim 80, wherein the homologous Chlamydia polypeptide is a Chlamydia psittaci protein.

82. The polypeptide according to claim 80, wherein said polypeptide is identified by its expression by a Gram-negative bacterial strain and secretion by the type III secretion pathway of said bacterial strain.

83. The polypeptide according to claim 80, wherein said Chlamydia polypeptide is one or more Chlamydia psittaci proteins selected from the group consisting of Psi0330, Psi0379, Psi0595, Psi0648, Psi0671, Psi0705, Psi0710, Psi0761, Psi0774, Psi1002, Psi1005, Psi1022, and Psi1058; or a fragment thereof.

84. The polypeptide according to claim 80, wherein said Gram-negative strain containing a type III secretion pathway is a Shigella strain.

85. The polypeptide according to claim 80, wherein said polypeptide is selected by a method for identifying polypeptides secreted by Chlamydia psittaci comprising (a) providing a recombinant expression vector containing at least the polynucleotide coding for the polypeptide of interest; (b) transforming a Gram-negative strain containing a type III secretion pathway with said recombinant vector; (c) expressing this vector in the Gram-negative strain transformed in (b); and (d) detecting the secretion of said polynucleotide expression product; wherein the secretion of said expression product indicates that it corresponds to a secreted Chlamydia psittaci polypeptide.

86. The polypeptide according to claim 85, wherein said Gram-negative strain containing a type III secretion pathway is a Shigella strain.

87. The polypeptide according to claim 80, wherein said polypeptide is selected by a method for identifying polypeptides secreted by Chlamydia psittaci comprising (a) providing a recombinant expression vector comprising at least the DNA coding for the polypeptide of interest fused to a reporter gene; (b) transforming a Gram-negative strain containing a type III secretion pathway with said recombinant vector; (c) expressing this vector in the Gram-negative strain transformed in (b); and (d) detecting the secretion of said reporter gene expression product; wherein the secretion of said expression product indicates that the fused DNA contains at least a polynucleotide corresponding to a secreted Chlamydia psittaci polypeptide.

88. The polypeptide according to claim 87, wherein said Gram-negative strain containing a type III secretion pathway is a Shigella strain.

89. A purified polynucleotide encoding at least one polypeptide according to claim 80 or a purified polynucleotide which hybridizes to said purified polynucleotide encoding at least one polypeptide according to claim 80 under stringent conditions.

90. The purified polynucleotide according to claim 89, wherein said polynucleotide is a primer or a probe.

91. An immunogenic composition comprising at least a polypeptide according to claim 80 or an immunogenic fragment thereof.

92. A vaccinating composition against Chlamydia psittaci infection wherein said composition comprises at least one polypeptide according to claim 80 or an immunogenic fragment thereof along with a pharmaceutically acceptable carrier.

93. The vaccinating composition according to claim 92, wherein said infection is a sexually transmitted disease.

94. An antibody against Chlamydia psittaci, wherein said antibody is directed against the polypeptide according to claim 80 or an immunogenic fragment thereof.

95. A method for diagnosing a Chlamydia psittaci infection in an animal wherein said method comprises: (a) providing an animal sample of a tissue suspected to be infected by Chlamydia psittaci; (b) bringing said sample into contact with an antibody according to claim 94; and (c) detecting antigen-antibody complexion; wherein said complexion is indicative of a Chlamydia psittaci infection in said animal.

96. A method of preventing or treating a Chlamydia infection in an animal, which comprises administering an effective amount of an antibody according to claim 94, or an immunogenic fragment thereof, to an animal in need thereof.

97. A method for diagnosing a Chlamydia psittaci infection in an animal wherein said method comprises (a) providing a polypeptide according to claim 80, or an immunogenic fragment thereof, optionally labeled; (b) bringing said polypeptide or immunogenic fragment thereof into contact with a serum sample of said animal; and (c) detecting complexes formed between said polypeptide or immunogenic fragment thereof and antibodies contained in the serum sample; wherein said complexes are indicative of a Chlamydia psittaci infection in said animal.

98. A method of detecting Chlamydia in an animal wherein said method comprises (a) providing an animal sample of a tissue suspected to be infected by Chlamydia; (b) adding a primer pair for a polypeptide according to claim 80 to the tissue sample; (c) amplifying a polynucleotide that encodes for the polypeptide which corresponds to the primer pair selected; and (d) detecting the presence of Chlamydia by the presence or absence of said polynucleotide.

99. A method of screening for an active molecule inhibiting the secretion of a secreted Chlamydia polypeptide, comprising (a) supplying an active molecule to a culture of Chlamydia; (b) growing or incubating said culture for a time and under conditions suitable for said active molecule to exert an activity upon said culture; (c) adding a primer pair for a polypeptide according to claim 80 to the culture; (d) amplifying a polynucleotide that encodes for the polypeptide which corresponds to the primer pair selected; and (e) detecting the presence of the secreted Chlamydia polypeptide by the presence or absence of said polynucleotide.

100. A plasmid for expression of secreted Chlamydia psittaci polypeptide, wherein said plasmid contains at least DNA coding for a polypeptide according to claim 80.

101. The plasmid according to claim 100, wherein said DNA is further fused to a reporter gene.

102. The plasmid according to claim 101, wherein a vector deposited at C.N.C.M. on Dec. 13, 2000 with accession No. I-2593 is used for the construction of said plasmid.

103. A recombinant Gram-negative strain, wherein said strain is transformed by a plasmid according to claim 100.

104. The recombinant Gram-negative strain according to claim 103, wherein said strain is a Shigella strain.

105. A method of preventing or treating a Chlamydia infection in an animal, which comprises administering an effective amount of a purified secreted Chlamydia polypeptide, wherein said Chlamydia polypeptide is homologous to one or more Chlamydia psittaci proteins selected from the group consisting of Psi0330, Psi0379, Psi0595, Psi0648, Psi0671, Psi0705, Psi0710, Psi0761, Psi0774, Psi1002, Psi 1005, Psi1022, and Psi1058; or a fragment thereof.

106. The method according to claim 105, wherein said Chlamydia polypeptide is one or more Chlamydia psittaci proteins selected from the group consisting of Psi0330, Psi0379, Psi0595, Psi0648, Psi0671, Psi0705, Psi0710, Psi0761, Psi0774, Psi1002, Psi1005, Psi1022, and Psi1058; or a fragment thereof.

107. The method according to claim 105, wherein said Chlamydia infection is a Chlamydia psittaci infection.

108. The method according to claim 105, wherein said Chlamydia polypeptide is identified by its secretion in a Gram-negative bacterial strain containing a type III secretion pathway.

109. The method according to claim 105, wherein said animal is selected from the group consisting of a human, an equine, a bovine, a porcine, a caprine, a ovine, a bird, a dog, and a cat.

110. The method according to claim 105, wherein said animal is a human.

111. A purified polynucleotide obtainable by a method comprising (a) contacting a Chlamydia DNA fragment with a primer pair selected from the group consisting of:

| Forward primer | Reverse primer |
|---|---|
| SEQ ID NO: 133 | SEQ ID NO: 250 |
| SEQ ID NO: 134 | SEQ ID NO: 251 |
| SEQ ID NO: 135 | SEQ ID NO: 252 |
| SEQ ID NO: 136 | SEQ ID NO: 253 |
| SEQ ID NO: 137 | SEQ ID NO: 254 |
| SEQ ID NO: 138 | SEQ ID NO: 255 |
| SEQ ID NO: 139 | SEQ ID NO: 256 |
| SEQ ID NO: 140 | SEQ ID NO: 257 |
| SEQ ID NO: 141 | SEQ ID NO: 258 |
| SEQ ID NO: 142 | SEQ ID NO: 259 |
| SEQ ID NO: 143 | SEQ ID NO: 260 |
| SEQ ID NO: 144 | SEQ ID NO: 261 |
| SEQ ID NO: 145 | SEQ ID NO: 262 |
| SEQ ID NO: 146 | SEQ ID NO: 263 |
| SEQ ID NO: 147 | SEQ ID NO: 264 |
| SEQ ID NO: 148 | SEQ ID NO: 265 |
| SEQ ID NO: 149 | SEQ ID NO: 266 |
| SEQ ID NO: 150 | SEQ ID NO: 267 |
| SEQ ID NO: 151 | SEQ ID NO: 268 |
| SEQ ID NO: 152 | SEQ ID NO: 269 |
| SEQ ID NO: 153 | SEQ ID NO: 270 |
| SEQ ID NO: 154 | SEQ ID NO: 271 |
| SEQ ID NO: 155 | SEQ ID NO: 272 |
| SEQ ID NO: 156 | SEQ ID NO: 273 |
| SEQ ID NO: 157 | SEQ ID NO: 274 |
| SEQ ID NO: 158 | SEQ ID NO: 275 |
| SEQ ID NO: 159 | SEQ ID NO: 276 |
| SEQ ID NO: 160 | SEQ ID NO: 277 |
| SEQ ID NO: 161 | SEQ ID NO: 278 |
| SEQ ID NO: 162 | SEQ ID NO: 279 |
| SEQ ID NO: 163 | SEQ ID NO: 280 |
| SEQ ID NO: 164 | SEQ ID NO: 281 |
| SEQ ID NO: 165 | SEQ ID NO: 282 |
| SEQ ID NO: 166 | SEQ ID NO: 283 |
| SEQ ID NO: 167 | SEQ ID NO: 284 |
| SEQ ID NO: 168 | SEQ ID NO: 285 |
| SEQ ID NO: 169 | SEQ ID NO: 286 |
| SEQ ID NO: 170 | SEQ ID NO: 287 |
| SEQ ID NO: 171 | SEQ ID NO: 288 |
| SEQ ID NO: 172 | SEQ ID NO: 289 |
| SEQ ID NO: 173 | SEQ ID NO: 290 |
| SEQ ID NO: 174 | SEQ ID NO: 291 |
| SEQ ID NO: 175 | SEQ ID NO: 292 |
| SEQ ID NO: 176 | SEQ ID NO: 293 |
| SEQ ID NO: 177 | SEQ ID NO: 294 |
| SEQ ID NO: 178 | SEQ ID NO: 295 |
| SEQ ID NO: 179 | SEQ ID NO: 296 |
| SEQ ID NO: 180 | SEQ ID NO: 297 |
| SEQ ID NO: 181 | SEQ ID NO: 298 |
| SEQ ID NO: 182 | SEQ ID NO: 299 |
| SEQ ID NO: 183 | SEQ ID NO: 300 |
| SEQ ID NO: 184 | SEQ ID NO: 301 |
| SEQ ID NO: 185 | SEQ ID NO: 302 |
| SEQ ID NO: 186 | SEQ ID NO: 303 |
| SEQ ID NO: 187 | SEQ ID NO: 304 |
| SEQ ID NO: 188 | SEQ ID NO: 305 |
| SEQ ID NO: 189 | SEQ ID NO: 306 |
| SEQ ID NO: 190 | SEQ ID NO: 307 |
| SEQ ID NO: 191 | SEQ ID NO: 308 |
| SEQ ID NO: 192 | SEQ ID NO: 309 |
| SEQ ID NO: 193 | SEQ ID NO: 310 |
| SEQ ID NO: 194 | SEQ ID NO: 311 |
| SEQ ID NO: 195 | SEQ ID NO: 312 |
| SEQ ID NO: 196 | SEQ ID NO: 313 |
| SEQ ID NO: 197 | SEQ ID NO: 314 |
| SEQ ID NO: 198 | SEQ ID NO: 315 |
| SEQ ID NO: 199 | SEQ ID NO: 316 |
| SEQ ID NO: 200 | SEQ ID NO: 317 |
| SEQ ID NO: 201 | SEQ ID NO: 318 |
| SEQ ID NO: 202 | SEQ ID NO: 319 |
| SEQ ID NO: 203 | SEQ ID NO: 320 |
| SEQ ID NO: 204 | SEQ ID NO: 321 |
| SEQ ID NO: 205 | SEQ ID NO: 322 |
| SEQ ID NO: 206 | SEQ ID NO: 323 |
| SEQ ID NO: 207 | SEQ ID NO: 324 |
| SEQ ID NO: 208 | SEQ ID NO: 325 |
| SEQ ID NO: 209 | SEQ ID NO: 326 |
| SEQ ID NO: 210 | SEQ ID NO: 327 |
| SEQ ID NO: 211 | SEQ ID NO: 328 |
| SEQ ID NO: 212 | SEQ ID NO: 329 |
| SEQ ID NO: 213 | SEQ ID NO: 330 |
| SEQ ID NO: 214 | SEQ ID NO: 331 |
| SEQ ID NO: 215 | SEQ ID NO: 332 |
| SEQ ID NO: 216 | SEQ ID NO: 333 |
| SEQ ID NO: 217 | SEQ ID NO: 334 |
| SEQ ID NO: 218 | SEQ ID NO: 335 |
| SEQ ID NO: 219 | SEQ ID NO: 336 |
| SEQ ID NO: 220 | SEQ ID NO: 337 |
| SEQ ID NO: 221 | SEQ ID NO: 338 |
| SEQ ID NO: 222 | SEQ ID NO: 339 |
| SEQ ID NO: 223 | SEQ ID NO: 340 |
| SEQ ID NO: 224 | SEQ ID NO: 341 |
| SEQ ID NO: 225 | SEQ ID NO: 342 |
| SEQ ID NO: 226 | SEQ ID NO: 343 |
| SEQ ID NO: 227 | SEQ ID NO: 344 |
| SEQ ID NO: 228 | SEQ ID NO: 345 |
| SEQ ID NO: 229 | SEQ ID NO: 346 |
| SEQ ID NO: 230 | SEQ ID NO: 347 |
| SEQ ID NO: 231 | SEQ ID NO: 348 |
| SEQ ID NO: 232 | SEQ ID NO: 349 |
| SEQ ID NO: 233 | SEQ ID NO: 350 |
| SEQ ID NO: 234 | SEQ ID NO: 351 |
| SEQ ID NO: 235 | SEQ ID NO: 352 |
| SEQ ID NO: 236 | SEQ ID NO: 353 |
| SEQ ID NO: 237 | SEQ ID NO: 354 |
| SEQ ID NO: 238 | SEQ ID NO: 355 |
| SEQ ID NO: 239 | SEQ ID NO: 356 |
| SEQ ID NO: 240 | SEQ ID NO: 357 |
| SEQ ID NO: 241 | SEQ ID NO: 358 |
| SEQ ID NO: 242 | SEQ ID NO: 359 |
| SEQ ID NO: 243 | SEQ ID NO: 360 |
| SEQ ID NO: 244 | SEQ ID NO: 361 |
| SEQ ID NO: 245 | SEQ ID NO: 362 |
| SEQ ID NO: 246 | SEQ ID NO: 363 |
| SEQ ID NO: 247 | SEQ ID NO: 364 |
| SEQ ID NO: 248 | SEQ ID NO: 365 |
| SEQ ID NO: 249 | SEQ ID NO: 366 |
| SEQ ID NO: 367 | SEQ ID NO: 373 |
| SEQ ID NO: 368 | SEQ ID NO: 374 |
| SEQ ID NO: 369 | SEQ ID NO: 375 |
| SEQ ID NO: 370 | SEQ ID NO: 376 |
| SEQ ID NO: 371 | SEQ ID NO: 377 |
| SEQ ID NO: 372 | SEQ ID NO: 378 |

and (b) amplifying a polynucleotide that encodes for the polypeptide which corresponds to the primer pair selected.

112. A method for detecting Chlamydia in an animal comprising (a) providing an animal sample of a tissue suspected to be infected by Chlamydia; (b) adding a hybridization probe that hybridizes with a polynucleotide that encodes a Chlamydia polypeptide under stringent conditions; and (c) detecting the presence of Chlamydia by the presence or absence of said polynucleotide,
wherein said Chlamydia polypeptide is homologous to a protein selected from the group consisting of Chlamydia pneumoniae proteins selected from the group consisting of CPn0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn0939, CPn1002, CPn1005, CPn1007, CPn1019, CPn1020, CPn1032, and CPn1058; Chlamydia trachomatis proteins selected from the group consisting of CT387, CT476, CT550, CT606.1, CT610, CT642, CT652.1, CT664, CT718, CT763, CT845, and CT848; and Chlamydia psittaci proteins selected from the group consisting of Psi0330, Psi0379, Psi0595, Psi0648, Psi0671, Psi0705, Psi0701, Psi0761, Psi0774, Psi1002, Psi1005, Psi1022, and Psi1058; or a fragment thereof.

113. A method of detecting Chlamydia in an animal comprising (a) providing an animal sample of a tissue suspected to be infected by Chlamydia; (b) adding an antibody; optionally labeled, that forms a complex with a Chlamydia polypeptide under conditions suitable for complex formation; and (c) detecting the presence of Chlamydia by the presence or absence of said polypeptide,
wherein said Chlamydia polypeptide is homologous to a protein selected from the group consisting of Chlamydia pneumoniae proteins selected from the group consisting of CPn0104, CPn0206, CPn0210, CPn0399, CPn0405, CPn0443, CPn0480, CPn0489, CPn0490, CPn0497, CPn0522, CPn0556, CPn0582, CPn0588, CPn0595, CPn0671, CPn0673, CPn0681, CPn0712, CPn0720, CPn0725, CPn0729, CPn0746, CPn0755, CPn0761, CPn0764, CPn0770, CPn0774, CPn0792, CPn0853, CPn0859, CPn0879, CPn0906, CPn0939, CPn1002, CPn1005, CPn1007, CPn1019, CPn1020, CPn1032, and CPn1058; Chlamydia trachomatis proteins selected from the group consisting of CT387, CT476, CT550, CT606.1, CT610, CT642, CT652.1, CT664, CT718, CT763, CT845, and CT848; and Chlamydia psittaci proteins selected from the group consisting of Psi0330, Psi0379, Psi0595, Psi0648, Psi0671, Psi0705, Psi0710, Psi0761, Psi0774, Psi1002, Psi1005, Psi1022, and Psi1058; or a fragment thereof.
